(19) United States
(12) Patent Application Publication
(10) Pub. No.: US 2023/0253119 A1
(43) Pub. Date: Aug. 10, 2023

(54) COMPUTER-IMPLEMENTED METHOD

(71) Applicant: ROCKLEY PHOTONICS LIMITED, Altrincham (GB)

(72) Inventors: Jennifer Lynn CORSO, Peoria, AZ (US); Philip Perea, Aliso Viejo, CA (US)

(21) Appl. No.: 18/055,377

(22) Filed: Nov. 14, 2022

Related U.S. Application Data
(60) Provisional application No. 63/279,633, filed on Nov. 15, 2021.

(51) Int. Cl. G16H 50/30; A61B 5/00; A61B 5/01; G16H 40/67

(52) U.S. Cl. CPC G16H 50/30; A61B 5/6801; A61B 5/01; A61B 5/4331; A61B 5/4875; A61B 5/4866; G16H 40/67

(57) ABSTRACT

A computer-implemented method for deriving a physiological rank indicative of a physiological status of a user, the computer-implemented method comprising acquiring, from a sensor on a wearable device worn by a user, data including bodily parameter data related to the user, and applying a model to the bodily parameter data to obtain physiological information related to the user, and deriving, from the physiological information, a physiological rank indicative of a physiological status of the user wearing the device, wherein the physiological rank is a given value on a physiological rank scale.

### CROSS-REFERENCE TO RELATED APPLICATION(S)

The present application claims priority to and the benefit of U.S. Provisional Application No. 63/279,633, filed Nov. 15, 2021, entitled “COMPUTER-IMPLEMENTED METHOD FOR A WEARABLE DEVICE”, the entire content of which is incorporated herein by reference.

### FIELD

One or more aspects of embodiments according to the present invention relate to a computer-implemented method for deriving a physiological rank indicative of a physiological status of a user

### BACKGROUND

Physiological regulation of core body temperature is driven by the balance of energy exchange between the body and its surroundings. Mammals are homeotherms, and a balance between the loss and gain of heat results in what is defined as a homeostatic core temperature. In humans, core temperature must be maintained within a narrow range in order to prevent physiological breakdown, risking thermal injury, illness, or even death.

In healthy adults, a normal, basal core temperature range is considered to be between 36.2-37.7° C. Age, sex, body composition, activity, respiration rate, feeding status, environmental exposure, menstruation, and metabolic rate all affect a person's core temperature. Common, current methods of measurement attempt to estimate core body temperature but are often invasive or have significant deviations in measurement accuracy due to location of measure and the impact of the external environment.

Hydration status may have a significant impact in several areas, including a person's mood, physical and mental performance, kidney function, and skin condition. Understanding body hydration and when it might not be balanced properly can be extremely valuable for managing personal health and well-being.

However, the model surrounding hydration is exceptionally complex. Fluid balance is sustained through the coordination of many substances and mechanisms working in concert, with a heavy reliance upon neuroendocrine responses and healthy renal function. Modifying any one part of the fluid environment through dehydration or overhydration may elicit profound effects on hemodynamics and overall function.

As it stands, there exists no “gold standard measurement” for assessing hydration status, and therefore, no simplified index. To determine a person's level of hydration, various costly and time-consuming laboratory tests are employed to assess the osmolarity and electrolyte concentrations in physiological fluids such as urine and blood. Laboratory tests may include urine osmolality (UO), urine specific gravity (USG), serum osmolality (SO), fluid gain or weight gain and fluid loss or weight loss.

Fluid imbalances may lead to serious physical complications and present in a variety of common scenarios where laboratory testing is not available, timely, nor practical.

Current market options for monitoring analyte concentrations in a person's body such as blood glucose levels require a prescription or are only approved for use in clinical populations and in point of care settings. Thus, healthy people looking to incorporate analyte concentration monitoring into their health regimen without clinical oversight have few options. For example, healthy and prediabetic consumers looking to incorporate glucose monitoring into their health regimen without clinical oversight have few options.

### SUMMARY

Accordingly, embodiments of the present invention provide a computer-implemented method for deriving a physiological rank indicative of a physiological status of a user, the computer-implemented method comprising acquiring, from a sensor on a wearable device worn by a user, data including bodily parameter data related to the user, and applying a model to the bodily parameter data to obtain physiological information related to the user, and deriving, from the physiological information, a physiological rank indicative of a physiological status of the user wearing the device, wherein the physiological rank is a given value on a physiological rank scale.

Thus, an indication of the physiological status of a user can be derived from wearable device-acquired data. In this way, an indication of the physiological status of the user may be provided during a user's normal routine. Advantageously then, an indication of the physiological status of the user may be provided in a more accessible way than can be provided by laboratory tests.

Optional features of the computer-implemented method will now be described. The computer-implemented method may have anyone, or any combination insofar as they are compatible, of the following features.

The physiological rank may include a physiological index. The physiological index may be a given value on a physiological index scale. A value may mean a numerical value.

In this way, a quantitative assessment of physiological status may be provided.

The physiological rank may include a physiological status of the user, wherein the physiological status is a given grade on a physiological status scale.

In this way, a qualitative assessment of physiological status may be provided.

The physiological rank may include both the physiological index and the physiological status of the user. The model may include an index model which derives the physiological index, and a status model which derives the physiological status. The model may further include a confidence model which calculates a confidence value for the derived physiological index.

The physiological rank may be an oxygen saturation (SpO2) rank, a heart rate rank, a heart rate variability rank, a respiratory rate rank, a blood pressure rank, a core body temperature rank, a hydration rank, a health score, an analyte concentration value (wherein the analyte may be a metabolic fuel such as glucose, lactate, or ethanol), and a movement rank.

The physiological information may be a measure of a physiological indicator. The physiological indicator may be tissue perfusion or ischemia, infection, decompensation, pain, performance, overtraining, movement/activity, core body temperature, resting heart rate, real-time heart rate, maximum heart rate, heart rate thresholds, VO₂, VO₂ maximum or peak, intensity, sleep quality, sleep disturbance, apnea hypopnea index (AHI), oxygen desaturation index (ODI), metabolic equivalent of tasks (METs), metabolic health, caloric cost, or general health status.

Therefore, embodiments of the present invention may have applications in chronic disease risk reduction, lifestyle management, including the management of diet and physical activity, heat illness, fluid intake, cognitive performance, physical performance and training, stress, fatigue and recovery, trauma and shock, clinical research, and disease and illness monitoring.

As such, the present invention may be used by in-patient practitioners, surgeons, field medics, emergency medical technicians, research institutions, military departments of defense, public safety officers, transport professionals, sports trainers, athletes, agricultural workers, builders and construction workers, mining workers, oil workers, mill workers, welding workers, technical professionals and teachers.

The physiological information may be temperature information and the physiological rank may be a temperature rank of the user. The temperature rank may be an output of a temperature value or a temperature status. A temperature value may be a quantitative value such as a temperature in degrees Celsius or in degrees Fahrenheit. A temperature value may be referred to as a temperature. A temperature status may be a qualitative indication of the temperature of the user such as hypothermia, basal temperature, hyperthermia, fever, and hyperpyrexia.

In this way, a computer-implemented method may be provided which provides an indication of the temperature of the user.

Use of the methods and devices described herein may therefore contribute to: decreased risk of accidental hyperthermia; decreased risk of dehydration; decreased risk of hypothermia; improved awareness of potential illness; decreased illness spread with heightened awareness in high-risk populations; assisting public health infection trends and epidemiology; avoiding or improving chances or conception; assisting detection of fertility problems; decreased fetal risk during thermal stressors; predicting hot flushes; improving in-patient quality of life; faster notifications of changes through real time, continuous monitoring of vitals; improved awareness of post-surgical infection, cerebral injury and health care associated infections.

Further, the computer-implemented method may assist a user in understanding the impact of their behavior on their health status. For example, if a pregnant user decides to conduct physical activity outside on a warm day, they may assess their core body temperature using the computer-implemented method both during and after exercise to understand whether the exercise is causing their core body temperature to be in a safe zone in which the fetus is not at risk or in an unsafe zone in which the fetus is at risk.

A use case for the computer-implemented invention may be selectable by the user. Selectable use cases may include thermoregulation, infection/fever determination, women's health and critical care.

The thermoregulation use case may benefit users who are athletes; users with an occupational risk of dehydration/thermal injury such as users in the military, the police, and construction workers; vulnerable populations such as the elderly, users with cognitive impairment, or users with autoimmune disorders; users undergoing acclimation in a hot/cold/tropic environment. The infection and fever use case may benefit vulnerable users such as users who are immunocompromised, users who are pregnant, patients with cancer, and users who are travelling; users with high occupational risk such as teachers, healthcare workers, and users who work in commercial/mass transportation. The infection and fever use case may further benefit public health by e.g., tracking infection trends. The women's health use case may benefit users wishing to monitor their fertility or their menstrual cycle. The women's health use case may assist in monitoring/determining ovulation, pregnancy or menopause. The critical care use case may benefit users who are in hospital, such as users suffering a cerebral injury, users in intensive care, users in an emergency room, users in peri-operative monitoring, users undergoing dialysis, users who have suffered heart failure, users with severe infection or virus, users who are high risk of healthcare associated infections e.g., users who are post-transplant.

The required range of temperatures which can be derived from data acquired by the sensor may vary depending on the use case. For example, the required range of temperatures for the use case of women's health may be 35.0° C. to 41.1° C.; the required range of temperatures for monitoring/determining ovulation may be 36.0° C. to 38.0° C., the required range of temperatures for monitoring/determining pregnancy may be 35.0° C. to 41.1° C., and the required range of temperatures for monitoring/determining menopause may be 36.0° C. to 39.0° C. The required range of temperatures for the use case of infection/fever determination may be 36.0° C. to 40.0° C. The required range of temperatures for the use case of thermoregulation may be 35.0° C. to 40.0° C. The required range of temperatures of the use case of critical care may be 28.0° C. to 43.0° C.

The computer-implemented method may be carried out on the wearable device, or on an external device. The external device may be a mobile device such as a mobile phone. The wearable device may be any device worn on the body, for example a watch, a ring, a necklace, bracelet, an ear bud, a skin contact patch, a glasses frame, or a strap worn around the wrist, the arm, the leg, or the torso.

The bodily parameter data may be an optical spectrum. The bodily parameter data may be an absorption spectrum. The bodily parameter data may be a body tissue absorption spectrum. The absorption may be in the water band. The bodily parameter data may be a reflectance spectrum. The bodily parameter data may be a body tissue reflectance spectrum. The bodily parameter data may be an infrared diffuse reflectance spectrum.

The physiological information may be a quantitative value associated with the bodily parameter data. The physiological information may be a quantitative value associated with the optical spectrum. For example, the physiological information may be a position of a peak of the optical spectrum, for example a wavelength shift of a peak of the optical spectrum, a height of a peak of the optical spectrum, or a width of a peak of the optical spectrum.

Each physiological rank on the physiological rank scale may map onto a respective output of a standard clinical test. The output of a standard clinical test may be a clinical measurement indicative of the physiological status of a user. For example, when the physiological rank is a temperature rank, the standard clinical test may use an oral thermometer to make a temperature measurement.

In this way, a physiological rank may be derived which provides a clinically relevant indication of physiological status.

The computer-implemented method may continuously acquire data. The computer-implemented method may continuously derive the physiological rank. The computer-implemented method may acquire data at pre-determined time points, for example the data may be acquired every hour, every 6 hours, every 12 hours or every 24 hours. The computer-implemented method may acquire data upon receiving a user input signal. The computer-implemented method may derive the physiological rank at predetermined time points, for example the hydration rank may be derived every hour, every 6 hours, every 12 hours or every 24 hours. The computer-implemented method may derive the physiological rank upon receiving a user input signal.

Whether data is acquired continuously or intermittently may depend on the use case. For example, in the use case of infection/fever determination, data may be acquired intermittently. In the use case of critical care, data may be acquired continuously.

The sensor may be an optical sensing module (i.e. an optical sensor) on the wearable device.

In this way, an indication of clinical physiological status may be derived using an optical measurement. Further, an indication of the physiological status of the user may be provided non-invasively. An indication of the physiological status of the user may be provided without requiring a sample to be taken from the user. Further, when the physiological rank is a temperature rank, for example, an indication of the core temperature of the user may be provided which is more accurate than existing methods for measuring core body temperature, while being non-invasive.

The optical sensing module may comprise a laser. The optical sensing module may comprise a plurality of lasers. Each laser of the plurality of lasers may operate at a wavelength that is different from the wavelength of the others. The optical sensing module may be configured to operate each laser one at a time. The optical sensing module may be configured to operate the plurality of lasers in a cycle according to a pre-determined schedule.

In this way, the optical sensing module may require less detectors. The optical sensing module may require only one detector. Advantageously then, the optical sensing module may be cheaper and simpler to manufacture than an optical sensing module which requires more detectors.

The optical sensing module may have a sampling rate of 50,000 samples per second or 1,000 samples per second. The data acquired from the optical sensing module may be an average of the samples. A laser on-time for the laser or for each of the plurality of lasers be 200 microseconds. The number of samples acquired in 200 microseconds may be 10. Two samples may be discarded in the processing. A laser off-time for the laser or for each of the plurality of lasers may be 100 microseconds. The number of samples acquired in 100 microseconds may be 5. A time to perform 60 cycles may be 20 milliseconds. The number of samples acquired in 20 milliseconds may be 1000. A total measurement time may be 10 seconds. The number of cycles in 10 seconds may be 500. A plurality of total measurements may be taken. There may be an interval of 15 seconds between each total measurement.

The laser or the plurality of lasers may emit light in a wavelength band which is sensitive to changes in water concentration within the interstitial space. The laser or the plurality of lasers may emit light in the wavelength band which covers wavelengths between at least 350 nm and no more than 2500 nm. The laser or the plurality of lasers may emit light in the visible wavelength band. The visible wavelength band may cover wavelengths roughly from 300 nm to 780 nm. The laser or the plurality of lasers may emit light in the infrared wavelength band. The laser or the plurality of lasers may emit light in the near-infrared wavelength band. The near infrared wavelength band may cover wavelengths roughly from 780 nm to 1000 nm. The laser or the plurality of lasers may emit light in the short wavelength infrared wavelength band. The short wavelength infrared wavelength band may cover wavelengths from roughly from 1000 nm to 2500 nm. The laser or a laser within the plurality of lasers may emit light at a wavelength suitable for detecting data including bodily parameter data from which the physiological information can be derived. For example, if the physiological rank is a temperature rank the laser or a laser in the plurality of lasers may emit light in a short wavelength infrared wavelength band. The laser or a laser within the plurality of lasers may emit light at e.g. 970 nm, 1200 nm, 1450 nm, 1950 nm, 2766 nm, 2898 nm, or 6097 nm.

The optical sensing module may comprise one or more optical outputs for light originating from the laser or the plurality of lasers. Light from the laser or the plurality of lasers may exit the optical sensing module via one or more optical output ports. The optical sensing module may comprise a mirror to take the light from the plane of the optical sensing module and translate it into a direction more suitable for interrogating the surface. The direction may be orthogonal or substantially orthogonal to the plane of the optical sensing module.

The optical sensing module may include a transmitter photonic integrated circuit (PIC). The optical sensing module may comprise a substrate. The substrate may be a silicon substrate. The transmitter PIC may be located on the substrate. The transmitter PIC may include the laser or the plurality of lasers. The transmitter photonic integrated circuit (PIC) may be a silicon or silicon nitride photonic integrated circuit.

In use, light emitted from a laser of the optical sensing module may reflect or backscatter from a layer of the skin of the user. The optical sensing module may be configured to receive light backscattered from the skin of the user.

The optical sensing module may comprise a detector. The detector may be a photodetector. The detector may be located on the transmitter PIC such that the PIC is a transmitter/receiver PIC. The detector may be located separately from the transmitter PIC. The photodetector may be a silicon-based photodetector. The photodetector may be an InGaAs-based photodetector. The photodetector may be a germanium photodetector. The photodetector may be located on a receiver PIC that is vertically integrated and mounted on the same substrate as the transmitter PIC. The optical sensing module may comprise a plurality of detectors.

The optical sensing module may comprise an optical manipulation region. The optical manipulation region may comprise one or more of an optical modulator, an optical multiplexer, and additional optical manipulation elements.

The optical sensing module may be that disclosed in WO 2021/116766, the contents of which are incorporated herein by reference in its entirety.

In this way, the optical sensing module may have the ability to continuously take data. Therefore, the computer-implemented method may continuously receive data. Therefore, the physiological status of the user may be continuously monitored. Further, the physiological rank of the user may be provided in real time.

The optical sensing module may comprise LEDs instead of lasers. LEDs may cheaper and simpler to manufacture than lasers. Lasers may allow for a more accurate indication of the physiological status of the user.

The bodily parameter data may be a body tissue absorption spectrum. The absorption may be in the water band. In some examples, the wavelength of the laser or a laser in the plurality of lasers may correspond to the wavelength of a water absorption peak.

The model may include a machine learning model. The model may include a regression model. The model may include a classifier. The model may include a logistic regression model. The model may include a partial least squares (PLS) regression model. The model may include a principal component analysis (PCA) model. The PCA model may be applied before the regression model. The PCA model may be applied before the classifier.

The model may have been trained using training data. The training data may include a plurality of training datasets, each of the training datasets comprising bodily parameter data, and each of the training datasets acquired form the wearable device. The training data may include clinical labels. Each training dataset may be associated with a respective one of the clinical labels. Each of the clinical labels may include an output of a standard clinical test. The clinical data which is used to derive the output of the standard clinical test may have been acquired at the same time as or at a similar time to the acquisition of the corresponding training dataset, for example within the same 5 minute interval, 15 minute interval, or 1 hour interval. Each of the clinical labels may include a plurality of outputs of a respective plurality of standard clinical tests.

In one or more embodiments, the training data is acquired from a plurality of subjects. In one or more embodiments, the training data is acquired from a single subject. The single subject may be the user of the wearable device.

The training data may be acquired during a training period. The training period may, for example, have a duration of 1 day to 21 days. The training period may be between 7 days and 14 days.

The model trained or generated in this way is not limited to a machine learning model.

The model may include an offline model. The offline model may have been trained using batch training data.

For example, when the physiological rank is a temperature rank, the training data may be acquired from the user of the wearable device during a training period. The clinical labels may be temperature labels which are temperature measurements obtained using a standard temperature test. The standard temperature test may include the use of a standard temperature instrument e.g., an oral thermometer, a non-contact IR thermometer, or an electric probe, to measure the temperature of the user at a standard location e.g., oral, forehead or tympanic.

The model may comprise one or more pre-processing steps. The one or more pre-processing steps may comprise applying a statistical model to the data acquired from the sensor to validate the data acquired from the sensor.

In one or more embodiments, the computer-implemented method may comprise a step of detecting whether the bodily parameter data has been acquired from a human body.

Validating the data may comprise determining whether the data has been acquired from a human user. In addition, or alternatively, validating the data may comprise determining whether the data is anomalous data.

In this way, the effect of any anomalous data, or any incorrectly acquired data on the accuracy of the physiological status indication is reduced.

The statistical model may be generated using a plurality of training datasets, each dataset comprising bodily parameter data. The plurality of training datasets may have been acquired from a plurality of subjects. To validate the data acquired from the sensor, the statistical model may determine whether the data acquired from the sensor falls within a pre-determined number of standard deviations, for example within 2 standard deviations, of the plurality of training datasets. To validate the data acquired form the sensor, the statistical model may calculate a Mahalonobis distance metric between the data acquired form the sensor and the plurality of training datasets, and determine whether the Mahalonobis distance metric is within a given threshold.

The one or more pre-processing steps may comprise applying a baseline correction to the data. Applying the baseline correction to the data may comprise subtracting baseline data from the acquired data. The baseline data may be derived from data previously acquired from the user. The baseline data may be average data acquired from the user. The average data may have been derived from data acquired from the user over a long time period, for example, over 24 hours, 1 week, or 1 month.

In this way, the effect of noise in the data may be reduced. In this way, the indication of the user's physiological status may be more accurate.

The computer-implemented method may comprise acquiring other sensor information in addition to the physiological information. The computer-implemented method may comprise acquiring user input information.

The other sensor information may be acquired from the sensor on the wearable device. The other sensor information may be acquired from an additional sensor. The additional sensor may be external to the wearable device. The additional sensor may be on the wearable device. The other sensor information may be obtained from other bodily parameter data related to the user. The other bodily parameter data may be an optical spectrum.

The other sensor information may include clinically relevant information. The other sensor information may include one or more of body temperature information obtained from a temperature sensor, heart rate information obtained from a heart rate sensor, blood oxygen saturation information obtained from a blood oxygen saturation sensor, respiratory rate information obtained from a respiratory rate sensor, hydration information obtained from a hydration sensor, accelerometer and motion information obtained from an accelerometer or a motion sensor, heart rate variability information obtained from a heart rate sensor, alcohol concentration, sleep/wake information obtained from a sleep sensor, blood pressure information obtained from a blood pressure sensor, analyte concentration information (wherein the analyte may be a metabolic fuel such as glucose, lactate, or ethanol) obtained from an analyte concentration sensor, and climate information obtained from a climate sensor (e.g., a thermometer).

The heart rate sensor, the blood oxygen saturation sensor and the respiration rate sensor may be a PPG sensor. The blood pressure sensor and the heart rate sensor may be an SPG sensor. The temperature sensor may be a short-wavelength infrared sensor. The heart rate information and the heart rate variability information may be obtained from an electrocardiogram.

The other sensor information may be or may be a measure of a physiological indicator. Physiological indicators may include tissue perfusion or ischemia, infection, decompensation, pain, performance, overtraining, movement/activity, core body temperature, resting heart rate, real-time heart rate, maximum heart rate, heart rate thresholds, VO₂ or VO₂ maximum, intensity, sleep quality, sleep disturbance, apnea hypopnea index (AHI), oxygen desaturation index (ODI), metabolic equivalent of tasks (METs), metabolic health, caloric cost, or general health status.

For example, the blood oxygen saturation information may be an oxygen desaturation index. The heart rate information may be a VO₂ measurement or a maximum VO₂ measurement. The heart rate information may be an MET measurement. The respiration rate information may be an apnea-hypopnea index or a respiratory disturbance index.

The other sensor information may be a secondary physiological rank. The secondary physiological rank may be a temperature rank, a hydration rank or a health score derived according to one or more embodiments of the present invention.

The user input information may be acquired from a user input into the wearable device. The user input information may be acquired from a user input into an external device which may be a mobile device such as a mobile phone. The user input information may include one or more of weight information, height information, activity information, diet information, fluid intake information, sodium intake information, illness information, intoxication information, blood pressure information, sleep duration information, sleep quality information, gender information, age information, heart rate information, cervical mucous information and date of menstruation information.

The user input information may include a value on a clinically relevant scale. For example, weight information may include a mass in kg, or pounds. Weight information may include a body mass index (BMI). Height information may include a height in cm or inches. Activity information may include an amount of calories burnt. Activity information may include information from which an amount of calories burnt could be calculated, for example a type of exercise and a duration of the exercise. Activity information may also include duration of an activity completed, type of activity completed or other information related to the user's experience of the activity, such as perceived exertion. Diet information may include a food group (such as fat, carbohydrate or protein) consumed. Diet information may include an amount of calories consumed. Diet information may include information from which an amount of calories consumed could be calculated, for example a type of food and an amount of food. Fluid intake information may include a volume of fluid consumed. Fluid intake information may include a type of fluid consumed, for example water or an electrolyte fluid. Illness information may include a temperature. Illness information may include a type of diagnosed illness, a duration of illness or other information related to symptoms. Intoxication information may include a number of alcohol units consumed. Intoxication information may include information from which a number of alcohol units consumed could be calculated, for example a type of alcohol and a volume consumed. Intoxication information may include a number of days in which alcohol has been consumed. Blood pressure information may include a measurement in mmHg. Blood pressure information may include a ratio of systolic pressure to diastolic pressure, where each pressure may be a measurement in mmHg.

The computer-implemented method may comprise acquiring a learnt basal physiological rank of the user or a learnt basal bodily parameter data of the user. Basal bodily parameter data may mean average bodily parameter data over a prolonged period for example over 24 hours, 1 week or 1 month. Basal bodily parameter data may be bodily parameter data acquired when the user is at rest. A basal physiological rank may mean an average physiological rank over a prolonged period for example over 24 hours, 1 week or 1 month. A basal physiological rank may be derived from data acquired when the user is at rest, the data including basal bodily parameter data.

The learnt basal physiological rank or basal bodily parameter data may be acquired from a memory. The memory may be located in the wearable device or in an external device which may be a mobile device such as a mobile phone.

The computer-implemented method may further comprise applying a basal physiological model to the bodily parameter data. The basal physiological model may take the learnt basal bodily parameter data as an input. The basal physiological model may derive whether the bodily parameter data is a pre-determined threshold away from the basal bodily parameter data of the user.

The computer-implemented method may further comprise applying a basal physiological model to the physiological rank. The basal physiological model may take the learnt physiological rank as an input. The basal physiological model may compare the derived physiological rank and the basal physiological rank and, based on this comparison, may determine whether the derived physiological rank is a pre-determined threshold away from a basal physiological rank of the user.

The computer-implemented method may comprise, when the basal physiological model determines that the bodily parameter data is more than the pre-determined threshold away from a user's basal bodily parameter data, alerting the user. The computer-implemented method may comprise, when the basal physiological model determines that the derived physiological rank is more than a pre-determined threshold away from a user's basal physiological rank, alerting the user.

For example, when the physiological rank is a temperature, the computer-implemented method may comprise, when the basal physiological model determines that the derived temperature is more than a pre-determined threshold away from a user's basal temperature, alerting the user. By notifying users of changes relative to their core temperature, enhancements may be made in the areas of physical performance, and risks may be mitigated related to infection severity, and heat or cold stress.

The alert may be output by the wearable device. The alert may be output by an external device. The alert may be a haptic, aural or a visual alert. For example, the alert may be a visual indication on the wearable device that the user is out of their basal physiological range.

In this way, a physical output may be provided to the user when the user is out of their basal physiological range.

The alert may be tailored to the use case. For example, in the women's health use case, if the derived temperature indicates that the user's temperature is above their basal temperature, the alert may indicate that the user is ovulating.

The learnt basal bodily parameter data or the learnt basal physiological rank may have been learnt using a machine learning model. The learnt basal bodily parameter data or the learnt basal physiological rank may have been learnt in a calibration period of the computer-implemented method. The calibration period may be an initial period in which a user is using the computer-implemented method. The calibration period may be between 1 day and 21 days. The calibration period may be between 7 days and 14 days. The calibration period may cover the entire period in which the user uses the computer-implemented method. The learnt basal bodily parameter data or the learnt basal physiological rank may have been learnt in a plurality of calibration sub-periods within the calibration period. Each calibration sub-period may be approximately 1 minute. Each calibration sub-period may be between 1 minute and 1 hour. Each calibration sub-period may be between 5 minutes and 15 minutes. Each of the calibration sub-periods may be a period in which the user has just woken up, for example a period in which the user has woken up within the last 5 minutes, 15 minutes, 30 minutes or 1 hour.

The learnt basal bodily parameter data of the user may have been learnt for changing other sensor information and/or for changing user input information. The learnt basal physiological rank of the user may have been learnt for changing other sensor information and/or for changing user input information. For example, when the physiological rank is temperature, different basal temperatures may be learnt for different points within the user's menstrual cycle may by taking into account other sensor information and/or user input information which is indicative of different points within the menstrual cycle such as dates of menstruation and cervical mucous information.

The training data used to learn the basal physiological rank or the basal bodily parameter data may include a plurality of training basal datasets, the training basal datasets including bodily parameter data, and the training basal datasets acquired from the wearable device. The training data may comprise a plurality of context labels. Each training basal dataset may be associated with a respective one of the context labels. Each of the context labels may include training other sensor information and/or training user input information. The training other sensor information may be acquired at the same time as or at a similar time to the acquisition of the corresponding training basal dataset, for example within the same 5 minute interval, 15 minute interval, or 1 hour interval. The training user input information be acquired at the same time as or at a similar time to the acquisition of the corresponding training basal dataset, for example within the same 5 minute interval, 15 minute interval, or 1 hour interval.

The training user input information may include weight information, height information, activity information, diet information, fluid intake information, sodium intake information, illness information, intoxication information, blood pressure information, sleep duration information, sleep quality information, gender information, age information, heart rate information, cervical mucous information and date of menstruation information. The training other sensor information may include body temperature information obtained from a temperature sensor, heart rate information obtained from a heart rate sensor, blood oxygen saturation information obtained from a blood oxygen saturation sensor, respiratory rate information obtained from a respiratory rate sensor, hydration information obtained from a hydration sensor, accelerometer and motion information obtained from an accelerometer or a motion sensor, heart rate variability information obtained from a heart rate sensor, alcohol concentration, sleep/wake information obtained from a sleep sensor, blood pressure information obtained from a blood pressure sensor, analyte concentration information (wherein the analyte may be a metabolic fuel such as glucose, lactate, or ethanol) obtained from an analyte concentration sensor, and climate information obtained from a climate sensor (e.g., a thermometer).

The parameters being learnt in this way does not limit the model used to learn the parameters to being a machine learning model.

In this way, basal bodily parameter data or a basal physiological rank of a user may be learnt. Further the basal bodily parameter data or the basal physiological rank may be correlated with user input information and/or other sensor information.

The computer-implemented method may comprise storing a notification data table.

The notification data table may associate recommendations with stored physiological ranks. The computer-implemented method may comprise comparing the derived physiological rank with the stored physiological ranks and, based on this comparison, selecting a notification to output to the user. The computer-implemented method may further comprise outputting the selected notification to the user.

Each stored physiological rank may be associated with one or more notifications. The selected notification may be a notification associated with the stored physiological rank which matches the derived physiological rank.

The physiological rank indexes may be sub-divided into a plurality of sub-ranges of physiological indexes.

The computer-implemented method may comprise determining a sub-range of physiological indexes which the derived physiological indexes falls within. The notification data table may associate recommendations with stored sub-ranges of physiological indexes. The computer-implemented method may comprise comparing the determined sub-range with the stored sub-ranges and, based on this comparison, selecting a notification to output to the user. The computer-implemented method may further comprise outputting the selected notification to the user.

Each stored sub-range may be associated with one or more notifications. The selected notification may be a notification associated with the stored sub-range which matches the determined sub-range.

The sub-ranges may have clinically relevant thresholds. For example, when the physiological rank is temperature, the sub-ranges may have thresholds relevant to basal temperatures, hyperthermic temperatures, hypothermic temperatures, fever temperatures, fever of unknown origin (FUO) temperatures, hyperpyrexia temperatures. The sub-ranges may have clinical thresholds relevant during pregnancy and during menstrual cycle phase changes.

The notifications may be recommended actions for the user to take, for example “drink water” or “stop consuming water”. The selected notification may be a recommendation which is clinically understood to improve the physiological status of the user. In this way, a notification appropriate to improving the clinical physiological status of the user may be output to the user.

The selected notification may be an indication of a clinical attribute of the user e.g., that the user is in an ovulation window or that the user is pregnant, ovulating, infected/ill, is hypothermic or is hyperthermic.

The sub-ranges may depend on the use case. The notifications associated with each sub-range may depend on the use-case.

For example, for the thermoregulation use case, the sub-ranges may include a sub-range of temperatures ≤35.0° C., which indicates hypothermia, a sub-range of temperatures >35.0° C. to <37° C., which indicates a mildly decreased temperature, a sub-range of temperatures ≥36.6° C. to ≤37.7° C., which indicates basal temperature, a sub-range of temperatures >37.7° C. to <38.0° C., which indicates a mildly elevated temperature, a sub-range of temperatures ≥38.0° C. to <40.0° C., which indicates a moderately elevated temperature, and a sub-range of temperatures ≥40.0° C., which indicates a severely elevated temperature.

For the thermoregulation use case, the notifications associated with the sub-range of temperatures ≤35.0° C. may include a notification to the user that their temperature has significantly decreased, a prompt for the user to seek assistance or medical attention, a prompt for the user to move around, to put on additional clothing, to drink warm fluids, or to move to a warmer location. The notifications associated with the sub-range of temperatures >35.0° C. to <37° C. may include a notification to the user that their temperature has decreased, a prompt for the user to add additional clothing or a blanket, a prompt for the user to drink warm fluids, a prompt for the user to move around, or a prompt for the user to move to a warmer location. The notifications associated with the sub-range of temperatures >37.7° C. to <38.0° C. may be a notification to the user that their temperature is slightly elevated, a prompt for the user to consume electrolyte fluid or water, a prompt for the user to continue to monitor their temperature and consume fluids. The notifications associated with the sub-range of temperatures ≥38.0° C. to <40.0° C. may include a notification to the user that their temperature is elevated, a prompt for the user to consume electrolyte fluid or water, a prompt for the user to decrease their activity, a prompt for the user to seek a cooler environment, or a prompt for the user to continue to monitor their temperature. The notifications associated with the sub-range of temperatures ≥40.0° C. may include a notification to the user that their temperature is significantly elevated, a prompt for the user to cease their activity, a prompt for the user to consume electrolyte fluid or water, or a prompt for the user to seek assistance or medical attention.

For the infection/fever determination use case, the sub-ranges may include a sub-range of temperatures ≥36.2° C. to <37.7° C., which indicates basal temperature, a sub-range of temperatures >37.7° C. to <38.0° C., which indicates a mildly elevated temperature, a sub-range of temperatures ≥38.0° C. to <40.0° C., which indicates febrile (fever) temperature, and a sub-range of ≥40.0° C. which indicates hyperpyrexia.

For the infection/fever determination use case, the notifications associated with the sub-range of temperatures >37.7° C. to <38.0° C. may include a notification to the user that their temperature is slightly elevated, a prompt for the user to input whether they are experiencing any other symptoms, a prompt for the user to continue to monitor their temperature, and a notification to the user that they may have an infection. The notifications associated with the sub-range of temperatures ≥38.0° C. to <40.0° C. may include a notification to the user that their temperature is elevated, a prompt for the user to input whether they are experiencing any other symptoms, a prompt for the user to consume water, and a prompt for the user to continue to monitor their temperature and to seek medical attention if their temperature remains elevated or increases. The notifications associated with the sub-range of temperatures ≥40.0° C. may include a notification to the user that their temperature is significantly elevated, a prompt for the user to consume water, or a prompt for the user to seek assistance or medical attention.

For the women's health use case, the sub-ranges may include a sub-range of follicular phase user-specific temperatures, which indicates basal temperature in the follicular phase of the menstrual cycle, where the sub-range is within the range ≥36.2° C. to ≤37.7° C., a sub-range of luteal phase user-specific temperatures, which indicates basal temperature in the luteal phase, where the sub-range of user-specific temperatures is within the range ≥36.2° C. to ≤37.7° C., a sub-range of temperatures >37.7° C. to <38.0° C., which indicates a mildly elevated temperature, a sub-range of temperatures ≥38.0° C., which indicates a febrile temperature or hyperthermia, and a sub-range of temperatures ≥39.0° C. which indicates critical threshold temperatures during pregnancy.

The sub-range of follicular phase user-specific temperatures may be a range defined by a basal temperature of the user in the follicular phase of their menstrual cycle, the basal temperature learnt according to one or more embodiments of the present invention. The sub-range of luteal phase user-specific temperatures may be a range defined by a basal temperature of the user in the luteal phase of their menstrual cycle, the basal temperature learnt according to one or more embodiments of the present invention. Typically, a user's basal temperature during the luteal phase of their menstrual cycle is 0.2-0.3° C. higher than their basal temperature during the follicular phase of their menstrual cycle.

For the women's health use case, the notifications associated with the sub-range of follicular phase-user specific temperatures may include a notification that the user's temperature has returned to their baseline temperature. The notifications associated with the sub-range of luteal phase user-specific temperatures may include a notification to the user that their temperature has slightly increased, a notification to the user that they may have ovulated, and a prompt for the user to input whether they are experiencing any symptoms. The notifications associated with the sub-range of >37.7° C. to <38.0° C. may include a notification to the user that their temperature is slightly elevated, a prompt to the user to input whether they are experiencing any other symptoms, and a prompt for the user to continue monitoring their temperature. The notifications associated with the sub-range of ≥38.0° C. may include a notification to the user that their temperature is elevated, a prompt for the user to input whether they are experiencing any other symptoms, a prompt for the user to consume water, and a prompt for the user to continue to monitor their temperature and to seek medical attention if their temperature remains elevated or increases. The notifications associated with the sub-range of ≥39.0° C. may include a prompt for the user to continue to monitor their temperature or a prompt for the user to seek medical attention promptly.

The selected notification may be output to the user in real time.

In this way, a user may be provided with actionable feedback which may mitigate adverse effects and drive safe choices. Further, the user may be informed of their current clinical attributes.

The computer-implemented method outputting notifications may be selectable by the user. For example, a user may turn off notifications. This may be useful, for example, if the user is conducting an activity which they know will affect their body temperature such as exercising.

The notification data table may associate notification with stored other sensor information. The notification data table may associate notification with stored user input information. The computer-implemented method may comprise comparing acquired other sensor information with stored other sensor information. Selecting the notification to output to the user may be further based upon this comparison. The computer-implemented method may comprise comparing acquired user input information with stored user input information. Selecting the notification to output to the user may be further based upon this comparison.

In this way, the notification selected may be more effective at improving the clinical physiological status of the user. Further, the notification selected may provide a more accurate indication of the clinical attribute of the user, by taking into account other clinically relevant information.

For example, if a temperature is derived which is higher than a basal temperature, and user input/other sensor information indicates that the user is hydrated and has not conducted physical activity, the selected notification may indicate to the user that they may have an illness or infection.

As a further example, the prompt for the user to move around may be selected if the other sensor information and/or the user input information indicates that the user is currently inactive. The prompt for the user to put on additional clothing, to drink warm fluids, or to move to a warmer location may be selected if the other sensor information and/or the user input information indicates that the user is currently inactive. The prompt for the user to move to a warmer location may be selected if the other sensor information and/or the user input information indicates that the user is currently in a cool climate. The prompt for the user to continue to monitor their temperature and consume fluids may be selected if the other sensor information and/or user input information indicates that the user is inactive. Further, the prompt for the user to continue to monitor their temperature and consume fluids may be selected if a subsequent derived temperature indicates that the user's temperature remains elevated. The prompt for the user to consume electrolyte fluid or water may be selected if the other sensor information and/or user input information indicates that the user is dehydrated. For example, the prompt for the user to consume electrolyte fluid or water may be selected if a hydration rank derived in accordance with one or more embodiments of the present invention indicates that the user is dehydrated. The prompt for the user to consume electrolyte fluid or water may be selected if hydration information obtained in accordance with one or more embodiments of the present invention indicates that the user is dehydrated. The prompt for the user to input whether they are experiencing any symptoms may be selected after a duration equal, to a predicted duration of the user's menstrual cycle. The prompt for the user to input whether they are experiencing any symptoms may be selected if the other sensor information and/or user input information indicates that the user is inactive.

In this way, the derived physiological rank may be combined with other sensor information and/or user input information to determine a notification to output to the user.

The computer-implemented method may comprise applying a user-notification machine learning model to the physiological rank. The user-notification machine learning model may take as an input the physiological rank. The user-notification machine learning model may output a notification based on the physiological rank.

The user-notification machine learning model may have been trained using training data. The training data may comprise a plurality of physiological ranks and a respective plurality of context labels. Each of the context labels may comprise user input information and/or other sensor information corresponding to the respective physiological rank.

In this way, physiological ranks of a user can be correlated with clinical data which indicates characteristics and/or behaviors of the user.

The training data may have been acquired from a plurality of subjects.

The training data may have been acquired from a single subject. The single subject may be the user of the wearable device.

The computer-implemented method may comprise applying the user-notification machine learning model to the physiological rank and to the user input information and/or the other sensor information. The user-notification machine learning model may take as inputs the physiological rank and the user input information and/or the other sensor information. The user-notification machine learning model may output a notification based on the physiological rank and the user input information and/or the other sensor information.

For example, the user-notification machine learning model may select a notification to output to the user based upon a derived temperature of the user and a user input indicative of where the user is in their menstrual cycle, such as their dates of menstruation or their cervical mucous state. For example, the notification may indicate that the user may have ovulated if this is indicated by the derived temperature and the user input.

Further, for example, the machine learning model may select a notification

which notifies the user that they may be pregnant if the derived temperature and the user input and/or other sensor information indicate that the user may be pregnant. For example, the basal temperature of a user when they are pregnant may be approximately 0.2-0.3° C. higher than the basal temperature of the user when they are not pregnant.

The computer-implemented method may comprise storing the derived physiological rank and storing the time at which the physiological rank is derived. The computer-implemented method may comprise obtaining time-correlated physiological rank information from previously derived stored physiological ranks and their corresponding stored times. The computer-implemented method may comprise outputting the time-correlated physiological rank information to the user such that the user can track how their physiological rank varies over time.

In this way, an output may be provided which demonstrates the variation of the clinical physiological status of the user over time.

The computer-implemented method may comprise storing the other sensor information and/or user input information. The computer-implemented method may comprise obtaining time-correlated other sensor information and/or time correlated user input information from previously derived stored other sensor information and/or stored user input information and their corresponding stored times. The computer-implemented method may comprise outputting the time-correlated other sensor information and/or time-correlated user input information to the user such that the user can track how they vary over time.

The computer-implemented method may comprise using the time-correlated physiological rank information and the time-correlated other sensor information and/or user input information to correlate the physiological ranks with the other sensor information and/or user input information.

In this way, an output may be provided which demonstrates the impact of clinical factors on the clinical physiological status of the user.

In this way, for example, a user's temperature can be tracked over time to show changes and correlations with other factors such as hydration and concentration of metabolic fuels.

The computer-implemented method may comprise outputting an output to the user. The output may be output to the user in real time. The output may be output to the user at predetermined time points, for example the output may be output to the user every hour, every 6 hours, every 12 hours or every 24 hours. The output may be output to the user upon receiving a user input signal. Outputting the output to the user may mean displaying the output on the wearable device. Outputting the output to the user may mean displaying the output on an external device, which may be a mobile device such as a mobile phone.

Whether the output is output in real time or at pre-determined time points may depend upon the use case of the computer-implemented method.

In this way, information may be provided to the user in a more accessible way than can be provided by laboratory tests.

The output may include the physiological rank.

The computer-implemented method may comprise deriving a time-averaged physiological rank.

The output may include the time-averaged physiological rank. In this way, a more accurate indication of the physiological status of the user may be provided.

The output may include the selected recommendation.

The output may include the time-correlated physiological rank information. The output may include the time-correlated other sensor information and/or the time correlated user input information.

The output may include a derived temperature. The output may include a confidence value of the derived temperature, which may be a standard deviation of the derived temperature.

The derived temperature may be output in units of degree Celsius or degrees Fahrenheit.

In a second aspect, one or more embodiments of the invention provide a computer-implemented method for deriving a physiological rank indicative of a physiological status of a user, the computer-implemented method comprising applying a model to bodily parameter data acquired from a user to obtain physiological information related to the user, and deriving, from the physiological information, a physiological rank indicative of a physiological status of the user wearing the device, wherein the physiological rank is a value on a physiological rank scale.

The computer-implemented invention may include any one or any combination insofar as they are compatible of the features of the computer-implemented method according to the first aspect of the invention.

In a third aspect, one or more embodiments of the invention provide a computer program which when executed causes one or more processors to perform the method according to the first aspect or the second aspect of the invention.

The one or more processors may be components of the wearable device. The one or more processors may be components of a device external to the wearable device, for example a mobile device such as a mobile phone.

In a fourth aspect, one or more embodiments of the invention provide a method for determining a physiological status of a user, the method comprising providing an optical sensing module on a wearable device worn by a user; and providing a processor; and carrying out, by the processor, the computer-implemented method according to the first aspect of the invention, wherein the sensor is the optical sensing module on the wearable device.

In a fifth aspect, one or more embodiments of the invention provide a device comprising a processor, the processor configured to carry out the computer-implemented method according to the first or the second aspect of the invention.

The device may comprise a storage medium storing the computer program according to any one or more embodiments of the present invention

The device may be the wearable device.

The device may comprise the optical sensing module according to any one or more embodiments of the present invention.

The device may be a mobile device such as a mobile phone or tablet.

In a sixth aspect, embodiments of the present invention provide a computer-implemented method for determining a hydration status of a user, the computer-implemented method comprising acquiring, from sensor on a wearable device worn by a user, data including bodily parameter data (for example in the form of an absorption spectrum) related to the user, and applying a model to the bodily parameter data to obtain hydration information related to the user, wherein the model derives, from the hydration information, a hydration rank indicative of a hydration status of the user, wherein the hydration rank is a given grade on a hydration rank scale.

Thus, an indication of the hydration status of a user can be derived from wearable device-acquired data. In this way, an indication of the hydration status of the user may be provided during a user's normal routine. Advantageously then, an indication of the hydration status of the user may be provided in a more accessible way than can be provided by laboratory tests.

Furthermore, daily water intake by the user may be improved and the risk of dehydration decreased. Cognitive and physical performance may be improved as a result. Use of the methods and devices described herein may therefore contribute to: decreased rates of hospital admission due to dehydration and/or overhydration; potential decreases in hospital costs such as IV fluids, laboratory tests, staffing and related fees; improvements in symptoms related with anxiety, depression and/or PTSD; decreased risk of thermal injury such as accidental hyperthermia; decreased risk of kidney stones; improved dialysis and diuresis treatments; decreased risk of hypovolemia or volume depletion; decreased risk of hypotension; optimized dietary fluid restriction or similar; optimized fluid administration and/or improved survival rates during shock and trauma.

Optional features of the computer-implemented method will now be described. The computer-implemented method may have any one, or any combination insofar as they are compatible, of the following features.

The computer-implemented method may be carried out on the wearable device, or on an external device. The external device may be a mobile device such as a mobile phone. The wearable device may be any device worn on the body, for example a watch, a ring, a necklace, bracelet, an ear bud, a skin contact patch, a glasses frame, or a strap worn around the wrist, the arm, the leg, or the torso.

The hydration status of the user may be a clinical categorization, or a clinical hydration status. The hydration status of the user may be hypovolemia, euvolemia, or hypervolemia. The hydration status of the user may be severe hypovolemia, moderate hypovolemia, mild hypovolemia, euvolemia, mild hypervolemia, moderate hypervolemia or severe hypervolemia. A hydration status of hypovolemia may include hydration statuses of mild hypovolemia, moderate hypovolemia and severe hypovolemia. A hydration status of hypervolemia may include hydration statuses of mild hypervolemia, moderate hypervolemia and severe hypervolemia.

Hypovolemia may be referred to as dehydration. Hypervolemia may be referred to as overhydration. Euvolemia may be referred to as normal hydration. Mild hypovolemia, euvolemia and mild hypervolemia may be referred to as normal hydration.

The bodily parameter data may be an optical spectrum. The bodily parameter data may be an absorption spectrum. The bodily parameter data may be a body tissue absorption spectrum. The absorption may be in the water band.

The hydration information may be a quantitative value associated with the bodily parameter data. The hydration information may be a quantitative value associated with the absorption spectrum. For example, the hydration information may be a position of a peak of the absorption spectrum, for example a wavelength shift of a peak of the absorption spectrum, a height of a peak of the absorption spectrum, or a width of a peak of the absorption spectrum.

Each hydration rank on the hydration rank scale may map onto a respective output of a standard clinical point of care test. Each hydration rank on the hydration rank scale may map onto a respective range of outputs of a standard clinical point of care test.

In this way, a hydration rank may be derived which provides a clinically relevant assessment of hydration status.

The standard clinical point of care test may be a body mass measurement, a test performed on a urine sample, or a test performed on a blood sample. The standard clinical point of care test may be a test of urine osmolality (UO), urine specific gravity (USG), fluid gain or weight gain, fluid loss of weight loss, or serum osmolality (SO). The standard clinical point of care test may be a test of temperature. The standard clinical point of care test may be a test of heart rate. The standard clinical point of care test may be a test of urine color, a test of urine volume, a test of skin turgor, a test of jugular venous distention, or an ultrasound test. The standard clinical point of care test may define ranges of outputs of the standard clinical point of care test, wherein each of the ranges corresponds to a respective clinical hydration status.

An output of a UO test may have units of mOsm/kg. An output of a UO test which is <80 may indicate overhydration. An output of a UO test which is <500 may indicate overhydration. An output of a UO test which is >80 to <500 may indicate overhydration, with values below 80 mOsm/kg considered critical. An output of a UO test which is between approximately 500 and 700 may indicate normal hydration. An output of a UO test which is between approximately 700 and >1200 may indicate dehydration.

An output of a USG test which is within the range 1.01 to <1.005 may indicate overhydration. An output of a USG test which is within the range 1.005 to <1.020 may indicate normal hydration. An output of a USG test which is within the range 1.020 to 1.040 may indicate dehydration.

An output of an SO test may have units of mOsm/kg. An output of an SO test which is <265 may indicate overhydration. An output of an SO test which is <285 may indicate overhydration. An output of an SO test which is >265 to <285 may indicate overhydration. An output of an SO test which is within the range 285 to <295 may indicate normal hydration. An output of an SO test which is within the range 295 to 320 may indicate dehydration.

An output of a weight loss test may have units of % of body mass. An output of a weight loss test which is weight gain indicates overhydration. An output of a weight loss test which is within the range 0% to <2% may indicate normal hydration. An output of a weight loss test which is within the range 2% to <6% may indicate mild dehydration. An output of a weight loss test which is within the range 6% to <10% may indicate moderate dehydration. An output of a weight loss test which is greater than or equal to 10% may indicate severe dehydration.

Each hydration rank on the hydration rank scale may map onto respective outputs of a plurality of standard clinical point of care tests. For example, a hydration rank may map onto an output of a urine osmolality test and an output of a urine specific gravity test. Each hydration rank on the hydration rank scale may map onto respective ranges of outputs of a plurality of standard clinical point of care tests.

The plurality of standard clinical point of care tests may include two or more of a test of urine osmolality (UO), a test of urine specific gravity (USG), a test of fluid gain or weight gain, a test of fluid loss or weight loss, a test of serum osmolality (SO), a test of temperature, a test of heart rate, a test of urine color, a test of urine volume, a test of skin turgor, a test of jugular venous distention, and an ultrasound test.

In this way, each hydration rank on the hydration rank scale may relate to a combination of standard clinical point of care tests. Thus, a more accurate indication of a user hydration status may be provided, because the accuracy of the hydration status indicated by the hydration rank is not limited by the inaccuracies of a single standard point of care test.

The computer-implemented method may comprise outputting an output to the user. The output may be output to the user in real time. The output may be output to the user at predetermined time points, for example the output may be output to the user every 30 minutes, every hour, every 6 hours, every 12 hours or every 24 hours. The output may be output to the user upon receiving a user input signal. Outputting the output to the user may mean displaying the output on the wearable device. Outputting the output to the user may mean displaying the output on an external device, which may be a mobile device such as a mobile phone.

In this way, information may be provided to the user in a more accessible way than can be provided by laboratory tests.

The output may include the hydration rank.

Further, the turnaround time of results in clinical scenarios may be shortened.

The computer-implemented method may comprise deriving a time-averaged hydration rank.

The output may include the time-averaged hydration rank.

In this way, a more accurate indication of the hydration status of the user may be provided.

The computer-implemented method may continuously acquire data. The computer-implemented method may continuously derive the hydration rank. The computer-implemented method may acquire data at pre-determined time points, for example the data may be acquired every 30 minutes, every hour, every 6 hours, every 12 hours or every 24 hours. The computer-implemented method may acquire data upon receiving a user input signal. The computer-implemented method may derive the hydration rank at predetermined time points, for example the hydration rank may be derived every hour, every 6 hours, every 12 hours or every 24 hours. The computer-implemented method may derive the hydration rank upon receiving a user input signal.

The hydration rank may include a hydration index. The hydration index may be a given value on a hydration index scale.

A value may mean a numerical value.

In this way, a hydration index scale may be derived which provides a quantitative assessment of hydration status.

Each hydration index on the hydration index scale may map onto a respective output of the standard clinical point of care test. Each hydration index on the hydration index scale may map onto a respective range of outputs of a standard clinical point of care test.

In this way, a hydration index may be derived which provides a clinically relevant quantitative assessment of hydration status.

In one or more embodiments, the presentation of hydration status to the user may be designed in a way that credibly assists the user. For example, a display may include a color output, where the color is associated with hydration status. Alternatively, or additionally, visual indicators such as arrows may be presented to the user.

The standard clinical point of care test may be a body mass measurement, a test performed on a urine sample, or a test performed on a blood sample. The standard clinical point of care test may be a test of urine osmolality, urine specific gravity, fluid gain or weight gain, fluid loss or weight loss or serum osmolality. The standard clinical point of care test may be a test of temperature. The standard clinical point of care test may be a test of heart rate. The standard clinical point of care test may be a test of urine color, a test of urine volume, a test of skin turgor, a test of jugular venous distention, or an ultrasound test.

Each hydration index of the hydration index scale may map onto respective outputs of a plurality of standard clinical point of care tests. For example, a hydration index may map onto an output of a urine osmolality test and an output of a urine specific gravity test. Each hydration index of the hydration index scale may map onto respective ranges of outputs of a plurality of standard clinical point of care tests.

In this way, each hydration index on the hydration index scale may relate to a combination of standard clinical point of care tests. Thus, a more accurate indication of a user hydration status may be provided, because the accuracy of the hydration status indicated by the hydration index is not limited by the inaccuracies of a single standard point of care test.

The hydration index scale may be sub-divided into a plurality of sub-ranges of hydration index values, each of the plurality of sub-ranges corresponding to a different clinical hydration status of the user. The computer-implemented method may comprise determining which sub-range of the plurality of sub-ranges the hydration index value falls within. The computer-implemented method may comprise determining, based upon the determined sub-range, the clinical hydration status of the user. The computer-implemented method may comprise outputting the clinical hydration status of the user, in addition or alternatively, the computer-implemented method may comprise outputting the hydration index.

Each index value on the hydration index scale may be indicative of a clinically-determined hydration status. A clinical hydration status of a user may be determined.

The hydration index scale may consist of hydration index values which are integers. The hydration index scale may comprise any number of hydration indices, for example the hydration index scale may comprise 5, 10, 15 or 20 hydration indices.

The hydration index scale may vary depending upon the spectral resolution of the sensor. The hydration index scale may depend upon the use case. The use case may be selectable by the user.

For example, a hydration index scale for a use case focused on dehydration may consist of negative hydration indices.

The hydration index scale may run from a lower value, e.g., −5, to an upper value, e.g. +5. A hydration index scale which runs from the lower value to the upper value (e.g. −5 to +5) may be applicable to critical care users. Alternatively, it may be applicable to healthy, high risk users such as athletes.

A given hydration index value between the upper and lower value (e.g. a hydration index value of 0) may indicate euvolemia. A sub-range of hydration index values (e.g. a first sub-range including negative hydration index values of e.g. −1, −2, −3, −4 and −5) may indicate dehydration. A smaller sub-range within the first sub-range (e.g. a sub-range including hydration index values of −1 and −2) may indicate mild dehydration. An alternative sub-range also within the first sub-range, but at greater negative values (e.g. a sub-range including hydration index values of −3 and −4) may indicate moderate dehydration. A hydration index value at or near the lower value (e.g. a hydration index of −5) may indicate severe dehydration. A second sub-range including, for example, positive hydration index values of, e.g. +1, +2, +3, +4 and +5 may indicate overhydration. A sub-range within the second sub-range (e.g. including hydration index values of +1 and +2) may indicate mild overhydration. A further sub-range including, for example, hydration index values of greater magnitude (e.g. hydration index values of +3 and +4) may indicate moderate overhydration. A hydration index value at or near the greatest hydration index value (e.g. of +5) may indicate severe overhydration.

A hydration index value, e.g. the lower value of the range (e.g. of −5) may correspond to USG values of over 1.030. A sub-range including hydration index values (e.g. of −3 and −4) may correspond to USG values of between 1.020 and 1.030. A sub-range (e.g. including hydration index values of −1 and −2) may correspond to USG values of approximately 1.020. A sub-range including hydration index values (e.g. of −1, −2, −3, −4 and −5) may correspond to USG values of between 1.020 and 1.040. A sub-range including hydration index values (e.g. of −2, −1, 0, 1 and 2) may correspond to UDG values of between 1.005 and 1.020. A central hydration index value (e.g. of 0) may correspond to USG values of approximately 1.010. A sub-range including hydration index values (e.g. of +1 and +2) may correspond to a USG value of approximately 1.005. A sub-range including hydration index values (e.g. of +1, +2, +3, and +4) may correspond to USG values of between 1.002 and 1.005. A hydration index value (e.g. of +5) may correspond to USG values of less than 1.002.

A hydration index value at the lower end of the range (e.g. of −5) may correspond to UO values of greater than and including 1200. A first sub-range including hydration index values towards the lower half of the range (e.g. of −1, −2, −3 and −4) may correspond to UO values of between 700 and 1200. A smaller sub-range within the first sub-range including hydration index values (e.g. of −1 and −2) may correspond to UO values of between 700 and 850. A central hydration index value (e.g. a hydration value of 0) may correspond to a UO value of approximately 500. A central sub-range including hydration index values (e.g. of −1, 0 and 1) may correspond to UO values of between 500 and 700. A sub-range including hydration index values at the upper half of the range (e.g. of +1, +2, +3, +4 and +5) may correspond to UO values of <500. A hydration index value at or near the upper end of the range (e.g. a hydration index value of +5) may correspond to UO values below 80 or below 50.

A hydration index value at or near the lower end of the range (e.g. a hydration index value of −5) may correspond to SO values of greater than 320. A sub-range including hydration index values (e.g. of −1 and −2) may correspond to SO values between 295 and 300. A sub-range including hydration index values at the lower half of the range (e.g of −1, −2, −3, −4 and −5) may correspond to SO values greater than and including 295. A central hydration index value (e.g. a hydration index value of 0) may correspond to SO values between 280 and 295. A sub-range including hydration index values at the upper half of the range (e.g. hydration index values of +1, +2, +3, +4 and +5) may correspond to SO values less than 280. A hydration index value at or near the upper end of the range (e.g. of +5 may correspond to SO values less than 265).

A hydration index value at or near the lower end of the range (e.g. of −5) may correspond to fluid loss values of greater than and equal to 10%. A sub-range including greater negative hydration index values (e.g. of −3 and −4) may correspond to fluid loss values of approximately between 6% and 10%. A further sub-range including smaller negative hydration index values (e.g. of −1 and −2) may correspond to fluid loss values of approximately between 2% and 6%. A central hydration index value (e.g. a hydration index value of 0) may correspond to fluid loss values of approximately between 0% and 2%. A sub-range including hydration index values of +1, +2, +3, +4 and +5 may correspond to SO values which indicate weight gain.

A central sub-range (e.g. including hydration index values at either side of and including the mid-point of the range e.g. of −1, 0 and +1) may indicate euvolemia. A sub-range including negative hydration index values of greater magnitude (e.g. hydration index values of −2, −3, −4 and −5) may indicate dehydration. A sub-range including negative hydration index values of a lower magnitude (e.g. −2 and −3) may indicate mild dehydration. A hydration index value of medium magnitude (e.g. −4) may indicate moderate dehydration. A hydration index value at the lower end of the range (i.e. a negative value of greatest magnitude, e.g. of −5) may indicate severe dehydration. A sub-range including hydration index values (e.g. of positive values such as +2, +3, +4 and +5) may indicate overhydration. A hydration index value of greatest magnitude at the upper end of the range (e.g. a hydration index value of +5) may indicate water intoxication.

A hydration index value at or near the lower end of the range (e.g. a hydration index value of −5) may correspond to USG values of over 1.030. A sub-range including hydration index values in the lower half of the range (e.g. negative hydration index values with lower magnitudes of e.g. −2, −3 and −4) may correspond to USG values of approximately 1.020. A sub-range including hydration index value of medium magnitude (e.g. −4) may correspond to USG values of between 1.020 and 1.030. A sub-range including central hydration index values (e.g. values either side of and including the mid-point of the range, e.g. hydration index values of −1, 0, 1) may correspond to USG values of approximately 1.010. A sub-range including hydration index values e.g. of −4, −3, −2, −1, 0, 1, 2, 3, 4 may correspond to USG values of between 1.005 and 1.020. A sub-range including hydration index values of values in the upper half of the range but not including the upper end of the range (e.g. +2, +3, +4) may correspond to a USG value of approximately 1.005. A hydration index value at the upper end of the range (e.g. of +5) may correspond to USG values of less than 1.002.

A hydration index value at the lower end of the range (e.g. of −5) may correspond to UO values of greater than and including 1200. A sub-range at the lower half of the range but not including the lowest end of the range (e.g. including hydration index values of −2, −3 and −4) may correspond to UO values of between 700 and 1200. A sub-range including hydration index values of e.g. −2 and −3 may correspond to UO values of between 700 and 850. A hydration index value at the mid-point of the range (e.g. a hydration index value of 0) may correspond to a UO value of approximately 500. A sub-range including central hydration index values (e.g. values either side of and including the mid-point of the range e.g. values of −1, 0 and 1) may correspond to UO values of between 500 and 700, or between 500 and 650. A sub-range including hydration index values at the upper half of the range (e.g. of +2, +3, +4 and +5) may correspond to UO values of <500. A sub-range including hydration index values at the upper half of the range, but not including the upper end of the range (e.g. values of +2, +3, and +4) may correspond to UO values of between 80 and 500. A hydration index value at or near the upper end of the range (e.g. of +5) may correspond to UO values below 80 or below 50.

A hydration index value at or near the lower end of the range (e.g. a value of −5) may correspond to SO values of greater than 320. A sub-range including hydration index values at the lower end of the range but not including the lowest end value of the range (e.g. values of −2, −3 and −4) may correspond to SO values between 295 and 300. A sub-range including hydration index values e.g. of −2 and −3 may correspond to SO values greater than and including 295 and less than 300. A sub-range including central hydration index values either side of and including the mid-point of the range (e.g. of −1, 0 and 1) may correspond to SO values between 280 and 295. A sub-range of values at the upper end of the range, e.g. including hydration index values of +2, +3, +4 and +5 may correspond to SO values less than 280. A hydration index value at the upper end value (e.g. of +5) may correspond to SO values less than 265.

A hydration index value at the lower end of the range (e.g. of −5) may correspond to fluid loss values of greater than and equal to 10%. A sub-range including a hydration index value in the lower half of the range but not at the lowest endpoint of the range (e.g. −4) may correspond to fluid loss values of approximately between 6% and 10%. A sub-range including hydration index values of e.g. −2 and −3 may correspond to fluid loss values of approximately between 2% and 6%. A sub-range including central hydration index values including values either side of and including the mid-point of the range (e.g. of −1, 0 and 1) may correspond to fluid loss values of approximately between 0% and 2%. A sub-range including hydration index values at the upper half of the range (e.g. of +2, +3, +4 and +5) may correspond to SO values which indicate weight gain.

The hydration index scale may run from 0 to −9. A hydration index scale which runs from 0 to −9 may be applicable to healthy and vulnerable users.

A sub-range including hydration index values at the upper end of the range (e.g. of 0, −1 and −2) may indicate euvolemia. A sub-range including hydration index values at the mid and lower regions of the range (e.g. hydration index values of −3, −4, −5, −6, −7, −8 and −9) may indicate dehydration. A sub-range including hydration index values at the middle of the range, including the mid-point of the range and values either side of the mid-point of the range (e.g of −3, −4, −5, and −6) may indicate mild dehydration. A sub-range including hydration index values at the lower end of the range (e.g. of −7, −8 and −9) may indicate moderate dehydration.

A sub-range at the lower end of the range (e.g. including hydration index values of −7, −8 and −9) may correspond to USG values of between 1.020 and 1.030. A sub-range including hydration index values at and either side of the mid-point of the range (e.g. of −3, −4, −5 and −6) may correspond to USG values of approximately 1.020. A sub-range including hydration index values at the upper end of the range (e.g. of 0, −1 and −2) may correspond to USG values of approximately 1.010. A sub-range including hydration index values at the upper end of the range (e.g. of 0, −1 and −2) may correspond to USG values of between 1.010 and 1.020.

A sub-range including hydration index values at the lower end of the range (e.g. of −7, −8, −9) may correspond to UO values of approximately 1100. A sub-range including hydration index values at and either side of the mid-point of the range (e.g. of −3, −4, −5, and −6) may correspond to UO values of between 700 and 850. A hydration index at the upper end point of the range (e.g. of 0) may correspond to UO values of approximately 500. A hydration index having a value towards the upper endpoint but not at the upper endpoint of the range (e.g. of −1) may correspond to UO values of approximately 650. A hydration index of a lower value, e.g., −2, may correspond to UO values of approximately 700.

A sub-range including hydration index values at the lower end including the lower endpoint of the range (e.g. hydration index values of −7, −8, and −9) may correspond to SO values of above 300. A sub-range including hydration index values at and either side of the mid-point of the range (e.g. of −3, −4, −5, −6) may correspond to SO values between 295 and 300. A sub-range including hydration index values at the upper end of the range including the upper endpoint of the range (e.g. of 0, −1 and −2) may correspond to SO values of between 280 and 295. A hydration index at the upper endpoint of the range (e.g. of 0) may correspond to an SO value of approximately 280. A hydration index of e.g. −2 may correspond to an SO values of approximately 295.

A sub-range including hydration index values at the lower end of the range and including the lowest endpoint of the range (e.g. of −7, −8 and −9) may correspond to fluid loss values of approximately between 6% and 9%. A sub-range including hydration index values including the mid-point of the range and values either side of the mid-point, e.g. of −3, −4, −5, and −6 may correspond to fluid loss values of approximately between 2% and 6%. A sub-range including hydration index values at the upper end of the range and including the upper endpoint of the range (e.g. of 0, −1, and −2) may correspond to fluid loss values of approximately between 0 and 2%.

The hydration rank may include a hydration status of the user, wherein the hydration status is a given grade on a hydration status scale.

Each hydration status on the hydration status scale may be a clinical hydration status. In this way, a clinical hydration status of a user may be determined or indicated.

Each hydration status on the hydration status scale may map onto a respective output of a standard clinical point of care test. Each hydration status on the hydration status scale may map onto a respective range of outputs of a standard clinical point of care test.

In this way, a clinically relevant qualitative assessment of hydration status, or a clinical hydration status, may be provided.

Each hydration status on the hydration status scale may map onto respective outputs of a plurality of standard clinical point of care tests. Each hydration status on the hydration status scale may map onto respective ranges of outputs of a plurality of standard clinical point of care tests.

In this way, each hydration status on the hydration status scale may relate to a combination of standard clinical point of care tests. Thus, a more accurate indication of a user clinical hydration status may be provided, because the accuracy of the derived clinical hydration status, as compared to the users actual hydration status, is not limited by the inaccuracies of a single standard point of care test.

A clinical hydration status of overhydration may correspond to UO values of <500 or >80 and <500. A clinical hydration status of normal hydration may correspond to UO values of 500 to 700. A clinical hydration status of dehydration may correspond to UO values of 700 to 1200.

A clinical hydration status of overhydration may correspond to USG values of 1.001 to <1.005. A clinical hydration status of normal hydration may correspond to USG values of 1.005 to <1.020. A clinical hydration status of dehydration may correspond to USG values of 1.020 to 1.040.

A clinical hydration status of overhydration may correspond to SO values of >265 and <285. A clinical hydration status of normal hydration may correspond to SO values of 285 to <295. A clinical hydration status of dehydration may correspond to SO values of 295 to >320.

A clinical hydration status of overhydration may correspond to body mass measurement of weight gain. A clinical hydration status of normal hydration may correspond to a body mass measurement of 0% to <2% weight loss. A clinical hydration status of mild dehydration may correspond to a body mass measurement of 2% to <6% weight loss. A clinical hydration status of moderate dehydration may correspond to a body mass measurement of 6% to <10% weight loss. A clinical hydration status of severe dehydration may correspond to a body mass measurement of >10%.

The hydration rank may include both the hydration index and the hydration status of the user. The model may include an index model which derives the hydration index, and a status model which derives the hydration status.

The sensor may be an optical sensing module (i.e. an optical sensor) on the wearable device.

In this way, an indication of clinical hydration status may be derived using an optical measurement. Further, an indication of the hydration status of the user may be provided non-invasively. An indication of the hydration status of the user may be provided without requiring a sample to be taken from the user.

The optical sensing module may comprise a laser. The optical sensing module may comprise a plurality of lasers. Each laser of the plurality of lasers may operate at a wavelength that is different from the wavelength of the others. The optical sensing module may be configured to operate each laser one at a time. The optical sensing module may be configured to operate the plurality of lasers in a cycle according to a pre-determined schedule.

In this way, the optical sensing module may require fewer detectors. The optical sensing module may require only one detector. Advantageously then, the optical sensing module may be cheaper and simpler to manufacture than an optical sensing module which requires more detectors.

In one or more embodiments, the optical sensing module may have a sampling rate of 50,000 samples per second or fewer. In one or more embodiments, the sampling rate may be 1,000 samples per second or more. The data acquired from the optical sensing module may be an average of the samples. A laser on-time for the laser or for each of the plurality of lasers be 200 microseconds or more. As an example, the number of samples acquired in 200 microseconds may be 10. Two samples may be discarded in the processing. An example of a laser off-time for the laser or for each of the plurality of lasers may be 100 microseconds. As an example, the number of samples acquired in 100 microseconds may be 5. As an example, the time to perform 60 cycles may be 20 milliseconds. In one or more embodiments, the number of samples acquired in 20 milliseconds may be 1000 or more. A total measurement time may be 10 seconds or less. As an example, the number of cycles in 10 seconds may be 500. A plurality of total measurements may be taken. There may be an interval of 15 seconds between each total measurement.

The laser or the plurality of lasers may emit light in a wavelength band which is sensitive to changes in water concentration within the interstitial space. The laser or the plurality of lasers may emit light in the wavelength band which covers wavelengths between at least 350 nm and no more than 2500 nm. The laser or the plurality of lasers may emit light in the visible wavelength band. The visible wavelength band may cover wavelengths roughly from 300 nm to 780 nm. The laser or the plurality of lasers may emit light in the infrared wavelength band. The laser or the plurality of lasers may emit light in the near-infrared wavelength band. The near infrared wavelength band may cover wavelengths roughly from 780 nm to 1000 nm. The laser or the plurality of lasers may emit light in the short wavelength infrared wavelength band. The short wavelength infrared wavelength band may cover wavelengths from roughly from 1000 nm to 2500 nm. The laser or a laser within the plurality of lasers may emit light at e.g. 970 nm, 1200 nm, 1450 nm, 1950 nm, 2766 nm, 2898 nm, or 6097 nm.

The optical sensing module may comprise one or more optical outputs for light originating from the laser or the plurality of lasers. Light from the laser or the plurality of lasers may exit the optical sensing module via one or more optical output ports. The optical sensing module may comprise a mirror to take the light from the plane of the optical sensing module and translate it into a direction more suitable for interrogating the surface. The direction may be orthogonal or substantially orthogonal to the plane of the optical sensing module.

The optical sensing module may include a transmitter photonic integrated circuit (PIC). The optical sensing module may comprise a substrate. The substrate may be a silicon substrate. The transmitter PIC may be located on the substrate. The transmitter PIC may include the laser or the plurality of lasers. The transmitter photonic integrated circuit (PIC) may be a silicon or silicon nitride photonic integrated circuit.

In use, light emitted from a laser of the optical sensing module may reflect or backscatter from a layer of the skin of the user. The optical sensing module may be configured to receive light backscattered from the skin of the user.

The optical sensing module may comprise a detector. The detector may be a photodetector. The detector may be located on the transmitter PIC such that the PIC is a transmitter/receiver PIC. The detector may be located separately from the transmitter PIC. The photodetector may be a silicon-based photodetector. The photodetector may be an InGaAs-based photodetector. The photodetector may be a germanium photodetector. The photodetector may be located on a receiver PIC that is vertically integrated and mounted on the same substrate as the transmitter PIC. The optical sensing module may comprise a plurality of detectors.

The optical sensing module may comprise an optical manipulation region. The optical manipulation region may comprise one or more of an optical modulator, an optical multiplexer, and additional optical manipulation elements.

The optical sensing module may be that disclosed in WO 2021/116766, the contents of which are incorporated herein by reference in its entirety.

In this way, the optical sensing module may have the ability to continuously take data. Therefore, the computer-implemented method may continuously receive data. Therefore, the hydration status of the user may be continuously monitored. Further, the hydration rank of the user may be provided in real time.

The optical sensing module may comprise LEDs instead of lasers. LEDs may cheaper and simpler to manufacture than lasers. Lasers may allow for a more accurate indication of the hydration status of the user.

The bodily parameter data may be a body tissue absorption spectrum. The absorption may be in the water band. In some examples, the wavelength of the laser or a laser in the plurality of lasers may correspond to the wavelength of a water absorption peak.

In this way, there may be provided a more direct indication of the hydration status of the user, as compared to standard clinical tests which measure proxies for hydration status. Further, a more accurate indication of hydration status of the user may be provided.

The model may include a machine learning model. The model may include a regression model. The model may include a classifier. The model may include a logistic regression model. The model may include a partial least squares (PLS) regression model. The model may include a principal component analysis (PCA) model. The PCA model may be applied before the regression model. The PCA model may be applied before the classifier.

The model may have been trained using training data. The training data may include a plurality of training datasets, each of the training datasets comprising bodily parameter data, and each of the training datasets acquired form the wearable device. The training data may include clinical labels. Each training dataset may be associated with a respective one of the clinical labels. Each of the clinical labels may include an output of a standard clinical point of care test. The clinical data which is used to derive the output of the standard clinical point of care test may have been acquired at the same time as or at a similar time to the acquisition of the corresponding training dataset, for example within the same 5 minute interval, 15 minute interval, or 1 hour interval. Each of the clinical labels may include a plurality of outputs of a respective plurality of standard clinical point of care tests.

The model trained or generated in this way is not limited to a machine learning model.

The model may include an offline model. The offline model may have been trained using batch training data.

The training data may have been acquired from a single subject. The single subject may be the user of the wearable device. The training data may have been acquired from a plurality of subjects.

The model may comprise one or more pre-processing steps. The one or more pre-processing steps may comprise applying a statistical model to the data acquired from the sensor to validate the data acquired from the sensor.

In one or more embodiments, the computer-implemented method may comprise a step of detecting whether the bodily parameter data has been acquired from a human body.

Validating the data may comprise determining whether the data has been acquired from a human user. In addition, or alternatively, validating the data may comprise determining whether the data is anomalous data.

In this way, the effect of any anomalous data, or any incorrectly acquired data on the accuracy of the hydration status indication is reduced.

The statistical model may be generated using a plurality of training datasets, each dataset comprising bodily parameter data. The plurality of training datasets may have been acquired from a plurality of subjects. To validate the data acquired from the sensor, the statistical model may determine whether the data acquired from the sensor falls within a pre-determined number of standard deviations, for example within 2 standard deviations, of the plurality of training datasets. To validate the data acquired from the sensor, the statistical model may calculate a Mahalonobis distance metric between the data acquired form the sensor and the plurality of training datasets, and determine whether the Mahalonobis distance metric is within a given threshold.

The one or more pre-processing steps may comprise applying a baseline correction to the data. Applying the baseline correction to the data may comprise subtracting baseline data from the acquired data. The baseline data may be derived from data previously acquired from the user. The baseline data may be average data acquired from the user. The average data may have been derived from data acquired from the user over a long time period, for example, over 24 hours, 1 week, or 1 month.

In this way, the effect of noise in the data may be reduced. In this way, the indication of the user's hydration status may be more accurate.

The computer-implemented method may comprise acquiring other sensor information in addition to the hydration information. The computer-implemented method may comprise acquiring user input information.

The other sensor information may be acquired from the sensor on the wearable device. The other sensor information may be acquired from an additional sensor. The additional sensor may be external to the wearable device. The additional sensor may be on the wearable device. The other sensor information may be obtained from other bodily parameter data related to the user. The other bodily parameter data may be an optical spectrum.

The other sensor information may include clinically relevant information. The other sensor information may include one or more of body temperature information obtained from a temperature sensor, heart rate information obtained from a heart rate sensor, blood oxygen saturation information obtained from a blood oxygen saturation sensor, respiratory rate information obtained from a respiratory rate sensor, hydration information obtained from a hydration sensor, accelerometer and motion information obtained from an accelerometer or a motion sensor, heart rate variability information obtained from a heart rate sensor, alcohol concentration, sleep/wake information obtained from a sleep sensor, blood pressure information obtained from a blood pressure sensor, and analyte concentration information (wherein the analyte may be a metabolic fuel such as glucose, lactate, or ethanol) obtained from an analyte concentration sensor.

The heart rate sensor, the blood oxygen saturation sensor and the respiration rate sensor may be a PPG sensor. The blood pressure sensor and the heart rate sensor may be an SPG sensor. The temperature sensor may be a short-wavelength infrared sensor. The heart rate information and the heart rate variability information may be obtained from an electrocardiogram.

The other sensor information may be or may be measured using physiological indicators. Physiological indicators may include tissue perfusion or ischemia, infection, decompensation, pain, performance, overtraining, movement/activity, core body temperature, resting heart rate, real-time heart rate, maximum heart rate, heart rate thresholds, VO₂ or VO₂ maximum, intensity, sleep quality, sleep disturbance, apnea hypopnea index (AHI), oxygen desaturation index (ODI), metabolic equivalent of tasks (METs), metabolic health, caloric cost, or general health status.

For example, the blood oxygen saturation information may be an oxygen desaturation index. The heart rate information may be a VO₂ measurement or a maximum VO₂ measurement. The heart rate information may be an MET measurement. The respiration rate information may be an apnea-hypopnea index or a respiratory disturbance index.

The user input information may be acquired from a user input into the wearable device. The user input information may be acquired from a user input into an external device which may be a mobile device such as a mobile phone. The user input information may include one or more of weight information, height information, activity information, diet information, fluid intake information, sodium intake information, illness information, intoxication information and blood pressure information.

The user input information may include a value on a clinically relevant scale. For example, weight information may include a mass in kg, or pounds. Weight information may include a body mass index (BMI). Height information may include a height in cm or inches. Activity information may include an amount of calories burnt. Activity information may include information from which an amount of calories burnt could be calculated, for example a type of exercise and a duration of the exercise. Activity information may also include duration of an activity completed, type of activity completed or other information related to the user's experience of the activity, such as perceived exertion. Diet information may include a food group (such as fat, carbohydrate or protein) consumed. Diet information may include an amount of calories consumed. Diet information may include information from which an amount of calories consumed could be calculated, for example a type of food and an amount of food. Fluid intake information may include a volume of fluid consumed. Fluid intake information may include a type of fluid consumed, for example water or an electrolyte fluid. Illness information may include a temperature. Illness information may include a type of diagnosed illness, a duration of illness or other information related to symptoms. Intoxication information may include a number of alcohol units consumed. Intoxication information may include information from which a number of alcohol units consumed could be calculated, for example a type of alcohol and a volume consumed. Intoxication information may include a number of days in which alcohol has been consumed. Blood pressure information may include a measurement in mmHg. Blood pressure information may include a ratio of systolic pressure to diastolic pressure, where each pressure may be a measurement in mmHg.

The computer-implemented method may comprise acquiring a learnt basal hydration rank of the user or a learnt basal bodily parameter data of the user. Basal bodily parameter data may mean average bodily parameter data over a prolonged period for example over 24 hours, 1 week or 1 month. Basal bodily parameter data may be bodily parameter data acquired when the user is at rest. A basal hydration rank may mean an average hydration rank over a prolonged period for example over 24 hours, 1 week or 1 month. A basal hydration rank may be derived from data acquired when the user is at rest, the data including basal bodily parameter data.

The learnt basal hydration rank or basal bodily parameter data may be acquired from a memory. The memory may be located in the wearable device or in an external device which may be a mobile device such as a mobile phone.

The computer-implemented method may further comprise applying a basal hydration model to the bodily parameter data. The basal hydration model may take the learnt basal bodily parameter data as an input. The basal hydration model may derive whether the bodily parameter data is a pre-determined threshold away from the basal bodily parameter data of the user.

The computer-implemented method may further comprise applying a basal hydration model to the hydration rank. The basal hydration model may take the learnt hydration rank as an input. The basal hydration model may compare the derived hydration rank and the basal hydration rank and, based on this comparison, may determine whether the derived hydration rank is a pre-determined threshold away from a basal hydration rank of the user.

The computer-implemented method may comprise, when the basal hydration model determines that the bodily parameter data is more than the pre-determined threshold away from a user's basal bodily parameter data, alerting the user. The computer-implemented method may comprise, when the basal hydration model determines that the derived hydration rank is more than a pre-determined threshold away from a user's basal hydration rank, alerting the user.

The alert may be output by the wearable device. The alert may be output by an external device. The alert may be a haptic, aural or a visual alert. For example, the alert may be a visual indication on the wearable device that the user is out of their basal hydration range.

In this way, a physical output may be provided to the user when the user is out of their basal hydration range.

The learnt basal bodily parameter data or the learnt basal hydration rank may have been learnt using a machine learning model. The learnt basal bodily parameter data or the learnt basal hydration rank may have been learnt in a calibration period of the computer-implemented method. The calibration period may be an initial period in which a user is using the computer-implemented method. The calibration period may be between 1 day and 21 days. The calibration period may be between 7 days and 14 days. The learnt basal bodily parameter data or the learnt basal hydration rank may have been learnt in a plurality of calibration sub-periods within the calibration period. Each calibration sub-period may be between 1 minute and 1 hour. Each calibration sub-period may be between 5 minutes and 15 minutes. Each of the calibration sub-periods may be a period in which the user has just woken up, for example a period in which the user has woken up within the last 5 minutes, 15 minutes, 30 minutes or 1 hour.

The learnt basal bodily parameter data of the user may have been learnt for changing other sensor information and/or for changing user input information. The learnt basal hydration rank of the user may have been learnt for changing other sensor information and/or for changing user input information.

The training data used to learn the basal hydration rank or the basal bodily parameter data may include a plurality of training basal datasets, the training basal datasets including bodily parameter data, and the training basal datasets acquired from the wearable device. The training data may comprise a plurality of context labels. Each training basal dataset may be associated with a respective one of the context labels. Each of the context labels may include training other sensor information and/or training user input information. The training other sensor information may be acquired at the same time as or at a similar time to the acquisition of the corresponding training basal dataset, for example within the same 5 minute interval, 15 minute interval, or 1 hour interval. The training user input information be acquired at the same time as or at a similar time to the acquisition of the corresponding training basal dataset, for example within the same 5 minute interval, 15 minute interval, or 1 hour interval.

The training user input information may include weight information, height information, activity information, diet information, fluid intake information, illness information, intoxication information, or blood pressure information. The training other sensor information may include body temperature obtained from a temperature sensor, activity information obtained from an accelerometer, heart rate information obtained from a heart rate sensor or blood pressure information obtained from a blood pressure sensor.

The parameters being learnt in this way does not limit the model used to learn the parameters to being a machine learning model.

In this way, basal bodily parameter data or a basal hydration rank of a user may be learnt. Further the basal bodily parameter data or the basal hydration rank may be correlated with user input information and/or other sensor information.

In one or more embodiments, the computer-implemented method may comprise storing a hydration status cause data table, the hydration status cause data table associating causes of a clinical hydration status with stored other sensor information and/or stored user input information respectively; and, optionally, when a hydration rank is derived which indicates that the clinical hydration status of the user is a pre-determined clinical hydration status; comparing acquired other sensor information and/or user input information with stored other sensor information and/or stored user input information respectively and, based on this comparison. The computer-implemented method may further comprise a step of selecting a cause of a clinical hydration status, and, optionally, outputting the selected cause of the clinical hydration status to the user.

The computer-implemented method may comprise, when a hydration index is derived which falls within a pre-determined sub-range, comparing acquired other sensor information and/or user input information with stored other sensor information and/or user input information respectively, and based on this comparison, selecting a cause of a clinical hydration status. When a hydration index is derived which falls within a sub-range other than the pre-determined sub-range, the computer-implemented method may not carry out these comparison and selection steps.

The computer-implemented method may comprise outputting the selected cause of the clinical hydration status to the user. The cause of the clinical hydration status may be output to the user in real time.

The pre-determined clinical hydration status may be dehydration. The pre-determined clinical hydration status may be mild dehydration, moderate dehydration or severe dehydration. The pre-determined clinical hydration status may be overhydration. The pre-determined clinical hydration status may be mild overhydration, moderate overhydration or severe overhydration.

The pre-determined sub-range may correspond to a clinical hydration status of dehydration. The pre-determined sub-range may correspond to a clinical hydration status of mild dehydration, moderate dehydration or severe dehydration. The pre-determined sub-range may correspond to a clinical hydration status of overhydration. The pre-determined sub-range may correspond to a clinical hydration status of mild overhydration, moderate overhydration or severe overhydration.

In this way, clinically relevant factors may be taken into account to derive the cause of a clinical hydration status of the user.

A cause of dehydration may include an active cause of dehydration, a passive cause of dehydration or an illness or condition.

The hydration status cause data table may associate causes of a hydration status with types of hydration status. For example, the hydration status cause data table may associate causes of dehydration with types of dehydration. Types of dehydration may include hyopotonic, hypertonic and isotonic. The computer-implemented method may comprise outputting a type of hydration status to the user, where the type of hydration status is associated with the selected cause of the clinical hydration status.

The hydration status cause data table may associate types of hydration status with stored other sensor information and/or stored user input information.

The computer-implemented method may comprise, when a hydration rank is derived which indicates that the clinical hydration status of the user is a pre-determined clinical hydration status, comparing acquired other sensor information and/or user input information with stored other sensor information and/or user input information respectively, and based on this comparison, selecting a type of a clinical hydration status. When a hydration rank is derived which indicates that the user's clinical hydration status is not the pre-determined clinical hydration status, the computer-implemented method may not carry out these comparison and selection steps.

The computer-implemented method may comprise, when a hydration index is derived which falls within a pre-determined sub-range, comparing acquired other sensor information and/or user input information with stored other sensor information and/or user input information respectively, and based on this comparison, selecting a type of a clinical hydration status. When a hydration index is derived which falls within a sub-range other than the pre-determined sub-range, the computer-implemented method may not carry out these comparison and selection steps.

The computer-implemented method may comprise outputting the selected type of the clinical hydration status to the user. The type of the clinical hydration status may be output to the user in real time.

The computer-implemented method may comprise storing a recommendation data table.

The recommendation data table may associate recommendations with stored hydration ranks. The computer-implemented method may comprise comparing the derived hydration rank with the stored hydration ranks and, based on this comparison, selecting a recommendation to output to the user. The computer-implemented method may further comprise outputting the selected recommendation to the user.

The recommendations may be actions for the user to take, for example “drink water” or “stop consuming water”.

The selected recommendation may be output to the user in real time.

The selected recommendation may be one which is clinically understood to improve the hydration status of the user.

In this way, a recommendation appropriate to improving the clinical hydration status of the user may be output to the user. An improved hydration status of the user may be one which is closer to euvolemia.

The recommendation data table may associate recommendations with stored other sensor information. The recommendation data table may associate recommendations with stored user input information. The computer-implemented method may comprise comparing acquired other sensor information with stored other sensor information. Selecting the recommendation to output to the user may be further based upon this comparison. The computer-implemented method may comprise comparing acquired user input information with stored user input information. Selecting the recommendation to output to the user may be further based upon this comparison.

In this way, the recommendation provided to the user may be more effective at improving the clinical hydration status of the user, by taking into account other clinically relevant information.

If the derived hydration rank indicates that the clinical hydration status of the user is severe dehydration, the selected recommendation may include a prompt to ask for help and/or to seek medical attention. If the derived hydration rank indicates that the clinical hydration status of the user is severe dehydration, the selected recommendation may include a prompt for a user to input user-input information about any other symptoms they may have into a device. The device may be the wearable device or an external device.

If the derived hydration rank indicates that the clinical hydration status of the user is mild overhydration, the selected recommendation may include a prompt to stop consuming water and fluids.

If the derived hydration rank indicates that the clinical hydration status of the user is moderate to severe overhydration, the selected recommendation may include a prompt to ask for help and/or to seek medical attention. If the derived hydration rank indicates that the clinical hydration status of the user is moderate to severe overhydration, the selected recommendation may include a prompt to stop consuming water and fluids. If the derived hydration rank indicates that the clinical hydration status of the user is moderate to severe overhydration, the selected recommendation may include a prompt for a user to input user-input information about any other symptoms they may have.

The computer-implemented method may comprise storing a rehydration fluid type data table. The rehydration fluid type data table may associate types of rehydration fluids with stored other sensor information. The rehydration fluid type data table may associate types of rehydration fluids with stored user input information. Types of rehydration fluid may include, for example, water or an electrolyte fluid.

The computer-implemented method may comprise, when a hydration rank is derived which indicates that the clinical hydration status of the user is dehydration, comparing acquired other sensor information with stored other sensor information and, based on this comparison, selecting a type of rehydration fluid, and outputting the selected type of rehydration fluid to the user.

The computer-implemented method may comprise, when a hydration rank is derived which indicates that the clinical hydration status of the user is dehydration, comparing acquired user input information with stored user input information and, based on this comparison, selecting a type of rehydration fluid, and outputting the selected type of rehydration fluid to the user.

When a hydration rank is derived which indicates that the clinical hydration status of the user is not dehydration, the computer-implemented method may not carry out these comparison and selection steps.

The clinical hydration status being dehydration may include the clinical hydration status being mild dehydration, moderate dehydration or severe dehydration.

If the derived hydration rank indicates that the clinical hydration status of the user is mild dehydration, and if the other sensor information and/or user input information indicates that the user has not undergone physical activity and that the user is not under thermal stress, the recommended fluid type may be water. If the derived hydration rank indicates that the clinical hydration status of the user is mild dehydration, and if the other sensor information and/or user input information indicates that the user has undergone physical activity or that the user is under thermal stress, or is undergoing gastrointestinal problems, the recommended fluid type may be electrolyte fluid and/or water.

Gastrointestinal problems may include acute vomiting and/or diarrhea.

If the derived hydration rank indicates that the clinical hydration status of the user is moderate dehydration, and if the other sensor information and/or user input information indicates that the user has not undergone physical activity and that the user is not under thermal stress, the recommended fluid type may be electrolyte fluid and/or water. If the derived hydration rank indicates that the clinical hydration status of the user is moderate dehydration, and if the other sensor information and/or user input information indicates that the user has undergone physical activity, is undergoing gastrointestinal problems, or that the user is under thermal stress, the recommended fluid type may be electrolyte fluid and/or water.

Whether or not the recommended fluid type is an electrolyte fluid may depend upon a user-input of sodium intake.

The rehydration fluid type data table may associate types of rehydration fluids with stored types of dehydration.

The computer-implemented method may comprise comparing a selected type of dehydration selected from the hydration status cause data table with stored types of dehydration and, based on this comparison, selecting a type of rehydration fluid. The computer implemented method may further comprise outputting the selected type of rehydration fluid to the user.

In this way, a rehydration fluid suitable for rehydrating the user, based on data which may indicate a cause of dehydration of the user, may be output to the user.

The computer-implemented method may comprise storing a rehydration fluid volume data table.

The rehydration fluid volume data table may associate volumes of rehydration fluid with stored hydration ranks.

The computer-implemented method may comprise, when a hydration rank is derived which indicates that the clinical hydration status of the user is dehydration, comparing a derived hydration rank with stored hydration ranks and, based on this comparison, selecting a volume of rehydration fluid, and outputting the selected volume of rehydration fluid to the user.

When a hydration rank is derived which indicates that the clinical hydration status of the user is not dehydration, the computer-implemented method may not carry out these comparison and selection steps.

The rehydration fluid volume data table may associate volumes of rehydration fluid with stored other sensor information. The rehydration fluid volume data table may associate volumes of rehydration fluid with stored user input information. The rehydration fluid volume table may associate volumes of rehydration fluid with other factors such as a type of rehydration fluid.

The computer-implemented method may comprise, when a hydration rank is derived which indicates that the clinical hydration status of the user is dehydration, comparing acquired user input information with stored user input information and, based on this comparison, selecting a volume of rehydration fluid, and outputting the selected volume of rehydration fluid to the user. The computer-implemented method may comprise, when a hydration rank is derived which indicates that the clinical hydration status of the user is dehydration, comparing acquired other sensor information with stored other sensor information and, based on this comparison, selecting a volume of rehydration fluid, and outputting the selected volume of rehydration fluid to the user.

When a hydration rank is derived which indicates that the clinical hydration status of the user is not dehydration, the computer-implemented method may not carry out these comparison and selection steps.

In this way, a volume of rehydration fluid suitable for rehydrating the user, based on a derived hydration rank, which may indicate how dehydrated the user is, may be output to the user. Further, a volume of rehydration fluid suitable for rehydrating the user, based on data which may indicate a cause of dehydration of the user, may be output to the user.

The computer-implemented method may comprise storing a rehydration schedule data table. The rehydration schedule data table may associate a schedule by which rehydration fluid should be consumed with stored hydration ranks. The schedule may include a volume of rehydration fluid. The schedule may include a sub-volume of rehydration fluid and a time at which to drink the sub-volume of the rehydration fluid. The schedule may include a type of rehydration fluid.

The computer-implemented method may comprise, when a hydration rank is derived which indicates that the clinical hydration status of the user is dehydration, comparing a derived hydration rank with stored hydration ranks and, based on this comparison, selecting a schedule by which rehydration fluid should be consumed, and outputting the selected schedule to the user. When a hydration rank is derived which indicates that the clinical hydration status of the user is not dehydration, the computer-implemented method may not carry out these comparison and selection steps.

The rehydration schedule data table may associate a schedule by which rehydration fluid should be consumed with stored other sensor information. The rehydration schedule data table may associate a schedule by which rehydration fluid should be consumed with stored user input information.

The computer-implemented method may comprise, when a hydration rank is derived which indicates that the clinical hydration status of the user is dehydration, comparing acquired other sensor information with stored other sensor information and, based on this comparison, selecting a schedule by which rehydration fluid should be consumed, and outputting the selected schedule to the user. The computer-implemented method may comprise, when a hydration rank is derived which indicates that the clinical hydration status of the user is dehydration, comparing acquired user input information with stored user input information and, based on this comparison, selecting a schedule by which rehydration fluid should be consumed, and outputting the selected schedule to the user. When a hydration rank is derived which indicates that the clinical hydration status of the user is not dehydration, the computer-implemented method may not carry out these comparison and selection steps.

In this way, a rehydration fluid schedule suitable for rehydrating the user, based a hydration rank which may indicate how dehydrated the user is, may be output to the user. Further, a rehydration fluid schedule suitable for rehydrating the user, based on data which may indicate a cause of dehydration of the user or a type of dehydration of the user, may be output to the user.

If the derived hydration rank indicates that the clinical hydration status of the user is mild dehydration, and if the other sensor information and/or user input information indicates that the user has not undergone physical activity and that the user is not under thermal stress, the rehydration schedule may include a prompt to consume water. If the derived hydration rank indicates that the clinical hydration status of the user is mild dehydration, and if the other sensor information and/or user input information indicates that the user has undergone physical activity or that the user is under thermal stress, or is undergoing gastrointestinal problems, the rehydration schedule may include a prompt to consume an electrolyte fluid and one or more subsequent prompts to consume water.

If the derived hydration rank indicates that the clinical hydration status of the user is moderate dehydration, and if the other sensor information and/or user input information indicates that the user has not undergone physical activity and that the user is not under thermal stress and has not undergone gastrointestinal problems, the rehydration schedule may include a prompt to consume an electrolyte fluid and one or more subsequent prompts to consume water. If the derived hydration rank indicates that the clinical hydration status of the user is moderate dehydration, and if the other sensor information and/or user input information indicates that the user has undergone physical activity, is undergoing gastrointestinal problems, or that the user is under thermal stress, the rehydration schedule may include a prompt to consume an electrolyte fluid and one or more subsequent prompts to consume water.

Prompts to consume water may continue until euvolemia is reached.

The computer-implemented method may comprise re-deriving the hydration rank. The computer-implemented method may comprise re-selecting a rehydration schedule based upon the re-derived hydration index or hydration status.

The computer-implemented method may comprise storing a reassessment time data table. The reassessment time data table may associate stored reassessment times with stored hydration ranks.

The computer-implemented method may comprise, when a hydration rank is derived which indicates that the clinical hydration status of the user is dehydration, comparing the derived hydration rank with the stored hydration ranks and, based on this comparison, selecting a reassessment time. The computer-implemented method may comprise, after the reassessment time, re-acquiring data and re-deriving the hydration rank to obtain a reassessment hydration rank.

When a hydration rank is derived which indicates that the clinical hydration status of the user is not dehydration, the computer-implemented method may not carry out these comparison and selection steps.

The computer-implemented method may comprise, when the reassessment hydration index indicates that the user is dehydrated, alerting the user.

The computer-implemented method may comprise, when a hydration rank is derived which indicates that the clinical hydration status of the user is overhydration, comparing the derived hydration rank with the stored hydration ranks and, based on this comparison, selecting a reassessment time, and after the reassessment time, re-acquiring data and re-deriving the hydration rank to obtain a reassessment hydration rank.

When a hydration rank is derived which indicates that the clinical hydration status of the user is not overhydration, the computer-implemented method may not carry out these comparison and selection steps.

The computer-implemented method may comprise, when the reassessment hydration rank indicates that the user is overhydrated, alerting the user.

The alert may be output by the wearable device. The alert may be haptic, aural or visual. The alert may include a recommendation to drink. The alert may include a recommendation not to drink.

The reassessment time data table may associate stored reassessment times with other factors such as a type of rehydration fluid.

The computer-implemented method may comprise comparing a selected type of rehydration fluid selected from the rehydration fluid type data table with stored reassessment times and, based on this comparison, selecting a reassessment time.

The stored reassessment times may be clinically relevant. For example, they may be times which are clinically understood to be long enough for any action taken by the user to have had an impact on their hydration status.

In this way, the clinical hydration status of the user may be re-assessed after a time relevant to the initially derived hydration rank of the user and/or relevant to a recommended type of rehydration fluid. Further, there may be a physical output provided to the user when it is determined that the hydration status has not improved after the reassessment time.

The computer-implemented method may comprise receiving a reassessment time user input. The reassessment time user input may cause the computer-implemented method to re-derive the hydration rank of the user after a pre-determined reassessment time. The reassessment time user input may include a selection of a reassessment time by a user. The reassessment time user input may cause the computer-implemented method to re-derive the hydration rank of the user after a user-selected reassessment time.

The pre-determined reassessment time may be clinically relevant. For example, it may be a time which is clinically understood to be long enough for any action taken by the user to have had an impact on their hydration status.

The computer-implemented method may comprise, after an amount of time indicated by the reassessment time user input, re-acquiring data and re-deriving the hydration rank to obtain a reassessment hydration rank. The computer-implemented method may comprise, when the reassessment hydration rank indicates that the user remains dehydrated or overhydrated, alerting the user.

In this way, there may be a physical output provided to the user when it is determined that the hydration status of a user has not improved after a pre-determined period of time.

The computer-implemented method may comprise storing the hydration rank and storing the time at which the hydration rank is derived. The computer-implemented method may comprise obtaining time-correlated hydration rank information from previously derived stored hydration ranks and their corresponding stored times. The computer-implemented method may comprise outputting the time-correlated hydration rank information to the user such that the user can track how their hydration index varies over time.

In this way, an output may be provided which demonstrates the variation of the clinical hydration status of the user over time.

The computer-implemented method may comprise storing the other sensor information and/or user input information. The computer-implemented method may comprise obtaining time-correlated other sensor information and/or time correlated user input information from previously derived stored other sensor information and/or stored user input information and their corresponding stored times. The computer-implemented method may comprise outputting the time-correlated other sensor information and/or time-correlated user input information to the user such that the user can track how they vary over time.

The computer-implemented method may comprise using the time-correlated hydration rank information and the time-correlated other sensor information and/or user input information to correlate the hydration ranks with the other sensor information and/or user input information.

In this way, an output may be provided which demonstrates the impact of clinical factors on the clinical hydration status of the user.

In a seventh aspect, one or more embodiments of the invention provide a computer-implemented method for determining a hydration status of a user, the computer-implemented method comprising applying a model to bodily parameter data obtained from a user to obtain hydration information related to the user and deriving, from the hydration information, a hydration rank indicative of a hydration status of the user, wherein the hydration rank is a grade on a hydration rank scale.

The computer-implemented invention may include any one or any combination insofar as they are compatible of the features of the computer-implemented method according to the sixth aspect of the invention.

In a seventh aspect, one or more embodiments of the invention provide a computer program which when executed causes one or more processors to perform the method according to the sixth aspect or the seventh aspect of the invention.

The one or more processors may be components of the wearable device. The one or more processors may be components of a device external to the wearable device, for example a mobile device such as a mobile phone.

In an eighth aspect, embodiments of the invention provide a method for determining a hydration status of a user, the method comprising providing an optical sensing module on a wearable device worn by a user, providing a processor, and carrying out, by the processor, the computer-implemented method according to the sixth aspect of the invention, wherein the sensor is the optical sensing module on the wearable device.

In a ninth aspect, one or more embodiments of the invention provide a device comprising a processor, the processor configured to carry out the computer-implemented method according to the sixth aspect or the seventh aspect of the invention.

The device may comprise a storage medium storing the computer program according to any one or more embodiments of the present invention.

The device may be the wearable device.

The device may be a mobile device such as a mobile phone or tablet.

According to a tenth aspect, embodiments of the present invention provide a computer-implemented method for determining how well a user's body is regulating an analyte, the computer-implemented method comprising acquiring, from a sensor on a wearable device worn by the user, data including bodily parameter data related to the user, applying a model to the bodily parameter data to obtain analyte concentration information related to the user, wherein the model derives, from the analyte concentration information, a health score indicative of how well the user's body is regulating the analyte, wherein the health score is a given grade on a health score scale.

Thus, an indication of how well a user's body is regulating an analyte can be derived from wearable device-acquired data. In this way, a user can monitor how well their body is regulating the analyte during their normal routine. Therefore, the user may be provided with information which allows them to understand how their current lifestyle patterns are affecting their health. In this way, a user's overall health and wellbeing may be improved.

For example, a user may be assisted in losing weight, in understanding the effects of food and activity on their blood glucose levels, in optimizing their athletic performance and/or recovery, in reducing tiredness and/or in improving their overall health. Users may find being provided with a health score useful before bedtime, upon waking, before and after eating certain foods, in response to a certain diet, before, during and after exercise, or around activities of daily living.

Optional features of the computer-implemented method will now be described. The computer-implemented method may have any one, or any combination insofar as they are compatible, of the following features.

The health score may be an analyte concentration value. The analyte concentration value may be a concentration of an analyte in the blood. The analyte concentration value may be a clinical indication of an analyte concentration. The analyte concentration value may be an analyte concentration in units of mg/dL.

Thus, an analyte concentration value of a user can be derived from wearable device-acquired data. In this way, an analyte concentration value of the user may be provided during a user's normal routine.

The analyte may be a metabolic fuel. For example, the analyte may be glucose, ethanol, lactate, a ketone, or a fatty acid.

The computer-implemented method may be carried out on the wearable device, or on an external device. The external device may be a mobile device such as a mobile phone. The wearable device may be any device worn on the body, for example a watch, a ring, a necklace, bracelet, an ear bud, a skin contact patch, a glasses frame, or a strap worn around the wrist, the arm, the leg, or the torso.

The bodily parameter data may be an optical spectrum. The bodily parameter data may be a reflectance spectrum. The bodily parameter data may be a body tissue reflectance spectrum. The bodily parameter data may be a diffuse reflectance spectrum. The bodily parameter data may be an infrared diffuse reflectance spectrum.

In this way, an analyte concentration value of a user may be determined non-invasively. In other words, data for determining an analyte concentration value of a user may be obtained non-invasively.

The analyte concentration information may be indicative of the concentration of the analyte in the user's body.

The analyte concentration information may be a quantitative value associated with the bodily parameter data. The quantitative value associated with the bodily parameter data may be, for example, a position of a peak of the bodily parameter data, a wavelength shift of a peak of the bodily parameter data, a height of a peak of the bodily parameter data, or a width of a peak of the bodily parameter data.

The sensor may be an optical sensing module (i.e., an optical sensor) on the wearable device.

In this way, an analyte concentration value may be derived using an optical measurement. Further, an analyte concentration value may be provided non-invasively.

The optical sensing module may comprise a laser. The optical sensing module may comprise a plurality of lasers. Each laser of the plurality of lasers may operate at a wavelength that is different from the wavelength of the others. The optical sensing module may be configured to operate each laser one at a time. The optical sensing module may be configured to operate the plurality of lasers in a cycle according to a pre-determined schedule.

In this way, the optical sensing module may require fewer detectors. The optical sensing module may require only one detector. Advantageously then, the optical sensing module may be cheaper and simpler to manufacture than an optical sensing module which requires more detectors.

In one or more embodiments, the optical sensing module may have a sampling rate of 50,000 samples per second or fewer. In one or more embodiments, the sampling rate may be 1,000 samples per second or more. The data acquired from the optical sensing module may be an average of the samples. A laser on-time for the laser or for each of the plurality of lasers be 200 microseconds or more. As an example, the number of samples acquired in 200 microseconds may be 10. Two samples may be discarded in the processing. An example of a laser off-time for the laser or for each of the plurality of lasers may be 100 microseconds. As an example, the number of samples acquired in 100 microseconds may be 5. As an example, the time to perform 60 cycles may be 20 milliseconds. In one or more embodiments, the number of samples acquired in 20 milliseconds may be 1000 or more. A total measurement time may be 10 seconds or less. As an example, the number of cycles in 10 seconds may be 500. A plurality of total measurements may be taken. There may be an interval of 15 seconds between each total measurement.

The laser or the plurality of lasers may emit light in a wavelength band which is sensitive to changes in concentrations of the analyte for which the analyte concentration value is determined. The laser or the plurality of lasers may emit light in the wavelength band which covers wavelengths between at least 350 nm and no more than 2500 nm. The laser or the plurality of lasers may emit light in the visible wavelength band. The visible wavelength band may cover wavelengths roughly from 300 nm to 780 nm. The laser or the plurality of lasers may emit light in the infrared wavelength band. The laser or the plurality of lasers may emit light in the near-infrared wavelength band. The near infrared wavelength band may cover wavelengths roughly from 780 nm to 1000 nm. The laser or the plurality of lasers may emit light in the short wavelength infrared wavelength band. The short wavelength infrared wavelength band may cover wavelengths from roughly from 1000 nm to 2500 nm. The laser or a laser within the plurality of lasers may emit light within the wavelength range 2000 nm to 2500 nm. Within the wavelength range 2000 nm to 2500 nm, the spectral signatures for metabolic fuels such as glucose and lactate may be at their most distinct.

The optical sensing module may comprise one or more optical outputs for light originating from the laser or the plurality of lasers. Light from the laser or the plurality of lasers may exit the optical sensing module via one or more optical output ports. The optical sensing module may comprise a mirror to take the light from the plane of the optical sensing module and translate it into a direction more suitable for interrogating the surface. The direction may be orthogonal or substantially orthogonal to the plane of the optical sensing module.

The optical sensing module may include a transmitter photonic integrated circuit (PIC). The optical sensing module may comprise a substrate. The substrate may be a silicon substrate. The transmitter PIC may be located on the substrate. The transmitter PIC may include the laser or the plurality of lasers. The transmitter photonic integrated circuit (PIC) may be a silicon or silicon nitride photonic integrated circuit.

In use, light emitted from a laser of the optical sensing module may reflect or backscatter from a layer of the skin of the user. The optical sensing module may be configured to receive light backscattered from the skin of the user.

The optical sensing module may comprise a detector. The detector may be a photodetector. The detector may be located on the transmitter PIC such that the PIC is a transmitter/receiver PIC. The detector may be located separately from the transmitter PIC. The photodetector may be a silicon-based photodetector. The photodetector may be an InGaAs-based photodetector. The photodetector may be a germanium photodetector. The photodetector may be located on a receiver PIC that is vertically integrated and mounted on the same substrate as the transmitter PIC. The optical sensing module may comprise a plurality of detectors.

The optical sensing module may comprise an optical manipulation region. The optical manipulation region may comprise one or more of an optical modulator, an optical multiplexer, and additional optical manipulation elements.

The optical sensing module may be that disclosed in WO 2021/116766, the contents of which are incorporated herein by reference in its entirety.

In this way, the optical sensing module may have the ability to continuously take data. Therefore, the computer-implemented method may continuously receive data. Therefore, the analyte concentration value of the user may be continuously monitored. Further, the analyte concentration value of the user may be provided in real time.

The optical sensing module may comprise LEDs instead of lasers. LEDs may cheaper and simpler to manufacture than lasers. Lasers may allow for a more accurate indication of the analyte concentration value of the user.

The computer-implemented method may comprise acquiring, from the sensor on the wearable device worn by the user, a plurality of datasets, each of the plurality of datasets including bodily parameter data related to the user at a respective time. The computer-implemented method may further comprise applying the model to the bodily parameter data of each of the datasets to obtain a respective plurality of analyte concentration information related to the user, each of the plurality of analyte concentration information being indicative of a concentration of an analyte in the user's body, wherein the model derives, from the plurality of analyte concentration information, the health score.

In this way, an indication of how well a user's body is regulating an analyte may be derived. Thus, actionable data may be derived that can allow users to make choices that promote improvements in health and wellbeing.

The time at which each of the datasets in the plurality of datasets is detected by the senor may be selectable by the user. The time at which each of the datasets in the plurality of datasets is detected by the sensor may depend on the use case and/or utility of the computer-implemented method. The time intervals between which each of the datasets in the plurality of datasets is detected by the sensor on the wearable device may be selectable by the user. The time intervals between which each of the datasets in the plurality of datasets is detected by the sensor on the wearable device may depend on the use case and/or utility of the computer-implemented method. The total time over which the plurality of datasets is detected by the sensor on the wearable device may be selectable by the user. The total time over which the plurality of datasets are detected by the sensor on the wearable device may depend on the use case and/or utility of the computer-implemented method.

The use case may include whether the user is a healthy user or a user with an illness such as diabetes. For example, the use case may include that the user is non-diabetic or does not know if they are diabetic, that the user has Type I diabetes, that the user has Type II diabetes, or that the user has gestational diabetes. A use case of the computer-implemented method may be that the user is a healthy non-diabetic user, a health and fitness professional user, a public safety user, a pre-diabetic user, or a diabetic user. For a use case of a healthy user, only a low accuracy may be required.

The utility may include when the data is detected by the sensor. For example, the utility may include data being detected only when the user is in a fasting period (fasting utility), data being detected both when the user is in a fasting period and when the user is in a meal period (fasting and meal utility), or data being detected over a 24 hour period (24 hour utility).

A fasting period may be period of over 2 hours after a meal. A fasting period may be period of over 5 hours after a meal.

Fasting utility may avoid periods of high variability in analyte concentrations, as the data may be detected by the sensor in stable, consistent conditions such as during non-ambulatory sleep. Thus, only a low accuracy may be required. For fasting utility the time between which each of the datasets in the plurality of datasets is detected by the sensor on the wearable device may be 15 minutes. The total time over which the plurality of datasets is detected by the sensor on the wearable device may be 5 hours. The data may be smoothed over a 75 minute period.

For fasting and meal utility, the data detected in the fasting period may be detected when the user is sleeping. For fasting and meal utility, the data detected in the meal period may be a period of 1 hour, 75 minutes, or 2 hours after eating.

For 24 hour utility, data may be detected by the sensor periodically in each 24 hour period at regular intervals.

The use case and/or utility may be selectable by the user.

The computer-implemented method may continuously acquire data. The computer-implemented method may acquire data at pre-determined time points, for example the data may be every minute, every 5 minutes, every 10 minutes or every 15 minutes. The computer-implemented method may acquire data upon receiving a user input signal.

The computer-implemented method may derive the health score at predetermined time points, for example the health score may be derived after a period of time which is between 3 hours and 24 hours. For example, the health score may be derived every 24 hours. The computer-implemented method may derive the health score upon receiving a user input signal.

Typically, the bodily parameter data of each of the datasets are the same type of data. For example, typically the bodily parameter data of each of the datasets are optical spectra. The bodily parameter data of each of the datasets may be reflectance spectra. The bodily parameter data of each of the datasets may be body tissue reflectance spectra. The bodily parameter data of each of the datasets may be diffuse reflectance spectra. The bodily parameter data of each of the datasets may be infrared diffuse reflectance spectra.

The health score scale may run from 0 to 100. The health score scale may consist of health scores which are integers. The health score scale may consist of health scores which are multiples of 10. A lower health score may indicate a that the body is worse at regulating the analyte, A higher health score may indicate that the body is better at regulating the analyte.

The model deriving, from the plurality of analyte concentration information, the health score may include the model deriving, from the plurality of analyte concentration information, a respective plurality of analyte concentration values.

A scale of analyte concentration values may be sub-divided into a plurality of sub-ranges. The plurality of sub-ranges may be referred to as a plurality of analyte concentration zones or as a plurality of zones.

The computer-implemented method may comprise, for each derived analyte concentration value in the plurality of derived analyte concentration values, determining a sub-range of analyte concentration values which the derived analyte concentration value falls within. Deriving the health score may be deriving, using each of the determined sub-ranges, the health score.

Each of the analyte concentration zones in the plurality of analyte concentration zones may be a sub-range of analyte concentration values. Each analyte concentration zone in the plurality of analyte concentration zones may be indicative of a clinical analyte concentration status. An analyte concentration status may include a low analyte concentration, an ideal analyte concentration, or a high analyte concentration. A glucose concentration status may be hypoglycemia, ideal blood glucose or hyperglycemia.

Each of the analyte concentration zones in the plurality of analyte concentration zones may be a clinically defined analyte concentration zone. For example, for glucose as the analyte, the zones may be defined based on clinical thresholds established by the American Diabetes Association, the World Health Organization and/or American Association of Clinical Endocrinologists. An analyte concentration zone may be referred to as a zone.

The zones may be defined based on the use case and/or utility of the computer-implemented method.

The number of zones may vary depending on the use case and/or utility. The boundaries of the zones may vary depending on the use case and/or utility. That is, the sub-ranges of the analyte concentration values which define the zones may vary depending on the use case and/or utility. The analyte concentration status which each zone indicates may vary depending on the use case and/or utility. For example, for glucose as the analyte, the glucose concentration status which each zone indicates may vary depending on whether the user is diabetic, pre-diabetic or non-diabetic.

For example, for glucose as the analyte, there may be five zones. A fifth zone (zone 0) may be a sub-range of glucose concentration values of 54 mg/dL to <70 mg/dL. A first zone (zone 1) may be a sub-range of glucose concentration values of 70 mg/dL to <100. A second zone (zone 2) may be a sub-range of glucose concentration values of 100 mg/dL to <140 mg/dL. A third zone (zone 3) may be a sub-range of glucose concentration values of 140 mg/dL to <200 mg/dL. A fourth zone (zone 4) may be a sub-range of glucose concentration values of 200 mg/dL and above. These zones may be for the use case of non-diabetic users and/or prediabetic users. These zones may be for fasting and meal utility, or for 24 hour utility.

Thus, the limit set between Zones 3 and 4 may be 200 mg/dL for the use case of healthy, non-diabetic users and/or prediabetic users.

For example, for glucose as the analyte, there may be four zones. A fourth zone (zone 0) may be a sub-range of glucose concentration values of 54 mg/dL to <70 mg/dL. A first zone (zone 1) may be a sub-range of glucose concentration values of 70 mg/dL to <100. A second zone (zone 2) may be a sub-range of glucose concentration values of 100 mg/dL to <140 mg/dL. A third zone (zone 3) may be a sub-range of glucose concentration values of 140 mg/dL to <200 mg/dL. These zones may be for the use case of health and fitness professionals, public safety professionals and athletes. These zones may be for fasting and meal utility, or for 24 hour utility.

For glucose as the analyte, there may be 3 zones. A first zone (zone 1) may be a sub-range of glucose concentration values of 70 mg/dL to <100 mg/dL. A second zone (zone 2) may be a sub-range of glucose concentration values of 100 mg/dL to <140 mg/dL. A third zone (zone 3) may be a sub-range of glucose concentration values of 140 mg/dL to <200 mg/dL. These zones may be for the use case of non-diabetic users. These zones may be for fasting and meal utility, or for 24 hour utility.

For glucose as the analyte, there may be 2 zones. A first zone (zone 1) may be a sub-range of glucose concentration values of 70 mg/dL to <100 mg/dL. A second zone (zone 2) may be a sub-range of glucose concentration values of 100 mg/dL to <200 mg/dL. These zones may be for the use case of non-diabetic users. These zones may be for fasting utility.

In one or more embodiments, a fifth zone (Zone 0) may indicate hypoglycemia. A first zone (Zone 1) may indicate ideal resting blood glucose during sleep or during a fasting period. A fasting period may be period of over 5 hours after a meal. A second zone (Zone 2) may indicate ideal blood glucose within a 2 hour period after a meal. An ideal blood glucose peak in zone 2 may be less than 140 mg/dL. A third zone (Zone 3) may indicate hyperglycemia. A fourth zone (Zone 4) may indicate hyperglycemia. A user's glucose concentration value being within the fourth zone (zone 4) may indicate that the user is diabetic. These glucose concentration statuses may be for the use case of non-diabetic users.

In a 24 hour period, the ideal duration that a user's blood glucose levels are in the fifth zone (zone 0) may be less than 60 minutes. In a 24 hour period, the ideal duration that a user's blood glucose levels are less than 70 mg/dL may be less than 60 minutes, and the ideal duration that a user's blood glucose are less than 54 mg/dL may be less than 30 minutes or less than 15 minutes. In a 24 hour period, the ideal duration that a user's blood glucose levels are in the first zone (zone 1) may be more than 19 hours and 12 minutes or more than 21 hours and 36 minutes. In a 24 hour period, the ideal duration that a user's blood glucose levels are in the second zone (zone 2) may be less than 4 hours and 48 minutes or less than 2 hours and 24 minutes. In a 24 hour period, the ideal duration that a user's blood glucose levels are in the third zone (zone 3) may be less than 60 minutes or less than 30 minutes. In a 24 hour period, the ideal duration that a user's blood glucose levels are in the fourth zone (zone 4) may be 0 minutes.

The ideal percentage of time that a user's blood glucose levels are in the fifth zone (zone 0) may be less than 4%. The ideal percentage of time that a user's blood glucose levels are less than 70 mg/dL may be less than 4%. The ideal percentage of time that a user's blood glucose levels are less than 54 mg/dL may be less than 2% or less than 1%. The ideal percentage of time that a user's blood glucose levels are in the first zone (zone 1) may be more than 80% or more than 90%. The ideal percentage of time that a user's blood glucose levels are in the second zone (zone 2) may be less than 20% or less than 10%. The ideal percentage of time that a user's blood glucose levels are in the first or second zones (zone 1 or zone 2) may be over 95%. The percentage of time that a user's blood glucose levels are typically between 70 mg/dL and 140 mg/dL may be between 91% and 99.2%. The ideal percentage of time that a user's blood glucose levels are in the third zone (zone 3) may be less than 4% or less than 2%. The ideal percentage of time that a user's blood glucose levels are over 140 mg/dL may be between 0% and 4.4%. The ideal percentage of time that a user's blood glucose levels are in zone 4 may be 0%.

For glucose, there may be 6 zones. A seventh zone (zone −2) may be a sub-range of glucose concentration values of <54 mg/dL. A sixth zone (zone −1) may be a sub-range of glucose concentration values of 54 mg/dL to <70 mg/dL. A first zone (zone 1) may be a sub-range of glucose concentration values of 70 mg/dL to <130 mg/dL. A second zone (zone 2) may be a sub-range of glucose concentration values of 130 mg/dL to <180 mg/dL. A third zone (zone 3) may be a sub-range of glucose concentration values of 180 mg/dL to <250 mg/dL. A fourth zone (zone 4) may be a subrange of glucose concentration values of 250 mg/dL to <600 mg/dL. These zones may be for the use case of diabetic users.

Thus, an example of the limit set between the second and third zones (zones 2 and 3) may be 180 mg/dL.

A seventh zone (Zone −2) may indicate level 2 and level 3 hypoglycemia. A sixth zone (Zone −1) may indicate level 1 hypoglycemia. The first and second zones (Zones 1 and 2) may indicate an ideal blood glucose. The third zone (Zone 3) may indicate level 1 hyperglycemia. The fourth zone (Zone 4) may indicate level 2 hyperglycemia. These glucose concentration statuses may be for the use case of diabetic users.

The computer-implemented method may comprise acquiring a plurality of time stamps indicative of the respective plurality of times at which the plurality of datasets are acquired. Deriving the health score may be deriving, using each of the determined sub-ranges and the plurality of time stamps, the health score of the user.

Each of the time stamps be acquired from the wearable device. The computer-implemented method may further comprise generating each of the stamps.

Deriving the health score may comprise calculating, using the plurality of sub-ranges and the plurality of time stamps, a duration of time in which a user's analyte concentration remains within a given sub-range. Deriving the health score may further comprise comparing the duration of time in which the user's analyte concentration remains within a given sub-range with a target time in the given sub-range. For example, if a user's analyte concentration remains within a sub-range (or zone) which is indicative of high analyte concentration for a long time, a health score may be derived which indicates that a user's body is sub-optimally regulating the analyte. If a user's analyte concentration remains within a sub-range (or zone) which is indicative of an ideal analyte concentration for a long time, a health score may be derived which indicates that a user's body is optimally regulating the analyte.

In this way, a health score may be derived which is correlated with ideal analyte regulation by the body. Thus, a health score may be derived which indicates how well a user's body is regulating an analyte.

A longer duration of time that a user's glucose concentration remains within a first zone (zone 1) may indicate better glycemic control. The duration of time that a user's glucose concentration remains within a second zone (zone 2) may indicate how well a user's glucose concentration levels recover after eating. The duration of time that a user's glucose concentration remains within the second zone (zone 2) may indicate how well a user's body utilizes blood glucose. A longer duration of time that a user's glucose concentration remains within the second zone (zone 2) during a fasting period may indicate poor glycemic control and may indicate pre-diabetes. A fasting period may be a period of time which is over two hours after a meal. A longer duration of time that a user's glucose concentration remains within the third or fourth zones (zone 3 or zone 4) may indicate worse glycemic control and may indicate pre-diabetes or diabetes. Any duration of time that a user's glucose concentration remains within the fourth zone (zone 4) may indicate poor glycemic control.

The model deriving the health score may comprise calculating, using the plurality of analyte concentration values a variability of the plurality of analyte concentration values. The model deriving the health score may comprise calculating, using the plurality of analyte concentration values, a mean analyte concentration value. The variability may be assessed relative to the mean of the plurality of analyte concentration values. The variability may be a standard deviation, a coefficient of variation, a mean amplitude glucose excursion, a J-index, a mean absolute difference, or a mean absolute glucose.

A higher variability may indicate that the user's body is not regulating the analyte as well. A higher variability may result in a lower health score. A lower variability may indicate that the user's body is regulating the analyte better. A lower variability may result in a higher health score.

In this way, a health score may be derived which is correlated with ideal analyte regulation by the body. Thus, a health score may be derived which indicates how well a user's body is regulating an analyte.

The model deriving the health score may comprise a step of calculating a number of zone points and a step of calculating a number of variability points. The health score may be the sum of the zone points and the variability points.

Calculating the zone points may comprise determining if, for the total time over which the plurality of datasets are detected by the sensor, the time in a first zone (zone 1) is greater than 90% of the total time. Calculating the zone points may comprise setting the number of zone points to (75+the time in zone 1−90) if the time in the first zone (zone 1) is greater than 90% of the total time. Calculating the zone points may comprise setting the number of zone points to (75−the time in zone 1−90) if the time in the first zone (zone 1) is not greater than 90% of the total time.

Calculating the number of variability points may comprise calculating the coefficient of variance (CV) of the plurality of analyte concentration values. Calculating the number of variability points may comprise determining if the CV is less than e.g. 17. In such an example, calculating the number of variability points may comprise setting the number of variability points to 15 if it is determined that the CV is less than 17. Calculating the number of variability points may comprise setting the number of variability points to—(CV−17) if it is determined that the CV is not less than 17.

The model deriving the health score may comprise calculating, using the plurality of analyte concentration values, an average analyte concentration value. The health score may be a time-averaged analyte concentration value. The health score may be the mean of the plurality of analyte concentration values.

In this way, a more accurate health score may be determined as compared to a health score which is a single derived analyte concentration value.

The health score may be derived retrospectively using a plurality of datasets detected by the sensor over a given period of time. For example, a health score may be derived at the end of the day using a plurality datasets detected by the sensor that day.

The plurality of analyte concentration values may be metabolic fuel concentration values. The health score may be indicative of how well the user's body is regulating a metabolic fuel. For example, the plurality of analyte concentration values may be glucose concentration values, ethanol concentration values, lactate concentration values, ketone concentration values, or fatty acid concentration values. The health score may be indicative of how well the user's body is regulating glucose, ethanol, lactate, a ketone or a fatty acid.

The model may include a machine learning model. The model may include a regression model. The model may include a partial least squares (PLS) regression model. The model may include a principal component analysis (PCA) model. The PCA model may be applied before the regression model.

Deriving, from the plurality of analyte concentration information, the health score may be carried out by a machine learning model. Deriving, from the plurality of analyte concentration information, the health score may be carried out by a PLS regression model, and/or a PCA model.

Deriving, from the plurality of analyte concentration information, a respective plurality of analyte concentration values may be carried out by a machine learning model. Deriving, from the plurality of analyte concentration information, a respective plurality of analyte concentration values may be carried out by a PLS regression model, and/or a PCA model.

The model may have been trained using training data. The training data may include a plurality of training datasets, each of the training datasets comprising bodily parameter data, and each of the training datasets acquired from the wearable device. The training data may include clinical labels. Each training dataset may be associated with a respective one of the clinical labels. Each of the clinical labels may include an output of a standard clinical analyte concentration test. The output of the standard clinical analyte concentration test may be a clinical measurement of an analyte concentration value. For example, for glucose as the analyte, the output of the standard clinical glucose concentration test may be a clinical measurement of glucose concentration. The clinical data which is used to derive the output of the standard clinical analyte concentration test may have been acquired at the same time as or at a similar time to the acquisition of the corresponding training dataset, for example within the same 5 minute interval, 15 minute interval, or 1 hour interval.

The model trained or generated in this way is not limited to a machine learning model.

The model may include an offline model. The offline model may have been trained using batch training data.

The offline model may derive the health score.

The training data may have been acquired from a single subject. The single subject may be the user of the wearable device. The training data may have been acquired from a plurality of subjects.

The model may comprise one or more pre-processing steps. The one or more pre-processing steps may comprise applying a statistical model to the data acquired from the sensor to validate the data acquired from the sensor.

In one or more embodiments, the computer-implemented method may comprise a step of detecting whether the bodily parameter data has been acquired from a human body.

Validating the data may comprise determining whether the data has been acquired from a human user. In addition, or alternatively, validating the data may comprise determining whether the data is anomalous data.

In this way, the effect of any anomalous data, or any incorrectly acquired data on the accuracy of the hydration status indication is reduced.

The statistical model may be generated using a plurality of training datasets, each dataset comprising bodily parameter data. The plurality of training datasets may have been acquired from a plurality of subjects. To validate the data acquired from the sensor, the statistical model may determine whether the data acquired from the sensor falls within a pre-determined number of standard deviations, for example within 2 standard deviations, of the plurality of training datasets. To validate the data acquired from the sensor, the statistical model may calculate a Mahalonobis distance metric between the data acquired form the sensor and the plurality of training datasets, and determine whether the Mahalonobis distance metric is within a given threshold.

The one or more pre-processing steps may comprise applying a baseline correction to the data. Applying the baseline correction to the data may comprise subtracting baseline data from the acquired data. The baseline data may be derived from data previously acquired from the user. The baseline data may be average data acquired from the user. The average data may have been derived from data acquired from the user over a long time period, for example, over 24 hours, 1 week, or 1 month.

In this way, the effect of noise in the data may be reduced. In this way, the indication of how well the user's body is regulating the analyte may be more accurate.

The one or more pre-processing steps may comprise assessing the quality of the data. If in assessing the quality of the data it is determined that the quality of the data falls below a pre-determined threshold, the data may be discarded.

The model may include one or more-post processing steps.

The one or more post-processing steps may include smoothing the time profile of the plurality of derived analyte concentration values.

The one or more post-processing steps may include truncating the time profile of the plurality of derived analyte concentration values to a time range. The time range may be selectable by the user. The time range may be a pre-determined time range.

In this way, a health score may be obtained for a desired time range.

The computer-implemented method may comprise acquiring other sensor information in addition to the analyte concentration information. The computer-implemented method may comprise acquiring user input information.

The other sensor information may be acquired from the sensor on the wearable device. The other sensor information may be acquired from an additional sensor. The additional sensor may be external to the wearable device. The additional sensor may be on the wearable device. The other sensor information may be obtained from other bodily parameter data related to the user. The other bodily parameter data may be an optical spectrum.

The other sensor information may include clinically relevant information. The other sensor information may include one or more of body temperature information obtained from a temperature sensor, heart rate information obtained from a heart rate sensor, blood oxygen saturation information obtained from a blood oxygen saturation sensor, respiratory rate information obtained from a respiratory rate sensor, hydration information obtained from a hydration sensor, accelerometer and motion information obtained from an accelerometer or a motion sensor, heart rate variability information obtained from a heart rate sensor, alcohol concentration, sleep/wake information obtained from a sleep sensor, and blood pressure information obtained from a blood pressure sensor.

The heart rate sensor, the blood oxygen saturation sensor and the respiration rate sensor may be a PPG sensor. The blood pressure sensor and the heart rate sensor may be an SPG sensor. The temperature sensor may be a short-wavelength infrared sensor. The heart rate information and the heart rate variability information may be obtained from an electrocardiogram.

The other sensor information may be or may be measured using physiological indicators. Physiological indicators may include tissue perfusion or ischemia, infection, decompensation, pain, performance, overtraining, movement/activity, core body temperature, resting heart rate, real-time heart rate, maximum heart rate, heart rate thresholds, VO₂ or VO₂ maximum, intensity, sleep quality, sleep disturbance, apnea hypopnea index (AHI), oxygen desaturation index (ODI), metabolic equivalent of tasks (METs), metabolic health, caloric cost, or general health status.

For example, the blood oxygen saturation information may be an oxygen desaturation index. The heart rate information may be a VO₂ measurement or a maximum VO₂ measurement. The heart rate information may be an MET measurement. The respiration rate information may be an apnea-hypopnea index or a respiratory disturbance index.

The user input information may be acquired from a user input into the wearable device. The user input information may be acquired from a user input into an external device which may be a mobile device such as a mobile phone. The user input information may include one or more of weight information, height information, activity information, diet information, fluid intake information, sodium intake information, illness information, intoxication information, blood pressure information, sleep duration information, sleep quality information, gender information, age information and heart rate information.

The user input information may include a value on a clinically relevant scale. For example, weight information may include a mass in kg, or pounds. Weight information may include a body mass index (BMI). Height information may include a height in cm or inches. Activity information may include an amount of calories burnt. Activity information may include information from which an amount of calories burnt could be calculated, for example a type of exercise and a duration of the exercise. Activity information may also include duration of an activity completed, type of activity completed or other information related to the user's experience of the activity, such as perceived exertion. Diet information may include a food group (such as fat, carbohydrate or protein) consumed. Diet information may include an amount of calories consumed. Diet information may include information from which an amount of calories consumed could be calculated, for example a type of food and an amount of food. Fluid intake information may include a volume of fluid consumed. Fluid intake information may include a type of fluid consumed, for example water or an electrolyte fluid. Sodium intake information may include a mass of sodium in mg. Sodium intake information may include information from which a mass of sodium could be calculated, for example a type of food and an amount of food. Illness information may include a temperature. Illness information may include a type of diagnosed illness, a duration of illness or other information related to symptoms. Intoxication information may include a number of alcohol units consumed. Intoxication information may include information from which a number of alcohol units consumed could be calculated, for example a type of alcohol and a volume consumed. Intoxication information may include a number of days in which alcohol has been consumed. Blood pressure information may include a measurement in mmHg. Blood pressure information may include a ratio of systolic pressure to diastolic pressure, where each pressure may be a measurement in mmHg. Sleep duration may include a time in hours and/or minutes. Sleep quality information may include a total sleep time, and/or a total wake time in hours and/or minutes. Gender information may include whether the sex of the user is male or female. Age information may include an age in years. Heart rate information may include a maximum heart rate or a VO₂ measurement.

The computer-implemented method may comprise storing a recommendation data table.

The recommendation data table may associate recommendations with stored health scores. The computer-implemented method may comprise comparing a derived score with the stored health scores and, based on this comparison, selecting a recommendation to output to the user. The recommendation may be output to the user.

The parameter values may be health scores, durations of time in which a user's analyte concentration remains within a given sub-range, rates at which a user's analyte concentration changes, analyte concentration values, or peak analyte concentration values of a plurality of analyte concentration values.

The recommendations may be actions for the user to take, for example “conduct physical activity”, “assess dietary choices”, or “consult a clinician”.

The selected recommendation may be one which is clinically understood to improve the health score of the user. For example, for glucose as the analyte, the recommendations may aim to increase the time that the user's blood glucose levels spend within a first zone (zone 1).

In this way, a recommendation appropriate to improving how well a user's body regulates an analyte may be selected. For example, it has been well established that both acute bouts of exercise and chronic training have positive effects on both insulin sensitivity and blood glucose regulation. In addition, healthy nutritional habits preclude the deleterious effects of developing insulin resistance and glucose intolerance.

If the derived parameter indicates that a user's blood glucose levels have been in a fourth zone (Zone 4), the selected recommendation may include a prompt to visit a clinician.

If the derived parameter indicates that a user's blood glucose levels have been in the third zone (Zone 3), or have been in the third zone for over a predetermined threshold duration, the selected recommendation may include a prompt to conduct physical activity and/or to assess the food that they have recently consumed. If the derived parameter indicates that a user's blood glucose levels have risen into the third zone at a rate over a predetermined threshold rate, the selected recommendation may include a prompt to conduct physical activity and/or to assess the food that they have recently consumed. If the derived parameter indicates that a user's blood glucose levels have been in the third zone (Zone 3), or have been in the third zone (Zone 3) for over a predetermined threshold duration, the selected recommendation may include a prompt to consult a clinician. If the derived parameter indicates that a user's blood glucose levels have been in the third zone (Zone 3) during a fasting period, the selected recommendation may include a prompt to consult a clinician. If the derived parameter indicates that a user's blood glucose levels have frequent postprandial peaks in the third zone (Zone 3), and/or that a user's blood glucose levels are over a pre-determined threshold of variability, the selected recommendation may be an alert.

If the derived parameter indicates that a user's blood glucose levels have been in the second zone (Zone 2) for over a predetermined threshold duration, the selected recommendation may include a prompt to conduct physical activity and/or to assess their dietary choices. If the derived parameter indicates that a user's blood glucose levels have been in the second zone (Zone 2) for over a predetermined threshold duration, the selected recommendation may include a prompt to consult a clinician. If the parameter indicates that a user's blood glucose levels have been in the second zone (Zone 2) during a fasting period, the selected recommendation may include a prompt to consult a clinician.

If the derived parameter indicates that a user's blood glucose levels have been in the first zone (Zone 1) for over a predetermined threshold duration, the selected recommendation may include a prompt to the user to continue their current behavior. If the derived parameter indicates that a user's blood glucose levels have been in the first zone (Zone 1) for under a predetermined threshold duration, the selected recommendation may include a prompt to conduct physical activity and/or to assess the food that they have recently consumed.

If the derived parameter indicates that a user's blood glucose levels have been in the fifth zone (Zone 0) for over 15 minutes, the selected recommendation may include a prompt to consume a carbohydrate supplement and/for to decrease physical activity. If the derived parameter indicates that a user's blood glucose levels have been in the fifth zone (Zone 0) for over a pre-determined threshold duration, the selected recommendation may include a prompt to consult a clinician.

If the derived parameter indicates that a user's blood glucose levels have been in the sixth zone (Zone −1 for over 15 minutes, the selected recommendation may include a prompt to consume a carbohydrate supplement and/for to decrease physical activity. If the derived parameter indicates that a user's blood glucose levels have been in the sixth zone (Zone −1) during a fasting period, the selected recommendation may include a prompt to consult a clinician. If the derived parameter indicates that a user's blood glucose levels have been in the sixth zone (Zone −1) for over a threshold duration, the selected recommendation may include a prompt to consult a clinician.

If the derived parameter indicates that a user's blood glucose levels have been in the seventh zone (Zone −2) for over 15 minutes, the selected recommendation may include a prompt to consume a carbohydrate supplement and/or to consult a clinician. If the derived parameter indicates that a user's blood glucose levels have been in the seventh zone (Zone −2) for two or more 15 minute periods, the selected recommendation may include a prompt to consult a clinician.

The recommendation data table may associate recommendations with stored other sensor information. The recommendation data table may associate recommendations with stored user input information. The computer-implemented method may comprise comparing acquired other sensor information with stored other sensor information. Selecting the recommendation to output to the user may be further based upon this comparison. The computer-implemented method may comprise comparing acquired user input information with stored user input information. Selecting the recommendation to output to the user may be further based upon this comparison.

In this way, the recommendation provided to the user may be more effective at improving how well the user's body regulates the analyte, by taking into account other clinically relevant information.

For example, if the derived parameter indicates that the recommendation should be a prompt to conduct physical activity or to assess dietary choices, and the user input information and/or other sensor information indicates that the user is conducting physically activity, the selected recommendation may be a prompt to assess dietary choices. If the derived parameter indicates that the recommendation should be a prompt to conduct physical activity or to assess dietary choices, and the user input information and/or other sensor information indicates that the user is not conducting physically activity, the selected recommendation may be a prompt to conduct physical activity.

Further, for example, a user input that a user has in taken a carbohydrate supplement may provide a reason for the derived parameter indicating a high blood glucose level and may thus affect the selected recommendation.

The recommendations may be output at a pre-determined frequency. The frequency may depend upon the value of the derived parameter. For example, the frequency may depend upon rate of change of the analyte concentration value and/or on a peak analyte concentration value. The frequency may depend upon the other sensor information and/or the user input information. For example, dietary user input information and/or accelerometer data may affect the frequency at which prompts for the user to conduct physical activity or to assess their food choices are output.

The computer-implemented method may comprise applying a user-recommendation machine learning model to the health score. The user-recommendation machine learning model may take as an input the health score. The user-recommendation machine learning model may output a recommendation based on the health score.

The user-recommendation machine learning model may have been trained using training data. The training data may comprise a plurality of health scores and a respective plurality of context labels. Each of the context labels may comprise user input information and/or other sensor information corresponding to the respective health score.

In this way, health scores of a user can be correlated with clinical data which indicates characteristics and/or behaviors of the user. Thus, recommendations can be derived which may improve the health score of the user by changing the characteristics and/or behaviors of the user.

The training data may have been acquired from a single subject. The single subject may be the user of the wearable device. The training data may have been acquired from a plurality of subjects.

The computer-implemented method may comprise applying the user-recommendation machine learning model to the health score and to the user input information and/or the other sensor information. The user-recommendation machine learning model may take as inputs the health score and the user input information and/or the other sensor information. The user-recommendation machine learning model may output a recommendation based on the health score and the user input information and/or the other sensor information.

The computer-implemented method may comprise storing the health score and storing the time at which the health score is derived. The computer-implemented method may comprise obtaining, from previously derived stored health scores and their corresponding stored times, time-correlated health score information. The computer-implemented method may further comprise outputting the time-correlated health score information to the user such that the user can track how their health score varies over time.

The computer-implemented method may comprise storing the derived analyte concentration value and storing the time at which the analyte concentration value is derived. The computer-implemented method may comprise obtaining, from previously derived stored analyte concentration values and their corresponding stored times, time-correlated analyte concentration value information.

The computer-implemented method may comprise storing the other sensor information and/or user input information. The computer-implemented method may comprise obtaining time-correlated other sensor information and/or time correlated user input information from previously derived stored other sensor information and/or stored user input information and their corresponding stored times.

The computer-implemented method may comprise using the time-correlated health score rank information and the time-correlated other sensor information and/or user input information to correlate the health scores with the other sensor information and/or user input information.

In this way, health scores of a user can be correlated with clinical data which indicates characteristics and/or behaviors of the user. Thus, the impact of clinical factors on the health score of the user may be demonstrated. For example, spikes in blood glucose may be correlated with nutritional choices and/or activity levels.

Therefore, the user may be provided with information which allows them to understand how their lifestyle patterns are affecting their health.

The computer-implemented method may comprise outputting an output to the user. The output may be output to the user in real time. The output may be output to the user at predetermined time points, for example the output may be output to the user, for example, every hour, every 6 hours, every 12 hours or every 24 hours. The output may be output to the user upon receiving a user input signal. Outputting the output to the user may mean displaying the output on the wearable device. Outputting the output to the user may mean displaying the output on an external device, which may be a mobile device such as a mobile phone. Outputting the output to the user may use an application on an external device.

The output may include the health score, the analyte concentration, or the mean analyte concentration value. The output may include a duration that the user's analyte concentration values spent in each zone over a given period. The given period may, for example, be 24 hours.

In this way, information about how well the user's body is regulating an analyte may be provided to the user in a more accessible way than can be provided by laboratory tests. Users and their clinicians can make choices about the user's health.

The output may include the selected recommendation.

In this way a recommendation appropriate to improving how well a user's body regulates an analyte may be output to the user.

The output may include the time-correlated health score information. The output may include the time-correlated analyte concentration value information. The output may include the time-correlated other sensor information and/or the time correlated user input information. The output may include a target health score line.

In this way, the user can track how their health score varies over time.

The computer-implemented method may comprise outputting the time-correlated other sensor information and/or time-correlated user input information to the user such that the user can track how they vary over time.

For fasting utility, the output may include the previous day's average analyte concentration value presented as a health score. The output may further include time-correlated health score information.

For fasting and meal utility, the output may include one or more of the previous day's average analyte concentration around meals. For example, if the user had 2 meals in the previous day, the output may include 2 average analyte concentrations. The average analyte concentrations may be presented as the health score.

For 24-hour utility, or otherwise, the output may include the health score. The output may further include the durations that the user's analyte concentration values spent in each zone in the previous 24-hour period.

In an eleventh aspect, one or more embodiments of the invention provide a computer-implemented method for determining how well a user's body is regulating an analyte, the computer-implemented method comprising applying a model to a plurality of bodily parameter data acquired from a user to obtain a respective plurality of analyte concentration information related to the user, each of the plurality of analyte concentration information being indicative of a concentration of an analyte in the user's body, wherein the model derives, from the plurality of analyte concentration information, the health score.

The computer-implemented invention may include any one or any combination insofar as they are compatible of the features of the computer-implemented method according to the ninth aspect of the invention.

In a twelfth aspect, one or more embodiments of the invention provide a computer program which when executed causes one or more processors to perform the method according to the tenth aspect or the eleventh aspect of the invention.

The one or more processors may be components of the wearable device. The one or more processors may be components of a device external to the wearable device, for example a mobile device such as a mobile phone.

In a thirteenth aspect, one or more embodiments of the invention provide a method for determining an analyte concentration value of a user of a user comprising providing an optical sensing module on a wearable device worn by a user, and providing a processor, and carrying out, by the processor, the computer-implemented method according to the tenth aspect of the invention, wherein the sensor is the optical sensing module.

In a fourteenth aspect, one or more embodiments of the invention provide a device comprising a processor, the processor configured to carry out the computer implemented method according to the tenth aspect or the eleventh aspect of the invention.

The device may comprise a storage medium storing the computer program according to any one or more embodiments of the present invention

The device may be the wearable device.

The device may comprise the optical sensing module according to any one or more embodiments of the present invention.

The device may be a mobile device such as a mobile phone or tablet.

In an aspect, one or more one or more embodiments of the invention provide a device comprising a processor, the processor configured to carry out a plurality of computer implemented methods, the plurality of computer implemented methods being any combination of the computer-implemented methods according to aspects of the invention.

The device may comprise a storage medium storing the computer program according to any one or more embodiments of the present invention

The device may be the wearable device.

The device may comprise one or more optical sensing modules, the one or more optical sensing modules each being according to any one or more embodiments of the present invention. The one or more optical sensing modules may be configured to detect data for the plurality of computer-implemented methods.

According to an aspect of the invention, there is provided a computer-implemented method for determining a notification to output to a user, the method comprising storing a notification data table, the notification table associating notifications with a plurality of stored physiological ranks, comparing a plurality of derived physiological ranks with the stored physiological ranks and, based on this comparison, selecting a notification to output to the user; and, outputting the selected notification to the user, wherein the plurality of derived physiological ranks include at least two of a physiological rank derived in accordance with one or more embodiments of the present invention, a hydration rank derived in accordance with one or more embodiments of the invention, and a health score derived in accordance with one or more embodiments of the present invention.

For example, the computer-implemented method may comprise deriving a temperature, a hydration rank and a health score.

The notification data table may be a table according to one or more embodiments of the invention. For example, the notification data table may be the recommendation data table.

The computer-implemented method may be carried out by a machine learning algorithm.

According to an aspect of the invention, there is provided a computer-implemented method for deriving an overall rank indicative of the health of a user, the computer-implemented method comprising deriving a plurality of physiological ranks, and combining the derived plurality of physiological ranks to obtain an overall rank, wherein the plurality of physiological ranks include at least two of a physiological rank derived in accordance with one or more embodiments of the invention, a hydration rank derived in accordance with one or more embodiments of the present invention, and a health score derived in accordance with the invention.

For example, a derived temperature and a derived hydration rank may be combined to obtain an overall rank. The derived plurality of physiological ranks may be output. The overall rank may be output.

A computer-implemented method according to aspects of the invention may be beneficial in multiple areas.

For example, ranks relevant to the cardiac health of the user may be derived. For example, a heart rate rank, a heart rate variability rank, a respiratory rate rank, an oxygen saturation rank, and/or a blood pressure rank may be derived. The sensor data may be ECG data or calculations for VO₂, VO₂ max and METs. The other sensor information may be ECG data or calculations for VO₂, VO₂ max and METs. The individual ranks may be derived and output, as well as an overall rank which combines one or more of the ranks. The output ranks may indicate responses to physical, environmental and mental stressors, medical intervention and medication. Notifications output may include mindful breathing, medication reminders, dietary and activity prompts, stress and sleep assessments, and lifestyle habits logging. This application of the computer-implemented method may be used by users wishing to monitor their general health; users with perceived stress; athletes during training, performance and recovery; vulnerable users; users with cardiovascular disease; users with special diets; users with pulmonary conditions; uses with infections; users with sleep apnea; users in a clinical scenario.

Ranks relevant to athletic training and recovery may be derived. For example, a hydration rank, a temperature and/or a health score may be derived. The sensor data may be ECG data or calculations for VO₂, VO₂ max and METs. The other sensor information may be ECG data or calculations for VO₂, VO₂ max and METs. The individual ranks may be used to optimize performance, optimize training programs and optimize acclimatization. An overall rank from combining the individual ranks may identify recovery and training preparedness, overtraining and recovery from illness. The overall rank may be based on data acquired post-activity. This application of the computer-implemented method may be used by users who are professional athletes, endurance athletes, or recreationally active users.

Ranks relevant to the mental health of the user may be derived. A hydration rank, a temperature and/or a health rank may be derived. Individual ranks/an overall rank may be derived which indicate responses to and recovery from perceived stress. Notifications output may include mindful breathing, dietary and activity prompts, stress and lifestyle habits assessments. This application of the computer-implemented method may be used by users who are part of the general population, who have occupations in health and safety, who are travelling, who are assessing their athletic performance, and who are undergoing therapy or counselling.

Ranks relevant to the sleep quality of the user may be derived. A hydration rank, a temperature rank, a health rank, an AHI, an RDI and/or an ODI may be derived, as well as an overall rank which combines one or more of the ranks. The individual ranks may indicate diurnal metrics while asleep. This application of the computer-implemented method may be used by users who are part of the general population, athletes, those with occupations in health and safety, those travelling, those undergoing acclimatization, those undergoing therapy or counselling, those with anxiety or depression, those with insomnia, and those with sleep related breathing disorders.

Ranks relevant to fertility awareness, pregnancy and menopause may be derived. A hydration rank, a temperature rank and/or a health score may be derived. Trending information related to menstrual cycle tracking and ovulation prediction may be derived. Temperature changes during activity and over time may be derived. Changes in core body temperature may be derived. User input information and other sensor information may be related to follicular and luteal phase hormones. This application of the computer-implemented method may be used by users who wish to carry out natural family planning, who have abnormal cycles and ovulation patterns, who are pregnant and wish to carry out physical activity, and who experience menopausal hot flushes.

### BRIEF DESCRIPTION OF THE DRAWINGS

These and other features and advantages of the present invention will be appreciated and understood with reference to the specification, claims, and appended drawings wherein:

FIG. 1 shows a flowchart of example steps of the computer-implemented method;

FIG. 2 is a schematic diagram of an optical sensing module that may be configured to carry out the computer-implemented method;

FIG. 3 shows a flowchart of example steps of the computer-implemented method;

FIG. 4 is a depiction of a further example of steps of a computer-implemented method;

FIG. 5 depicts an example of a temperature scale;

FIG. 6 shows a plot of temperature against time, and shows time-correlated user-input information;

FIG. 7A is an example of an output of the computer-implemented invention in the form of a graphical user interface (GUI), for example on a mobile device;

FIG. 7B is an example of an output of the computer-implemented invention in the form of a graphical user interface (GUI), for example on a mobile device;

FIG. 8 shows a further flowchart of example steps of the computer-implemented method;

FIG. 9 depicts an example of hydration information in the form of a hydration rank scale;

FIG. 10 is a depiction of a further example of steps of a computer-implemented method;

FIG. 11 is an example of an output of the computer-implemented invention in the form of a graphical user interface (GUI), for example on a mobile device;

FIG. 12A depicts a further example of hydration information in the form of a hydration rank scale; and

FIG. 12B depicts a further example of hydration information in the form of a hydration rank scale.

FIG. 13 shows a flowchart of example steps of the computer-implemented method;

FIG. 14 is a depiction of a further example of steps of a computer-implemented method;

FIG. 15 is a depiction of a further example of steps of a computer-implemented method;

FIG. 16 shows plots of blood glucose levels against time for a non-diabetic person and for a diabetic person;

FIG. 17A is a depiction of a range of blood glucose levels and of zones defined by sub-ranges of the blood glucose levels for the use case of a healthy user;

FIG. 17B is a depiction of a range of blood glucose levels and of zones defined by sub-ranges of the blood glucose levels for the use case of a diabetic user;

FIG. 18A is a depiction of a range of blood glucose levels, and of a total range of blood glucose levels which may occur in different use cases;

FIG. 18B is a depiction of a range of blood glucose levels, and of a total range of blood glucose levels which may occur in different use cases;

FIG. 19 is an example of an output of the computer-implemented invention in the form of a graphical user interface (GUI), for example on a mobile device;

FIG. 20 shows a further flowchart of example steps of the computer-implemented method.

FIG. 21 is an example of an output of the computer-implemented invention in the form of a graphical user interface (GUI), for example on a mobile device;

FIG. 22 is a depiction of a further example of steps of a computer-implemented method.

### DETAILED DESCRIPTION

The detailed description set forth below in connection with the appended drawings is intended as a description of exemplary embodiments of a computer-implemented method provided in accordance with the present invention and is not intended to represent the only forms in which the present invention may be constructed or utilized. The description sets forth the features of the present invention in connection with the illustrated embodiments. It is to be understood, however, that the same or equivalent functions and structures may be accomplished by different embodiments that are also intended to be encompassed within the spirit and scope of the invention. As denoted elsewhere herein, like element numbers are intended to indicate like elements or features.

One or more embodiments of the present invention provide a computer-implemented method for deriving a physiological rank indicative of a physiological status of a user. The computer-implemented method comprises acquiring from a sensor 1101 on a wearable device worn by a user, data including bodily parameter data, for example an optical measurement such as an absorption spectrum related to the user.

The method further comprises applying a model to the bodily parameter data to obtain physiological information related to the user. This information could take the form, for example of information from the spectrum about the physiological status of the user (e.g. from the location/height of the peak). The model derives, from the physiological information, a physiological rank indicative of a physiological status of the user, wherein the physiological rank is a given grade on a physiological rank scale.

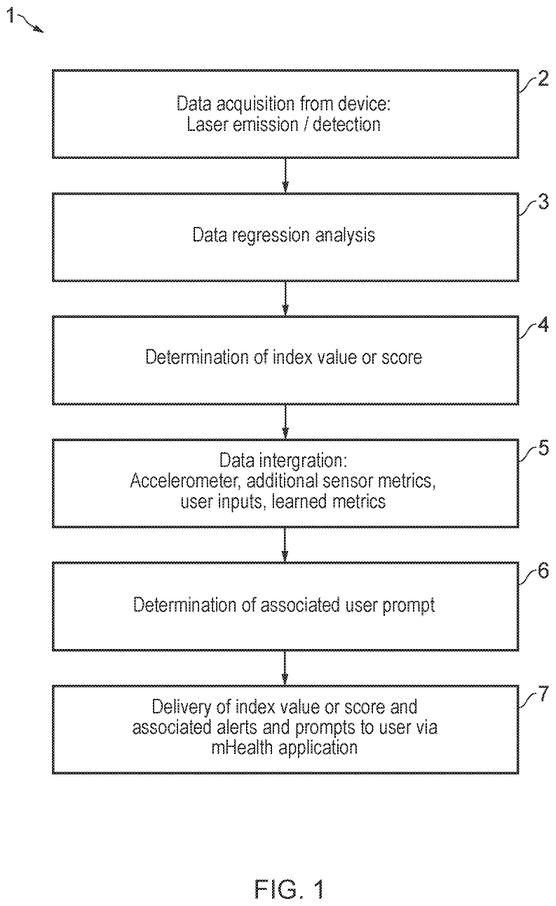

FIG. 1 shows a flow chart 1 setting out steps 2, 3, 4, 5, 6, 7 of the computer-implemented method. Computer-implemented methods according to other embodiments of the present invention may include some, but not all, of the steps shown in FIG. 1. Computer-implemented methods according to other embodiments of the present invention may include additional steps to the steps shown in FIG. 1. The first step 2 of the computer-implemented method shown in FIG. 1 is acquiring data from a sensor on the wearable device.

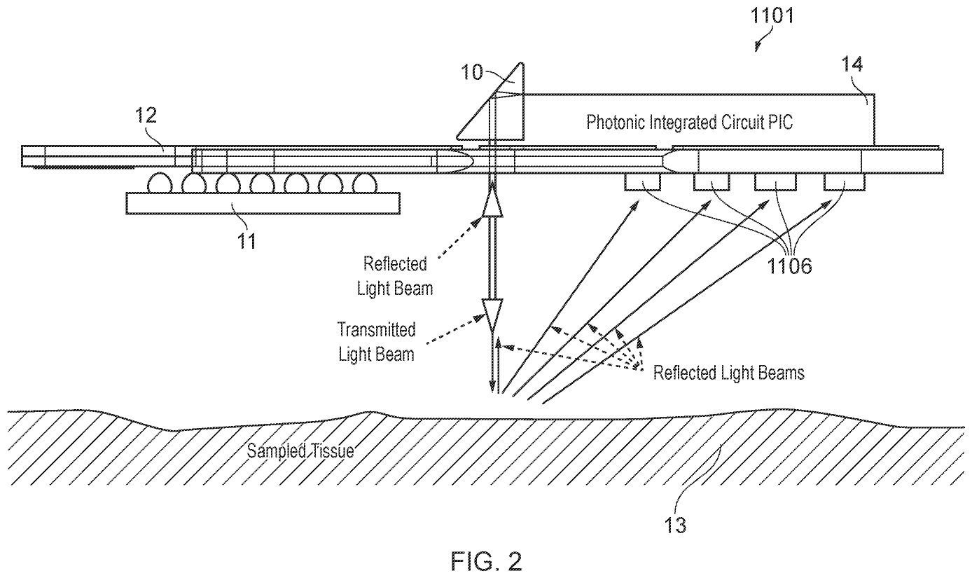

An example of an optical sensing module 1101 will now be described with reference to FIG. 2. The optical sensing module is typically located on the wearable device which acquires the data including the bodily parameter data (e.g. absorption spectrum) related to the user.

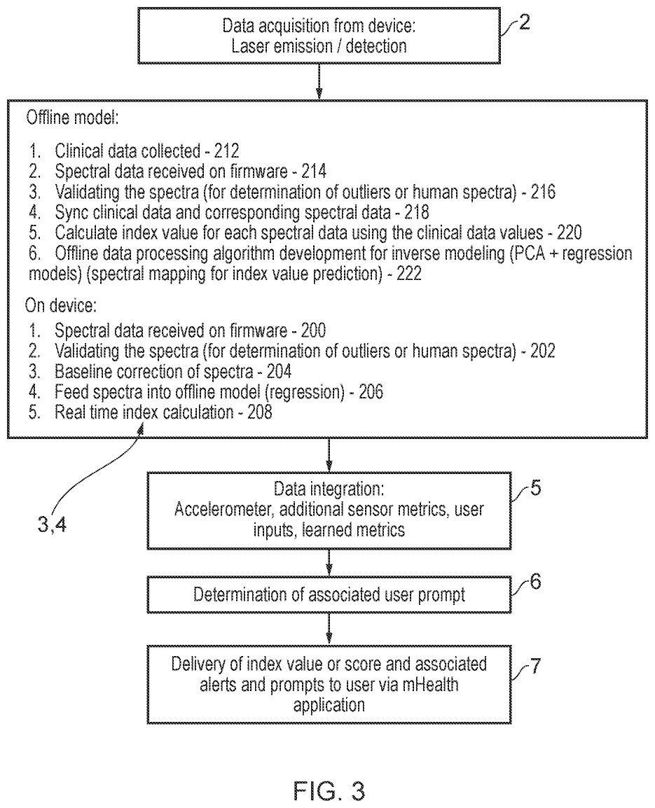

The optical sensing module 1101 includes a transmitter photonic integrated circuit (PIC) 4 located on a substrate 12. The PIC 14 includes a plurality of lasers (not visible in FIG. 3), each laser of the plurality of lasers operating at a wavelength that is different from the wavelength of the others. The optical sensing module 1101 is configured to drive the plurality of lasers one at a time. Light from the plurality of lasers exits the PIC 14 and therefore the optical sensing module 101 via one or more optical output ports. A mirror 10 is present to take the light from the plane of the PIC 14 and translate it into a direction more suitable for interrogating the surface. The direction is orthogonal or substantially orthogonal to the plane of the PIC 14.

The plurality of lasers emit light in a wavelength band which is sensitive to changes in water concentration within the sub corneal interstitial space. The plurality of lasers may emit light in the infrared wavelength band. The plurality of lasers may emit light in the near-infrared wavelength band. The plurality of lasers may emit light in the short wavelength infrared wavelength band. A laser within the plurality of lasers may emit light at 970 nm, 1200 nm, 1450 nm, 1950 nm, 2766 nm, 2898 nm, or 6097 nm, which correspond to water absorption peaks.

In other embodiments, the optical sensing module 1101 may include LEDs in addition to or instead of the lasers.

In use, emitted light from the plurality of lasers is transmitted towards the skin 13 of a user.

Back-scattered light from the surface of the skin 13, and from within a volume below the surface of the skin, returns to the optical sensing module 1101.

A photodetector array comprising photodetector pixels 1106, which collect the backscattered light, forms part of the optical sensing module 1101. In the example shown in FIG. 2, the photodetector array is located on the substrate 12 but is not part of the PIC 4.

An ASIC or microcontroller 11 is located on the substrate 12 of the optical sensing module 1101.

The wearable device carries out the computer-implemented method according to the present invention on a processor (e.g., on a processor of the microcontroller 11 of the wearable device). In other embodiments, an external device such as a mobile phone carries out the computer-implemented method according to the present invention on a processor of the external device.

When the data is acquired from optical sensing module 1110, or from other optical sensing modules, the bodily parameter data is a body tissue absorption spectrum where the absorption is in the water band. The physiological information is a quantitative value associated with the absorption spectrum, for example a wavelength shift of a peak of the absorption spectrum, a height of a peak of the absorption spectrum, or a width of a peak of the absorption spectrum.

In this way, an indication of clinical physiological status of the user can be provided, as the physiological information is sensitive to concentration changes of water within the skin sub-corneal interstitial fluid. As water in the dermis diminishes, the concentration of solutes become higher, thereby changing the degree of water absorption.

Returning to the flow chart 1 shown in FIG. 1, the second and third steps 3, 4 of the computer-implemented method shown in FIG. 1 respectively include carrying out data regression analysis 3 and determining a physiological rank 4. These steps are shown in more detail in FIG. 3.

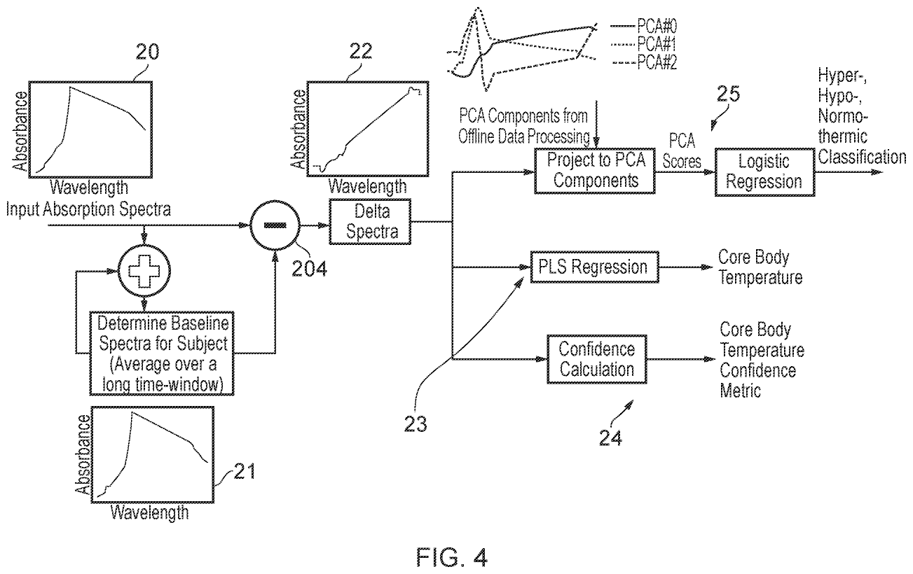

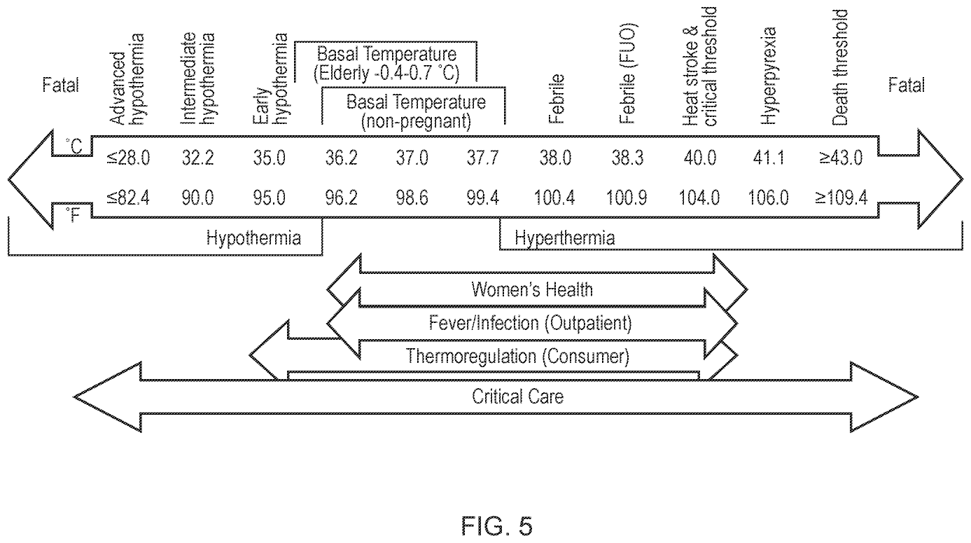

FIG. 3 shows that the second and third steps 3, 4 of the computer-implemented method shown in FIG. 1, include firstly receiving the spectral data 200. Subsequently, pre-processing steps 202, 204 are applied to the data. The first pre-processing step 202 validates the data to determine whether the data has been acquired from a human user, or to determine whether the data includes outlying data. The second pre-processing step 204 applies a baseline correction to the data. This step will be described in more detail below with reference to FIG. 5. Subsequently, the spectra is fed into an offline model 206 and a physiological rank is calculated 208. These steps will be described in more detail below with reference to FIG. 4.

FIG. 4 shows the pre-processing step 204 of applying a baseline correction to the data in more detail. FIG. 4 shows that applying the baseline correction 204 to the data 20 comprises subtracting baseline data 21 from the data 20. The baseline data 21 is the average data acquired from the user over a long time period, for example over 24 hours, 1 week, or 1 month. A delta spectrum 22 results from the baseline correction 21 pre-processing step.

FIG. 4 further shows the step 206 of applying an offline model to the delta spectrum 22 to derive the physiological rank. In the example shown in FIG. 4, the offline model derives, as the physiological rank, both a physiological index and a physiological status of the user. The offline model includes an index model 23 which derives the physiological index, and a status model 25 which derives the physiological status. In the example shown in FIG. 4, the physiological index is a temperature, and the physiological status is a temperature status. The offline model further includes a confidence model 24 which calculates a confidence value for the derived physiological index.

The status model 25 comprises a PCA model and a logistic regression model, where the PCA model is applied to the delta spectrum 22, and the logistic regression model is applied to the output of the PCA model.

The index model 23 is a PLS regression model. The PLS regression model is applied to the delta spectrum.

Further details 210 of how the offline model can generated can be understood with reference to FIG. 3. Each of the index model 23 and the status model 25 of the offline model are generated or trained using training data. The index model and/or the status model may be machine learning models.

A first step 212 outputs of standard clinical tests are received. If the physiological rank is a temperature rank, the standard clinical test may be a test which measure temperature of the user orally using an oral thermometer, for example. In a second step 214 a training dataset is received. A third step 216, is a pre-processing step in which the training dataset is validated to determine whether the training data has been acquired from a human user, or to determine whether the training dataset includes outlying data. In a fourth step 218, the training dataset is mapped to the outputs of the standard clinical tests. In a fifth step 220, a physiological index value is calculated for the training dataset. In a sixth step 222, the offline model is developed.

Thus, training data is collected which comprises a plurality of training datasets, each training dataset being a dataset which comprises bodily parameter information. Each training dataset is associated with a clinical label, where each clinical label is associated with a respective plurality of outputs of standard clinical tests. The clinical data which is used to derive the outputs of the standard clinical tests are acquired at a similar time to the acquisition of the corresponding training dataset, for example within the same 5 minute interval, 15 minute interval or 1 hour interval.

In this way, the offline model effectively maps the data to outputs of standard clinical tests.

The required range of temperatures which can be derived from data acquired by the sensor may vary depending on the use case. As shown in FIG. 5, the required range of temperatures for the use case of women's health may be 35.0° C. to 41.1° C. The required range of temperatures for the use case of infection/fever determination may be 36.0° C. to 40.0° C. The required range of temperatures for the use case of thermoregulation may be 35.0° C. to 40.0° C. The required range of temperatures of the use case of critical care may be 28.0° C. to 43.0° C.

Returning to the computer-implemented method 1 shown in FIG. 1, the fourth step 5 of the computer-implemented method is integrating the data with other sensor information, user-input information or learnt metrics.

At this step, 5 the computer-implemented method comprises acquiring other sensor information. The other sensor information may include, for example, one or more of body temperature information obtained from a temperature sensor, heart rate information obtained from a heart rate sensor, blood oxygen saturation information obtained from a blood oxygen saturation sensor, respiratory rate information obtained from a respiratory rate sensor, hydration information obtained from a hydration sensor, accelerometer and motion information obtained from an accelerometer or a motion sensor, heart rate variability information obtained from a heart rate sensor, alcohol concentration, sleep/wake information obtained from a sleep sensor, blood pressure information obtained from a blood pressure sensor, analyte concentration information (wherein the analyte may be a metabolic fuel such as glucose, lactate, or ethanol) obtained from an analyte concentration sensor, and climate information obtained from a climate sensor (e.g., a thermometer).

User input information may be acquired from a user input into the wearable device or from a user input into an external device such as a mobile phone. The user input information may include, for example, one or more of weight information, height information, activity information, diet information, fluid intake information, sodium intake information, illness information, intoxication information, blood pressure information, sleep duration information, sleep quality information, gender information, age information, heart rate information, cervical mucous information and date of menstruation information.

The computer-implemented method may further comprise a step of acquiring a learnt basal physiological rank or basal bodily parameter data of the user. The basal physiological rank or basal bodily parameter data of the user may be learnt in a calibration period of the computer-implemented method using a machine learning model. The training data for the machine learning model may comprise a plurality of training basal datasets comprising bodily parameter data acquired from the wearable device, and a respective plurality of context labels. Each training basal dataset may be associated with a respective one of the context labels. Each of the context labels may include training other sensor information and/or training user input information. The training other sensor information or user input information is acquired at the same time as or at a similar time to the acquisition of the corresponding training basal dataset, for example within the same 5 minute interval, 15 minute interval or 1 hour interval.

Returning to the computer implemented method 1 shown in FIG. 1 the fifth step 6 of the computer-implemented method is determining a user prompt.

The user prompt which is determined may include a notification, time-correlated hydration information, or an indication that the user's hydration status is outside of a pre-determined basal hydration range. How each of these user prompts is determined will now be described.

To determine a notification, the computer-implemented method comprises storing a notification data table which associates notification with stored physiological ranks. The computer-implemented method comprises comparing the derived physiological rank with the stored physiological ranks respectively and, based on this comparison, selecting a notification to output to the user.

For example, the derived physiological rank may indicate that the user is above their basal temperature. The recommendation associated with this physiological rank may be for the user to drink water.

The notification data table may further associate notifications with stored other sensor information and/or user input information. The computer-implemented method may comprise comparing acquired other sensor information and/or acquired user input information with stored other sensor information and/or stored user input information respectively. Selecting the notification to output to the user may be further based upon this comparison.

For example, if a temperature is derived which is higher than a basal temperature, and user input/other sensor information indicates that the user is hydrated and has not conducted physical activity, the selected notification may indicate to the user that they may have an illness or infection.

To determine a notification, the computer-implemented method may comprise applying a user-notification machine learning model to the health score.

The user-notification machine learning model is trained using training data comprising a plurality of physiological ranks and a respective plurality of context labels. Each of the context labels may comprise user input information and/or other sensor information corresponding to the respective physiological ranks.

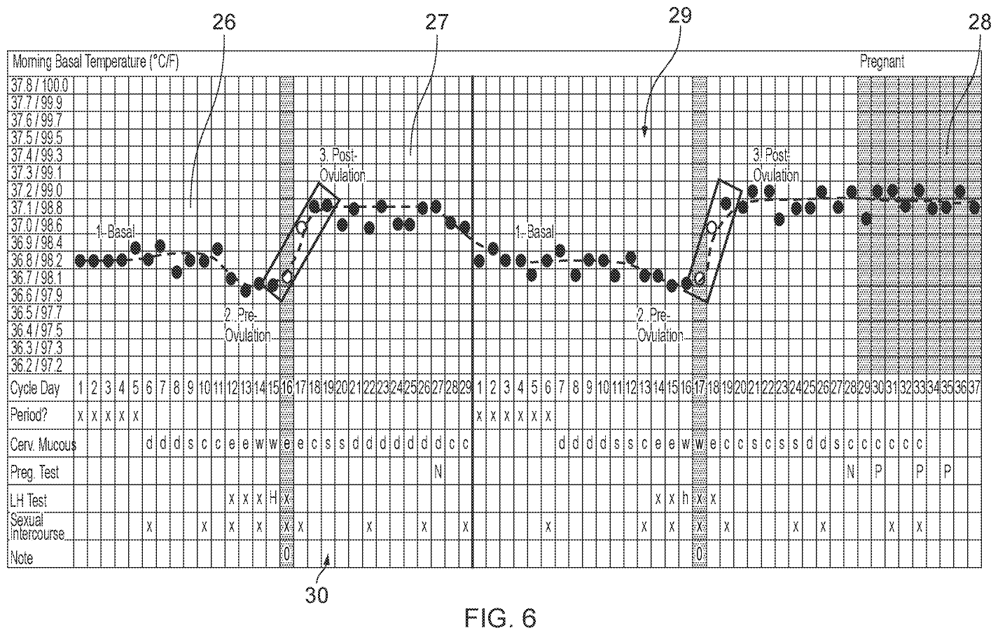

For example, the user-notification machine learning model may select a notification to output to the user based upon a derived temperature of the user and a user input indicative of where the user is in their menstrual cycle, such as their dates of menstruation or their cervical mucous state. As shown in FIG. 6, a user's basal temperature during the luteal phase 27 of their menstrual cycle is 0.2-0.3° C. higher than their basal temperature during the follicular 26 phase of their menstrual cycle. Further, as shown in FIG. 6, a user's basal temperature when they are pregnant 28 may be approximately 0.2-0.3° C. higher than the basal temperature of the user when they are not pregnant 26.

To determine time-correlated physiological information, the computer-implemented method may comprise a step of storing the physiological rank and storing the time at which the physiological rank is derived. The computer-implemented method thus comprises obtaining time-correlated physiological rank information from previously derived stored physiological ranks and their corresponding stored times.

The computer-implemented method may further comprise a step of storing the other sensor information and/or user input information. The computer-implemented method may thus comprise obtaining time-correlated other sensor information and/or time correlated user input information from previously derived stored other sensor information and/or user input information and their corresponding stored times.

To determine whether the user's physiological status is outside of a pre-determined basal physiological range, the computer-implemented method may comprise a step of applying a basal physiological model to the bodily parameter data, or the derived physiological rank.

In one or more embodiments, the basal physiological model takes the learnt basal bodily parameter data or learnt physiological rank as an input. The basal physiological model may derive whether the derived bodily parameter data, or derived physiological rank is more than a pre-determined threshold away from a user's basal bodily parameter data or basal physiological rank respectively.

Returning to the method 1 shown in FIG. 2, the sixth step 7 of the computer-implemented method shown in FIG. 1 is delivery of an output to the user.

The output may be displayed to the user on the wearable device, or on an external device such as a mobile phone.

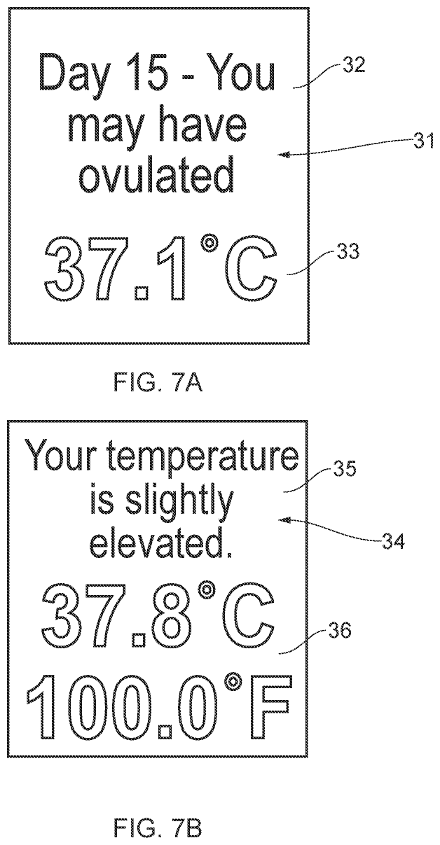

FIGS. 7A and 7B show examples of an outputs 31, 34 which may displayed to a user. The output 31 in FIG. 7A includes a notification to the user that they may have ovulated 32, and the user's temperature in degrees Celsius 65. The output 34 in FIG. 7B includes a notification to the user that their temperature is slightly elevated 35, and the user's temperature in degrees Celsius and degrees Fahrenheit 36.

The output further may further include time-correlated temperature information 29 along with time-correlated user input information 30 and/or other sensor information 30 as shown in FIG. 6.

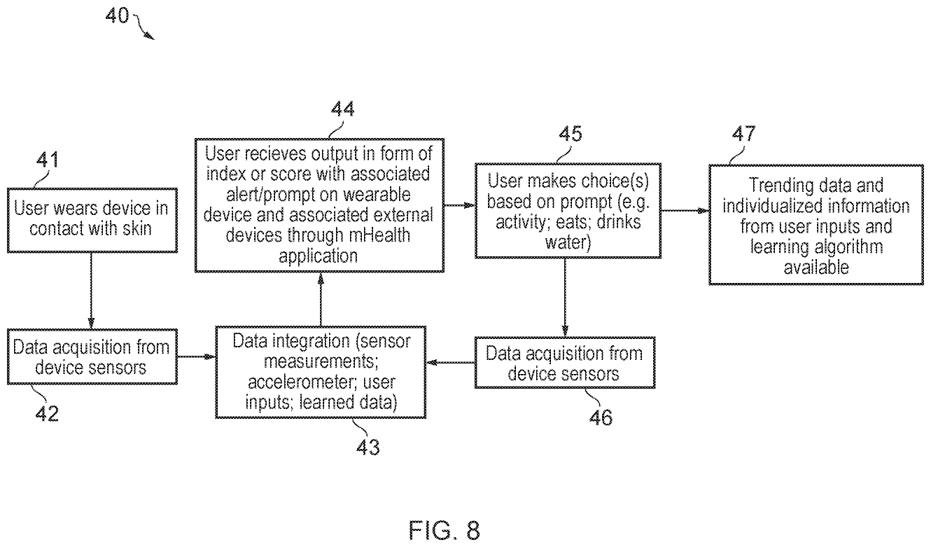

FIG. 8 shows a flow chart 40 for a method which includes embodiments of the computer-implemented method of the present invention. At a first step 41, the user wears a wearable device such that it is in contact with their skin. At a second step 42, data is acquired from sensors of the wearable device. At a third step 43, other sensor information, user input data, and learnt parameters are acquired. At a fourth step 44, an output is provided to the user. At a fifth step 45, the user makes a choice based on the output. At a sixth step 46, data is re-acquired from the sensors. The third step 43 to the sixth step 46 are carried out in a loop. At a seventh step 47, which follows the fifth step, time-correlated information and learnt parameters are acquired.

One or more embodiments of the present invention provide a computer-implemented method for determining a hydration status of a user. The computer-implemented method comprises acquiring from a sensor 1101 on a wearable device worn by a user, data including bodily parameter data, for example an optical measurement such as an absorption spectrum related to the user.

The method further comprises applying a model to the bodily parameter data to obtain hydration information related to the user. This information could take the form, for example of information from the spectrum about how hydrated the user is (e.g. from the location/height of the peak). The model derives, from the hydration information, a hydration rank 1 indicative of a hydration status of the user, wherein the hydration rank 1 is a given grade on a hydration rank scale 51.

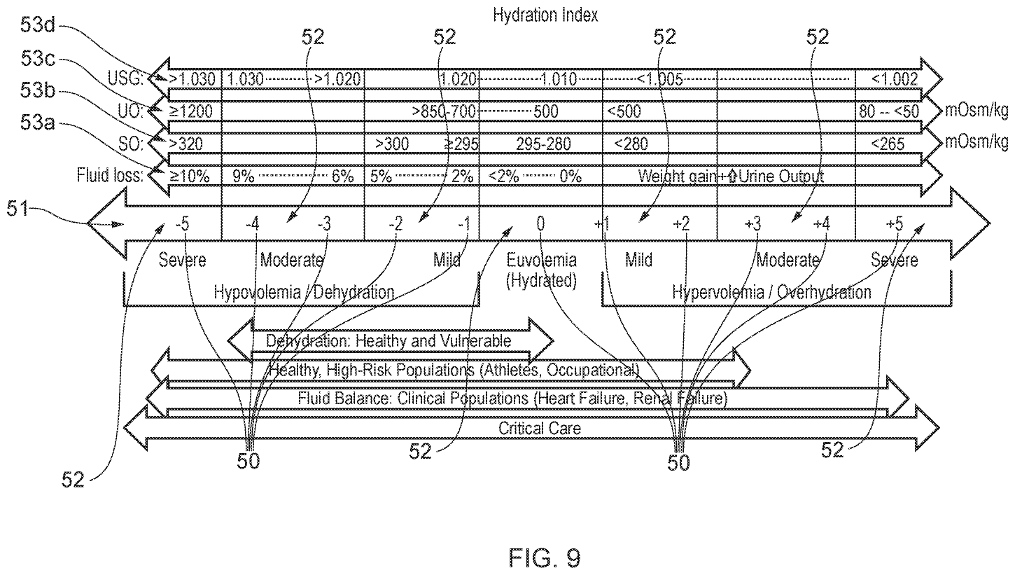

FIG. 9 shows an example of a hydration rank scale 51 according to an embodiment of the present invention. In the embodiment shown in FIG. 9, the hydration rank scale 51 is a scale of hydration indices 10. Each of the hydration indices 50 are integer numbers, and the scale runs from −5 to +5. The model derives a hydration index 50 on this hydration index scale 51.

As shown in FIG. 9, each hydration index 50 on the hydration index scale 51 is indicative of a clinical hydration status of the user. The hydration index 12 scale is sub-divided into a plurality of sub-ranges 52 of hydration index values 1, each of the plurality of sub-ranges 52 corresponding to a different clinical hydration status of the user. These clinical hydration statuses include severe dehydration, moderate dehydration, mild dehydration, euvolemia, mild overhydration, moderate overhydration and severe overhydration. The sub-range 52 including hydration index −5 corresponds to severe dehydration, the sub-range including hydration indices −3 and −4 correspond to moderate dehydration, the sub-range 52 including hydration indices −1 and −2 correspond to mild dehydration, the sub-range 52 including hydration index 0 corresponds to euvolemia, the sub-range 52 including hydration indices +1 and +2 corresponds to mild overhydration, the sub-range including hydration indices +3 and +4 corresponds to moderate overhydration, and the sub-range 52 including hydration index +5 corresponds to severe overhydration.

As further shown in FIG. 9, each hydration index 50 on the hydration index scale 51 maps onto respective ranges of outputs of a plurality of standard clinical point of care tests 53a/b/c/d. In the example shown in FIG. 9, the standard clinical point of care tests 53a/b/c/d are tests of urine osmolality 53c, urine specific gravity 53d, fluid loss 53a and serum osmolality 53b.

Each of these standard clinical point of care tests 53a/b/c/d defines ranges of outputs of the standard clinical point of care test, wherein each of the ranges corresponds to a respective clinical hydration status.

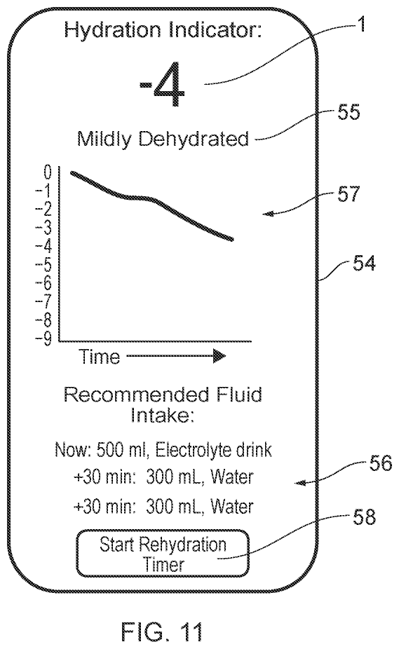

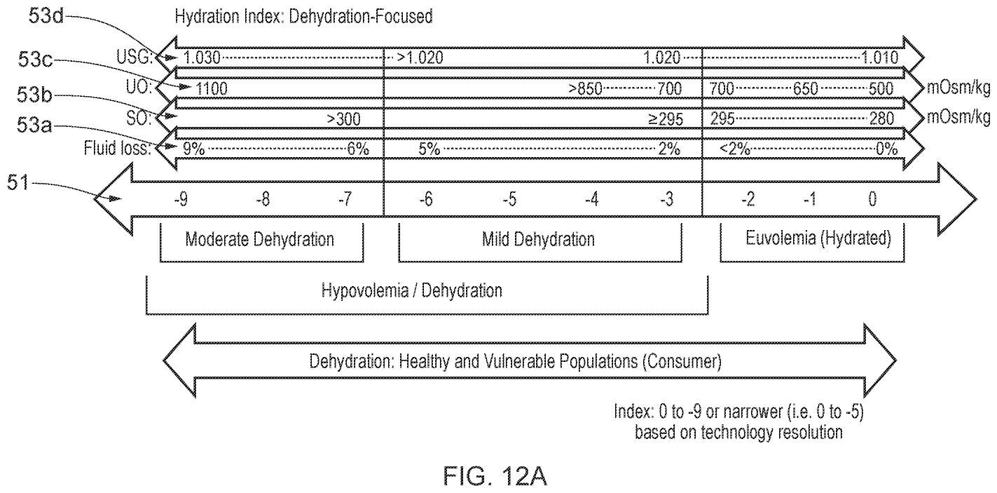

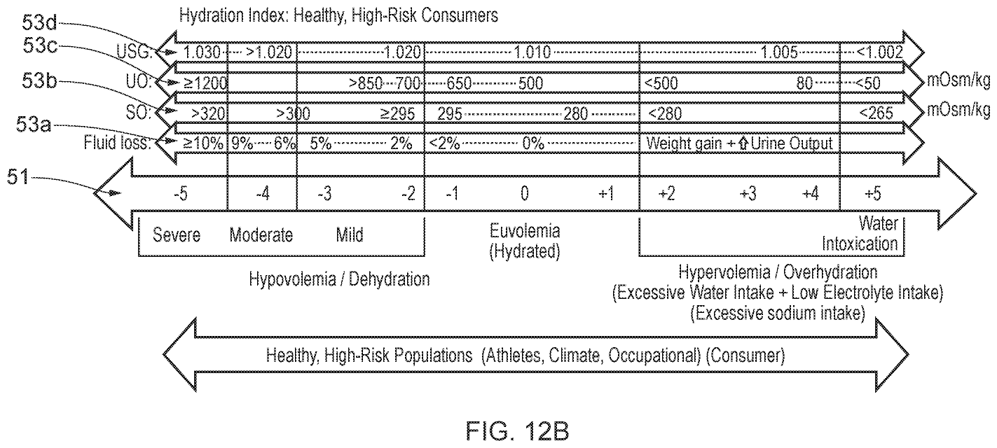

In the example shown in FIG. 11, a hydration index value of −5, which indicates severe dehydration, corresponds to USG values of over 1.030, which also indicates severe dehydration. A sub-range including hydration index values of −3 or −4, which indicate moderate dehydration, corresponds to USG values of between 1.020 and 1.030, which also indicate moderate dehydration. A sub-range including hydration index values of −1 or −2, which indicate mild dehydration, corresponds to USG values of approximately 1.020, which also indicates mild dehydration. A sub-range including hydration index values of −2, −1, 0, 1 or 2, which indicate a hydration status between mild dehydration and mild overhydration, corresponds to USG values of between 1.005 and 1.020, which also indicate a hydration status between mild dehydration and mild overhydration. A hydration index value of 0, which indicates euvolemia corresponds to USG values of approximately 1.010, which also indicates euvolemia. A sub-range including hydration index values of +1 or +2, which indicate mild overhydration, corresponds to a USG value of approximately 1.005, which also indicates mild overhydration. A sub-range including hydration index values of +1, +2, +3, or +4, which indicates a hydration status between mild and moderate overhydration, corresponds to USG values of between 1.002 and 1.005, which also indicates a hydration status between mild and moderate overhydration. A hydration index value of +5, which indicates severe overhydration, corresponds to USG values of less than 1.002, which also indicates severe overhydration.

In other embodiments, the hydration rank scale 51 may be a scale of clinical hydration statuses. In this case, the model derives a clinical hydration status on this clinical hydration status scale. In such embodiments, the clinical hydration statuses (severe dehydration, moderate dehydration, mild dehydration, euvolemia, mild overhydration, moderate overhydration and severe overhydration) shown in FIG. 9, which map onto respective ranges of outputs of the plurality of standard clinical point of care tests, 16a/b/c/d, are directly derived by the model.

FIG. 1 shows a flow chart 1 setting out steps 2, 3, 4, 5, 6, 7 of the computer-implemented method. Computer-implemented methods according to other embodiments of the present invention may include some, but not all, of the steps shown in FIG. 1. Computer-implemented methods according to other embodiments of the present invention may include additional steps to the steps shown in FIG. 1. The first step 2 of the computer-implemented method shown in FIG. 1 is acquiring data from a sensor on the wearable device.

An example of an optical sensing module 1101 will now be described with reference to FIG. 2. The optical sensing module is typically located on the wearable device which acquires the data including the bodily parameter data (e.g. absorption spectrum) related to the user.

The optical sensing module 1101 includes a transmitter photonic integrated circuit (PIC) 4 located on a substrate 12. The PIC 14 includes a plurality of lasers (not visible in FIG. 2), each laser of the plurality of lasers operating at a wavelength that is different from the wavelength of the others. The optical sensing module 1101 is configured to drive the plurality of lasers one at a time. Light from the plurality of lasers exits the PIC 14 and therefore the optical sensing module 101 via one or more optical output ports. A mirror 10 is present to take the light from the plane of the PIC 14 and translate it into a direction more suitable for interrogating the surface. The direction is orthogonal or substantially orthogonal to the plane of the PIC 4.

The plurality of lasers emit light in a wavelength band which is sensitive to changes in water concentration within the interstitial space. The plurality of lasers may emit light in the infrared wavelength band. The plurality of lasers may emit light in the near-infrared wavelength band. The plurality of lasers may emit light in the short wavelength infrared wavelength band. A laser within the plurality of lasers may emit light at 970 nm, 1200 nm, 1450 nm, 1950 nm, 2766 nm, 2898 nm, or 6097 nm, which correspond to water absorption peaks.

In other embodiments, the optical sensing module 1101 may include LEDs in addition to or instead of the lasers.

In use, emitted light from the plurality of lasers is transmitted towards the skin 13 of a user.

Back-scattered light from the surface of the skin 13, and from within a volume below the surface of the skin, returns to the optical sensing module 1101.

A photodetector array comprising photodetector pixels 1106, which collect the backscattered light, forms part of the optical sensing module 1101. In the example shown in FIG. 2, the photodetector array is located on the substrate 12 but is not part of the PIC 4.

An ASIC or microcontroller 11 is located on the substrate 12 of the optical sensing module 1101.

The wearable device carries out the computer-implemented method according to the present invention on a processor (e.g., on a processor of the microcontroller 11 of the wearable device). In other embodiments, an external device such as a mobile phone carries out the computer-implemented method according to the present invention on a processor of the external device.

When the data is acquired from optical sensing module 1110, or from other optical sensing modules, the bodily parameter data is a body tissue absorption spectrum where the absorption is in the water band. The hydration information is a quantitative value associated with the absorption spectrum, for example a wavelength shift of a peak of the absorption spectrum, a height of a peak of the absorption spectrum, or a width of a peak of the absorption spectrum.

In this way, an indication of clinical hydration status of the user can be provided, as the hydration information is sensitive to concentration changes of water within the skin sub-corneal interstitial fluid. As water in the dermis diminishes, the concentration of solutes become higher, thereby changing the degree of water absorption.

Returning to the flow chart 1 shown in FIG. 1, the second and third steps 3, 4 of the computer-implemented method shown in FIG. 1 respectively include carrying out data regression analysis 3 and determining a hydration index value 4. These steps are shown in more detail in FIG. 3.

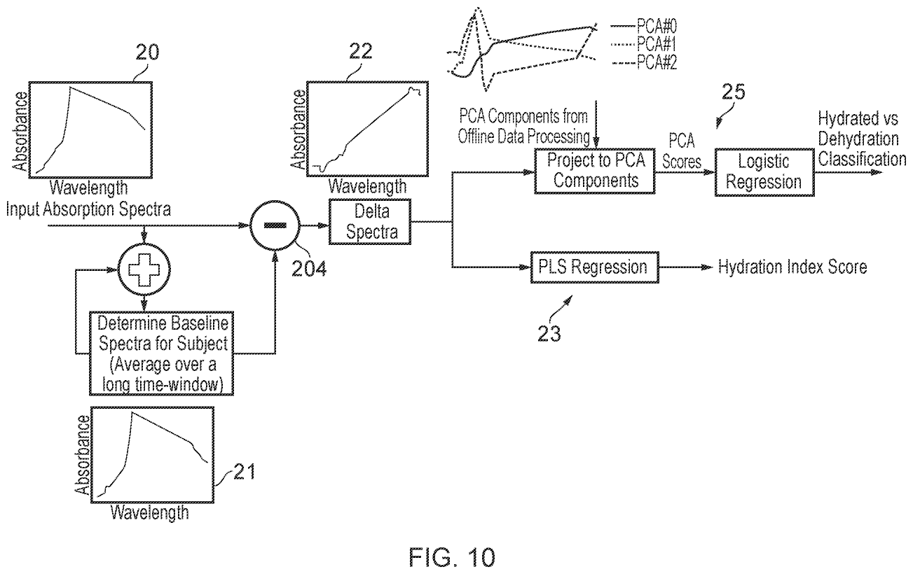

FIG. 3 shows that the second and third steps 3, 4 of the computer-implemented method shown in FIG. 1, include firstly receiving the spectral data 200. Subsequently, pre-processing steps 202, 204 are applied to the data. The first pre-processing step 202 validates the data to determine whether the data has been acquired from a human user, or to determine whether the data includes outlying data. The second pre-processing step 204 applies a baseline correction to the data. This step will be described in more detail below with reference to FIG. 10. Subsequently, the spectra is fed into an offline model 206 and a hydration index value is calculated 208. These steps will be described in more detail below with reference to FIG. 10.

FIG. 10 shows the pre-processing step 204 of applying a baseline correction to the data in more detail. FIG. 10 shows that applying the baseline correction 204 to the data 20 comprises subtracting baseline data 21 from the data 20. The baseline data 21 is the average data acquired from the user over a long time period, for example over 24 hours, 1 week, or 1 month. A delta spectrum 22 results from the baseline correction 21 pre-processing step.

FIG. 10 further shows the step 206 of applying an offline model to the delta spectrum 22 to derive the hydration rank 1. In the example shown in FIG. 10, the offline model derives, as the hydration rank 1, both a hydration index and a hydration status of the user. The offline model includes an index model 23 which derives the hydration index, and a status model 25 which derives the hydration status.

The status model 25 comprises a PCA model and a logistic regression model, where the PCA model is applied to the delta spectrum 22, and the logistic regression model is applied to the output of the PCA model.

The index model 23 is a PLS regression model. The PLS regression model is applied to the delta spectrum.

Further details 210 of how the offline model can generated can be understood with reference to FIG. 3. Each of the index model 23 and the status model 25 of the offline model are generated or trained using training data. The index model and/or the status model may be machine learning models.

A first step 212 outputs of standard clinical point of care tests are received, and in a second step 214 a training dataset is received. A third step 216, is a pre-processing step in which the training dataset is validated to determine whether the training data has been acquired from a human user, or to determine whether the training dataset includes outlying data. In a fourth step 218, the training dataset is mapped to the outputs of the standard clinical point of care tests. In a fifth step 220, a hydration index value is calculated for the training dataset. In a sixth step 222, the offline model is developed.

Thus, training data is collected which comprises a plurality of training datasets, each training dataset being a dataset which comprises bodily parameter information. Each training dataset is associated with a clinical label, where each clinical label is associated with a respective plurality of outputs of standard clinical point of care tests. The clinical data which is used to derive the outputs of the standard clinical point of care tests are acquired at a similar time to the acquisition of the corresponding training dataset, for example within the same 5 minute interval, 15 minute interval or 1 hour interval.

In this way, the offline model effectively maps the data to outputs of standard point of care tests.

Returning to the computer-implemented method 1 shown in FIG. 1, the fourth step 5 of the computer-implemented method is integrating the data with other sensor information, user-input information or learnt metrics.

At this step, 5 the computer-implemented method comprises acquiring other sensor information. The other sensor information may include, for example, body temperature obtained from a temperature sensor, activity information obtained from an accelerometer, heart rate information obtained from a heart rate sensor and blood pressure information obtained from a blood pressure sensor.

User input information may be acquired from a user input into the wearable device or from a user input into an external device such as a mobile phone. The user input information may include, for example, weight information, activity information, diet information, fluid intake information, illness information and intoxication information.

The computer-implemented method may further comprise a step of acquiring a learnt basal hydration rank or basal bodily parameter data of the user. The basal hydration rank or basal bodily parameter data of the user may be learnt in a calibration period of the computer-implemented method using a machine learning model. The training data for the machine learning model may comprise a plurality of training basal datasets comprising bodily parameter data acquired from the wearable device, and a respective plurality of context labels. Each training basal dataset may be associated with a respective one of the context labels. Each of the context labels may include training other sensor information and/or training user input information. The training other sensor information or user input information is acquired at the same time as or at a similar time to the acquisition of the corresponding training basal dataset, for example within the same 5 minute interval, 15 minute interval or 1 hour interval.

Returning to the computer implemented method 1 shown in FIG. 1, the fifth step 6 of the computer-implemented method is determining a user prompt.

The user prompt which is determined may include a cause of a hydration status, a recommendation, a type of rehydration fluid to consume, a volume of rehydration fluid for the user to consume, a rehydration schedule for the user to follow, time-correlated hydration information, an indication that the user's hydration status is outside of a pre-determined basal hydration range, or an indication that the user remains dehydrated or overhydrated after a reassessment time. How each of these user prompts is determined will now be described.

To determine the cause of a hydration status, the computer-implemented method may comprise storing a hydration status cause data table which associates causes of a hydration status with stored other sensor information and/or stored user input information. The computer-implemented method may comprise, when a hydration rank is determined which indicates that the user's clinical hydration status is a pre-determined clinical hydration status, comparing acquired other sensor information and/or user input information with stored other sensor information and/or user input information respectively, and based on this comparison, selecting a cause of a clinical hydration status.

The pre-determined clinical hydration status may be dehydration or overhydration.

For example, the derived hydration index may indicate that the user is dehydrated, and the other sensor information may include temperature information which indicates that the user has a high temperature. The cause of dehydration associated with this temperature information may be, for example, that the user is ill, or that the user is overheated.

To determine a recommendation, the computer-implemented method comprises storing a recommendation data table which associates recommendations with stored hydration ranks. The computer-implemented method comprises comparing the derived hydration rank with the stored hydration ranks respectively and, based on this comparison, selecting a recommendation to output to the user.

For example, the derived hydration rank may indicate that the user is dehydrated. The recommendation associated with this hydration rank may be for the user to drink water.

The recommendation data table may further associate recommendations with stored other sensor information and/or user input information. The computer-implemented method may comprise comparing acquired other sensor information and/or acquired user input information with stored other sensor information and/or stored user input information respectively. Selecting the recommendation to output to the user may be further based upon this comparison.

For example, the derived hydration rank may indicate that the user is dehydrated, and activity information obtained from an accelerometer may indicate that the user is doing/has just finished doing exercise. The recommendation associated with this activity information for this hydration rank may be to rest, or to consume electrolyte fluid.

To determine a type of rehydration fluid to consume, the computer-implemented method comprises storing a rehydration fluid type data table. The rehydration fluid type data table associates types of rehydration fluids with stored other sensor information and/or stored user input information.

The computer-implemented method comprises, when a hydration rank is derived which indicates that the clinical hydration status of the user is dehydration, comparing acquired other sensor information and/or acquired user input information with stored other sensor information and/or stored user input information respectively and, based on this comparison, selecting a type of rehydration fluid.

For example, the derived hydration rank may indicate that the user is dehydrated, and activity information obtained from an accelerometer may indicate that the user is doing/has just finished doing exercise. The rehydration fluid selected in this case may be an electrolyte fluid.

To determine a volume of rehydration fluid for the user to consume, the computer-implemented method comprises storing a rehydration fluid volume data table which associates volumes of rehydration fluid with stored hydration ranks.

The computer-implemented method comprises, when a hydration rank is derived which indicates that the clinical hydration status if the user is dehydration, comparing a derived hydration rank with stored hydration ranks and, based on this comparison, selecting a volume of rehydration fluid.

The rehydration fluid volume data table may further associate volumes of rehydration fluid with stored other sensor information and/or user input information.

The computer-implemented method may comprise, when a hydration rank is derived which indicates that the clinical hydration status of the user is dehydration, comparing acquired other sensor information with stored other sensor information and, based on this comparison, selecting a volume of rehydration fluid. Alternatively, or in addition, the computer-implemented method may comprise, when a hydration rank is derived which indicates that the clinical hydration status of the user is dehydration, comparing acquired user input information with stored user input information and, based on this comparison, selecting a volume of rehydration fluid.

To determine a rehydration schedule for the user to follow, the computer-implemented method may comprise storing a rehydration schedule data table which associates a schedule by which rehydration fluid should be consumed with stored hydration ranks.

The computer-implemented method may comprise, when a hydration rank is derived which indicates that the clinical hydration status of the user is dehydration, comparing a derived hydration rank with stored hydration ranks and based on this comparison, selecting a schedule by which rehydration fluid should be consumed.

The rehydration fluid volume data table may further associate a schedule by which rehydration fluid should be consumed with stored user input information and/or other sensor information.

The computer-implemented method may comprise, when a hydration rank is derived which indicates that the clinical hydration status of the user is dehydration, comparing acquired other sensor information or user input information with stored other sensor information or user input information respectively and, based on this comparison, selecting a schedule by which rehydration fluid should be consumed.

For example, the hydration rank may indicate that the user is moderately dehydrated and temperature information acquired from a temperature sensor may indicate that the user is overheated. The selected schedule by which rehydration fluid should be consumed may be selected based on these factors. The schedule may include a type of rehydration fluid to consume, an overall volume of rehydration fluid to consume, sub-volumes of the overall volume of rehydration fluid to consume and times at which the sub-volumes of rehydration fluid should be consumed.

To determine time-correlated hydration information, the computer-implemented method may comprise a step of storing the hydration rank and storing the time at which the hydration rank is derived. The computer-implemented method thus comprises obtaining time-correlated hydration rank information from previously derived stored hydration ranks and their corresponding stored times.

The computer-implemented method may further comprise a step of storing the other sensor information and/or user input information. The computer-implemented method may thus comprise obtaining time-correlated other sensor information and/or time correlated user input information from previously derived stored other sensor information and/or user input information and their corresponding stored times.

To determine whether the user's hydration status is outside of a pre-determined basal hydration range, the computer-implemented method may comprise a step of applying a basal hydration model to the bodily parameter data, or the derived hydration rank.

In one or more embodiments, the basal hydration model takes the learnt basal bodily parameter data or learnt hydration rank as an input. The basal hydration model may derive whether the derived bodily parameter data, or derived hydration rank is more than a pre-determined threshold away from a user's basal bodily parameter data or basal hydration rank respectively.

To determine whether the user remains dehydrated or overhydrated after a reassessment time, the computer-implemented method comprises re-deriving the hydration rank after a reassessment time. The reassessment time may be a pre-determined reassessment time, a user-input reassessment time, or a reassessment time based upon the initially derived hydration rank.

Returning to the method 1 shown in FIG. 1, the sixth step 7 of the computer-implemented method shown in FIG. 1 is delivery of an output to the user.

The output may be displayed to the user on the wearable device, or on an external device such as a mobile phone.

FIG. 11 shows an example of an output being displayed to a user on a mobile device which is a mobile phone 54. The output includes a derived hydration index 50 of −4, and a derived clinical hydration status 55 of mildly dehydrated.

The output further includes a recommended rehydration schedule 56 which is to drink 500 ml of electrolyte fluid at a current time, 300 ml of water after half an hour, and another 300 ml of water after an hour.

The output also includes time-correlated hydration information 57 which shows that the user's hydration index has fallen from 0 to −4 over a period of time.

FIG. 11 also shows that a user input may be received on a portion 58 of the user interface of the mobile device which displays “Start Rehydration Timer” to the user to cause the computer-implemented method to re-derive the hydration status or the hydration index of the user after a pre-determined reassessment time. The computer-implemented method may comprise, when a hydration status or a hydration index, derived after the reassessment time, indicates that the user remains dehydrated after the reassessment time, outputting an alert to the user.

FIG. 8 shows a flow chart 40 for a method which includes embodiments of the computer-implemented method of the present invention. At a first step 41, the user wears a wearable device such that it is in contact with their skin. At a second step 42, data is acquired from sensors of the wearable device. At a third step 43, other sensor information, user input data, and learnt parameters are acquired. At a fourth step 44, an output is provided to the user. At a fifth step 45, the user makes a choice based on the output. At a sixth step 46, data is re-acquired from the sensors. The third step 43 to the sixth step 46 are carried out in a loop. At a seventh step 47, which follows the fifth step, time-correlated information and learnt parameters are acquired.

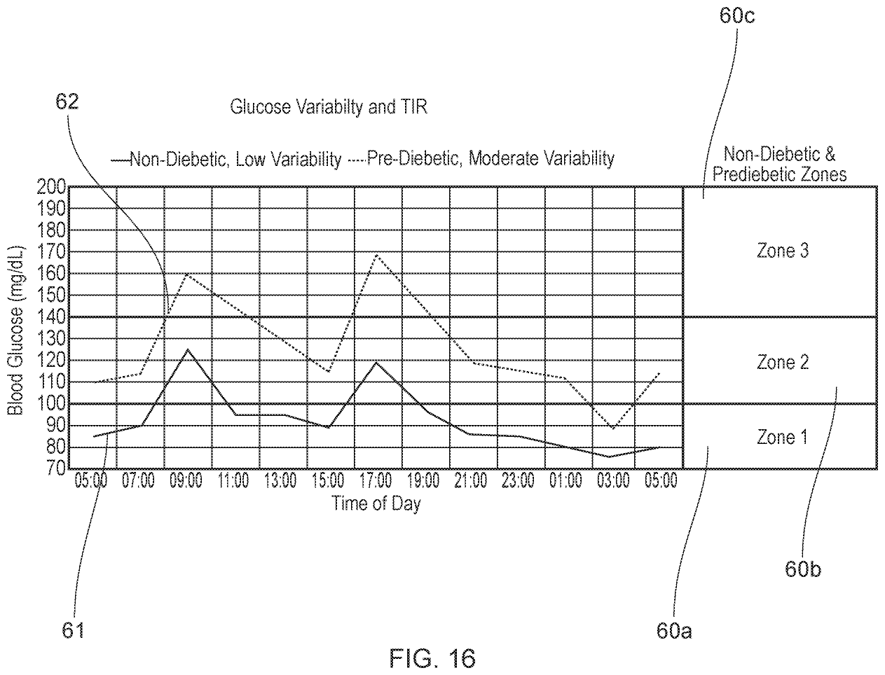

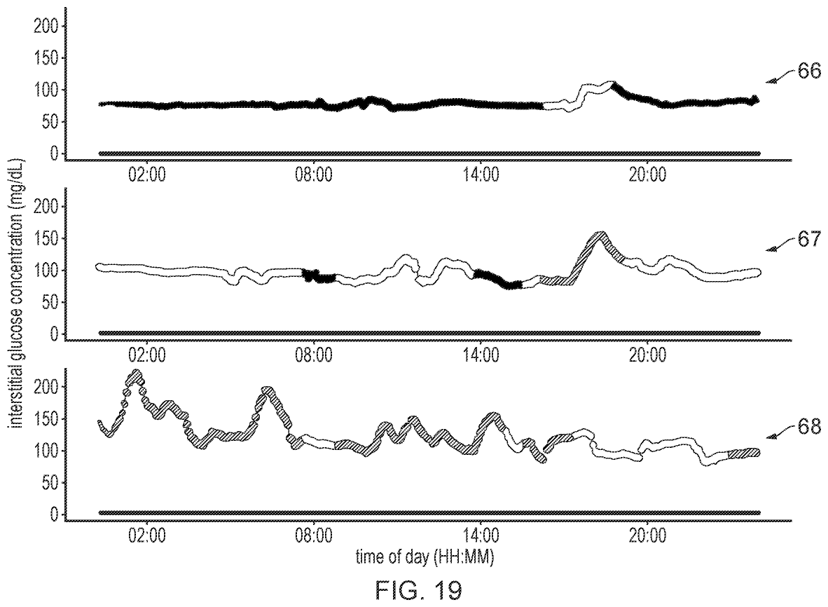

FIGS. 16 and 16 show examples of different hydration index scales 12 to that shown in FIG. 19. As shown in FIGS. 9, 16A and 16B, the hydration index scale 51, and how its hydration indices 50 map onto output of standard clinical point of care tests 16a/b/c/d, may depend on the use case for the computer-implemented method. For example, FIG. 19 shows a hydration index scale 51 running from −5 to +5 which may be used for critical care patients. FIG. 16A shows a hydration index scale 51 running from −9 to 0 which may be used for both healthy and vulnerable users. FIG. 16B shows a hydration index scale 51 running from −5 to +5 which may be used for healthy, high-risk users such as athletes. Populations that may benefit from the present invention include elderly populations, endurance athletes, those travelling to altitude or hot climates, and those in occupations with a high risk of dehydration and overheating.

One or more embodiments of the present invention provide a computer-implemented method for determining how well a user's body is regulating an analyte. The computer-implemented method comprises acquiring from a sensor 1101 on a wearable device worn by a user, data including bodily parameter data related to the user, for example an optical measurement such as a diffuse reflectance spectrum related to the user.

The method further comprises applying a model to the bodily parameter data to obtain analyte concentration information related to the user. The information could take the form, for example, of information from the spectrum about the levels of an analyte such as glucose in the user's blood (e.g., from the location/height of the peak). The model derives, from the analyte concentration information, a health score indicative of how well the user's body is regulating the analyte, wherein the health score is a given grade on a health score scale.

FIG. 1 shows a flow chart 1 setting out steps 2, 3, 4, 5, 6, 7 of the computer-implemented method. Computer-implemented methods according to other embodiments of the present invention may include some, but not all, of the steps shown in FIG. 1. Computer-implemented methods according to other embodiments of the present invention may include additional steps to the steps shown in FIG. 1. The first step 2 of the computer-implemented method shown in FIG. 1 is acquiring data from a sensor on the wearable device.

An example of an optical sensing module 1101 will now be described with reference to FIG. 2. The optical sensing module 1101 is typically located on the wearable device which acquires the data including the bodily parameter data (e.g. absorption spectrum) related to the user.

The optical sensing module 1101 includes a transmitter photonic integrated circuit (PIC) 4 located on a substrate 12. The PIC 14 includes a plurality of lasers (not visible in FIG. 2) each laser of the plurality of lasers operating at a wavelength that is different from the wavelength of the others. The optical sensing module 1101 is configured to drive the plurality of lasers one at a time. Light from the plurality of lasers exits the PIC 14 and therefore the optical sensing module 101 via one or more optical output ports. A mirror 10 is present to take the light from the plane of the PIC 14 and translate it into a direction more suitable for interrogating the surface. The direction is orthogonal or substantially orthogonal to the plane of the PIC 4.

The plurality of lasers emit light in a wavelength band which is sensitive to changes in sensitive to changes in concentrations of the analyte for which the analyte concentration value is determined. The plurality of lasers may emit light in the infrared wavelength band. The plurality of lasers may emit light in the near-infrared wavelength band. The plurality of lasers may emit light in the short wavelength infrared wavelength band.

In other embodiments, the optical sensing module 1101 may include LEDs in addition to or instead of the lasers.

In use, emitted light from the plurality of lasers is transmitted towards the skin 13 of a user.

Back-scattered light from the surface of the skin 13, and from within a volume below the surface of the skin at a depth at which sufficient blood is present for sensing the presence of the analyte, returns to the optical sensing module 1101.

A photodetector array comprising photodetector pixels 1106, which collect the backscattered light, forms part of the optical sensing module 1101. In the example shown in FIG. 2, the photodetector array is located on the substrate 12 but is not part of the PIC 4.

An ASIC or microcontroller 11 is located on the substrate 12 of the optical sensing module 1101.

The wearable device carries out the computer-implemented method according to the present invention on a processor (e.g., on a processor of the microcontroller 11 of the wearable device). In other embodiments, an external device such as a mobile phone carries out the computer-implemented method according to the present invention on a processor of the external device.

When the data is acquired from optical sensing module 1110, or from other optical sensing modules, the bodily parameter data is an infrared diffuse reflectance spectrum. The analyte concentration information is a quantitative value associated with the infrared diffuse reflectance spectrum, for example a wavelength shift of a peak of the reflectance spectrum, a height of a peak of the reflectance spectrum, or a width of a peak of the reflectance spectrum.

In this way, an indication of how well the user's body is regulating the analyte in the blood can be provided, as the analyte concentration information is sensitive to concentration changes of the analyte in the blood.

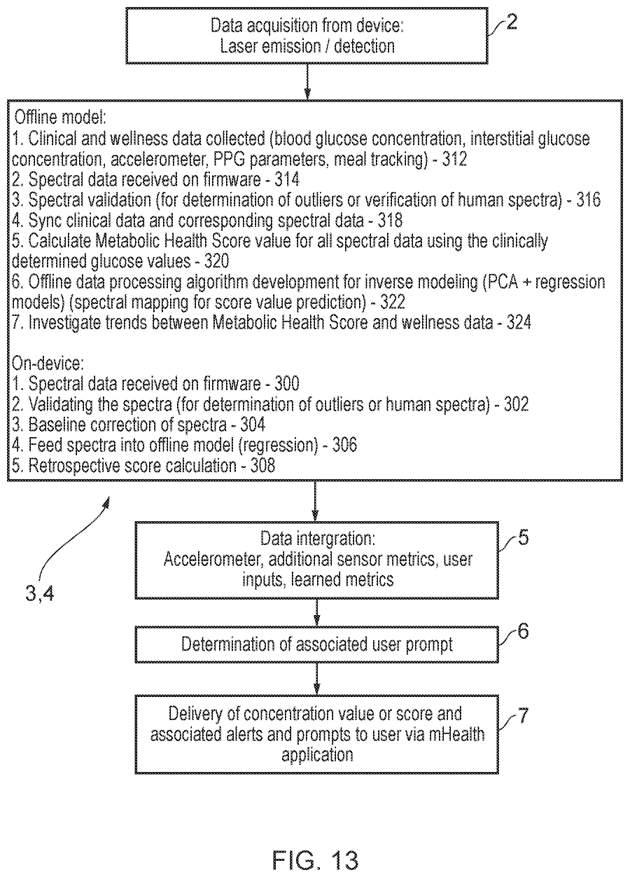

Returning to the flow chart 1 shown in FIG. 1 the second and third steps 3, 4 of the computer-implemented method shown in FIG. 1 respectively include carrying out data regression analysis 3 and determining a health score 4. These steps are shown in more detail in FIG. 13.

FIG. 13 shows that the second and third steps 3, 4 of the computer-implemented method shown in FIG. 1, include firstly receiving the data 300. Subsequently, pre-processing steps 302, 304 are applied to the data. The first pre-processing step 302 validates the data to determine whether the data has been acquired from a human user, or to determine whether the data includes outlying data. The second pre-processing step 304 applies a baseline correction to the data. Subsequently, the spectra is fed into an offline model 306 and a health score is calculated 3208. These steps 300, 302, 304, 306, 308 are further depicted in FIG. 4.

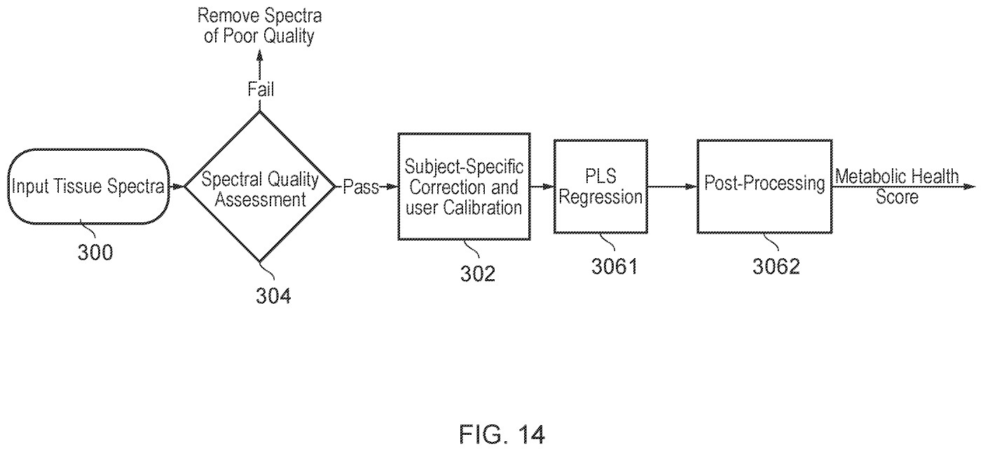

FIG. 14 shows the steps of receiving the spectral data 300, of validating the data 302 and of applying a baseline correction to the data 304.

FIG. 14 further shows the step 3061 of applying a PLS regression model to the data output by the pre-processing step 304. The PLS regression model forms a part of the offline model. The PLS regression model outputs an analyte concentration value.

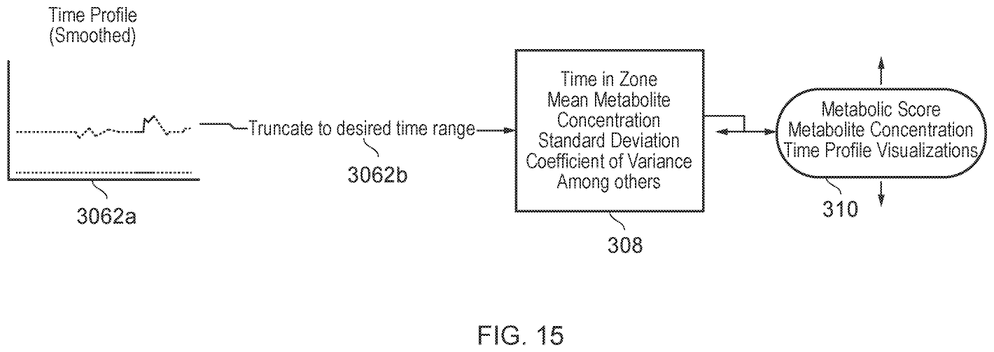

FIG. 14 further shows that a step 3062 of applying post-processing to outputs of the PLS regression model is carried out, and that subsequently the health score is calculated. The post-processing step 3062 and the calculation of the health score is shown in more detail in FIG. 15.

In the embodiment depicted in FIG. 15 the offline model is applied to a plurality of datasets acquired from the sensor on the wearable device at different times, and a respective plurality of analyte concentration values are derived by the PLS regression model.

FIG. 15 shows that the first post-processing step 3062a is smoothing the time profile of the respective plurality of analyte concentration values. The second post-processing step 3062b, is truncating the time profile of the plurality of analyte concentration values to a desired time range.

After the post-processing steps 3062a, 3062b are carried out, the health score is derived 308. As shown in FIG. 15, there are multiple methods by which the health score can be derived. These methods include calculating a duration in which a user's analyte concentration values remain within a given sub-range, calculating a mean analyte concentration value, and calculating a variability of a user's analyte concentration values. These methods will now be discussed in more detail.

To calculate the health score from a duration in which the user's analyte concentration values remain within a given sub-range, the computer-implemented method includes the following steps.

The computer-implemented method includes, for each derived analyte concentration value in the plurality of derived analyte concentration values, determining a sub-range (or zone) of analyte concentration values which the derived analyte concentration value falls within. The computer-implemented method further includes acquiring a plurality of time stamps indicative of the respective plurality of times at which each of the datasets in the plurality of datasets are acquired. The computer-implemented method further includes calculating, using the plurality of sub-ranges and the plurality of time stamps, a duration of time in which a user's analyte concentration remains within a given sub-range. This duration is used to derive the health score.

For example, if a user's analyte concentration remains within a sub-range (or zone) which is indicative of high analyte concentration for a long time, a health score may be derived which indicates that a user's body is sub-optimally regulating the analyte. If a user's analyte concentration remains within a sub-range (or zone) which is indicative of an ideal analyte concentration for a long time, a health score may be derived which indicates that a user's body is optimally regulating the analyte. This concept is demonstrated in FIG. 16.

FIG. 16 is a graph of blood glucose levels against time. The blood glucose levels are divided into zones 60a/b/c. Zone 1 60a indicates ideal blood glucose levels during sleep and fasting. In a 24 hour period, the ideal duration that a user's blood glucose levels are in zone 1 60a is approximately greater than 19 hours. Zone 2 60b indicates ideal blood glucose levels within a 2 hour period after a meal. In a 24 hour period, the ideal duration that a user's blood glucose levels are in zone 2 60b is less than approximately less than 5 hours. Zone 3 60c indicates hyperglycemia (high blood glucose levels). In a 24 hour period, the ideal duration that a user's blood glucose levels are in zone 3 60c may be less than 30 minutes.

FIG. 16 shows a plot of the blood glucose levels of a non-diabetic person (the lower line 61), and a plot of the blood glucose levels of a diabetic person (the upper line 62). As shown in FIG. 4, the blood glucose levels of the diabetic person spend a longer duration in zones 2 and 3 than the blood glucose levels of the non-diabetic person. The blood glucose levels of the non-diabetic person spend a longer duration in zone 1 than the blood glucose levels of the diabetic person.

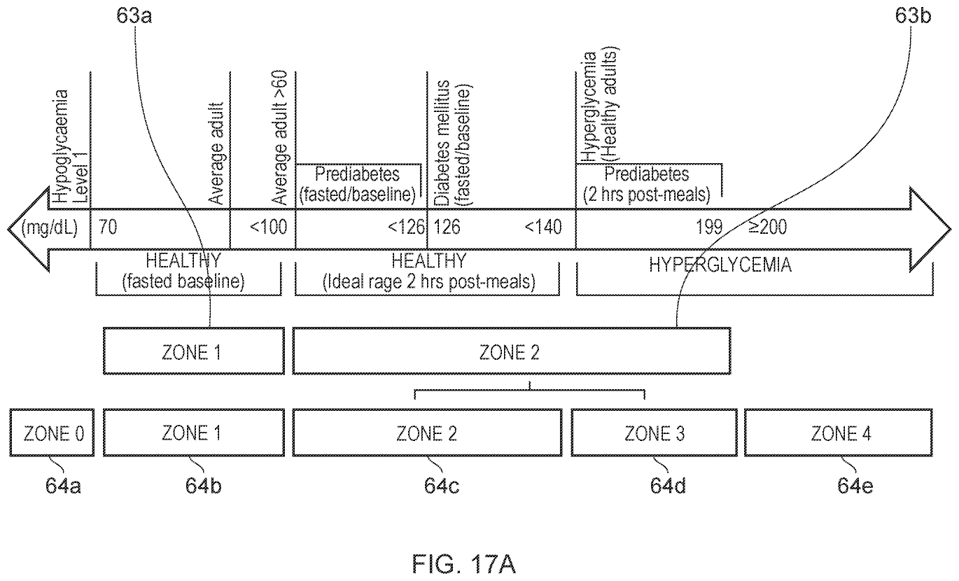

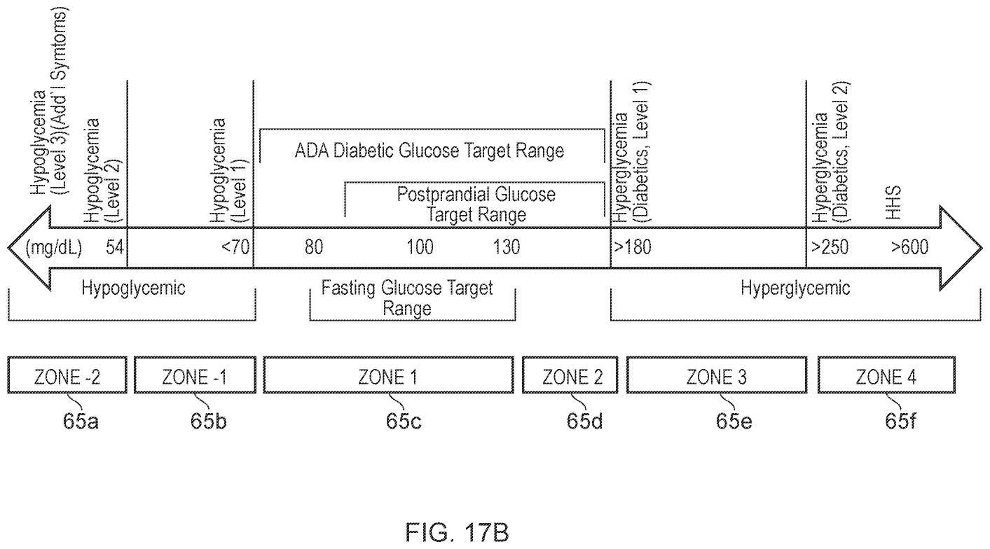

The sub-ranges of the blood glucose levels which define the zones, and the number of zones used in the computer-implemented method may vary based on the use case and the utility of the computer-implemented method. FIG. 17A and FIG. 17B show the number of zones and the sub-ranges of the blood glucose levels which define the zones for non-diabetic users and diabetic users respectively. FIG. 17A shows 2 zones 63a/b, which can be applied when the computer-implemented method is utilized only in fasting periods. FIG. 17A further shows 5 zones 64a/b/c/d/e, which can be applied when the computer-implemented method is utilized in both fasting periods and meal periods, or which can be applied when the computer-implemented method is utilized over a 24-hour period. FIG. 17B shows 6 zones 65a/b/c/d/e/f.

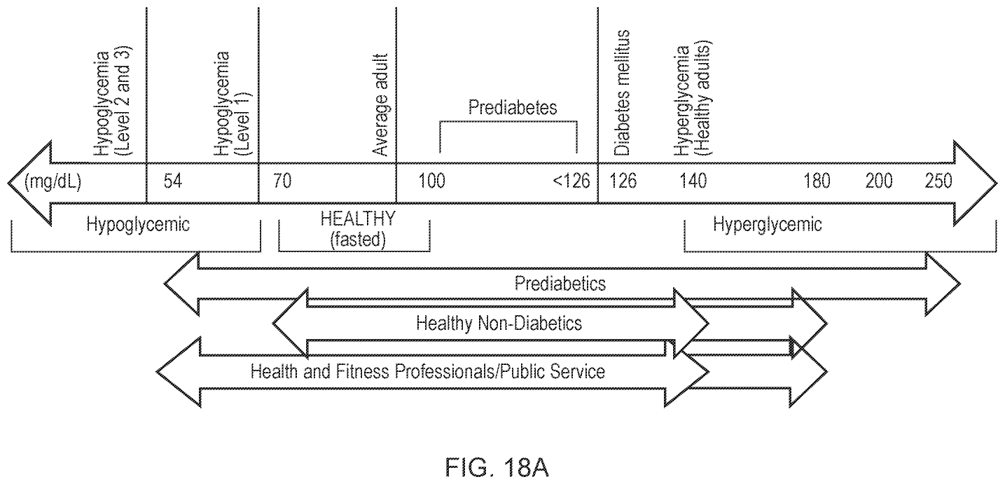

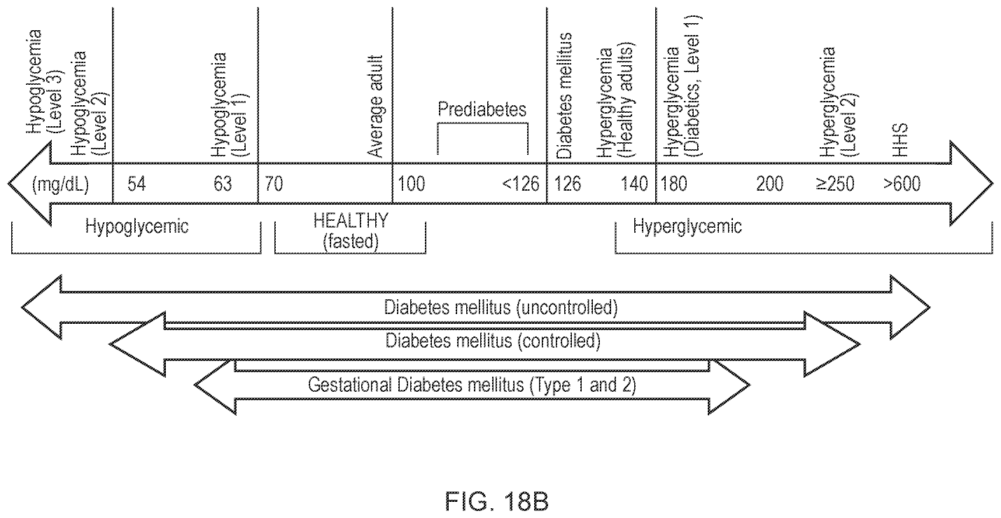

FIG. 18A and FIG. 18B show total ranges of glucose levels which may occur in different use cases of prediabetic users, healthy users, health and fitness users or public service users, users with uncontrolled diabetes, users with controlled diabetes, and users with gestational diabetes. It may be useful to be able to obtain data from the sensor across these total ranges in each of these use cases.

To calculate the health score from a mean analyte concentration value, the computer-implemented method includes calculating, using the plurality of analyte concentration values, a mean of the plurality of analyte concentration values.

To calculate the health score from the variability of the user's analyte concentration values, the computer-implemented method includes calculating, using the plurality of analyte concentration values, a variability of the plurality of analyte concentration values. The variability may be a standard deviation, a coefficient of variation, a mean amplitude glucose excursion, a J-index, a mean absolute difference, or a mean absolute glucose.

A higher variability may indicate that the user's body is not regulating the analyte as well, and thus a higher variability may result in a lower health score. A lower variability may indicate that the user's body is regulating the analyte better, and thus a lower variability may result in a higher health score. This concept is demonstrated in FIG. 19.

FIG. 19 shows three graphs 66, 67, 68 of blood glucose levels against time. The top graph 66 shows blood glucose levels which are well regulated, the middle graph 67 shows blood glucose levels which are moderately well regulated, and the bottom graph 68 shows blood glucose levels which are badly regulated. The variability of the blood glucose levels increases as the blood glucose levels become less well regulated (i.e., the variability is smallest in the top graph, and is greatest in the bottom graph).

FIG. 15 further shows that the computer-implemented method includes outputting various outputs, which may include the health score, a mean analyte concentration and time-correlated information. The outputs are discussed in more detail below with reference to FIG. 19.

Details of how the offline model can generated can be understood with reference to FIG. 13. At a first step, 312 outputs of standard clinical analyte concentration tests are received, and other sensor information and/or user input information are received. In a second step 314 training datasets, each training dataset comprising bodily parameter data, which are diffuse reflectance spectra, are received. A third step 316 is a pre-processing step in which each training dataset is validated to determine whether the training dataset has been acquired from a human user, or to determine whether the training dataset includes outlying data. In a fourth step 318, the training datasets are mapped to the outputs of the standard clinical analyte concentration tests. In a fifth step 320, a health score is calculated for the training datasets using the outputs of the standard clinical analyte concentration tests. In a sixth step 322, the offline model is developed using a PCA model and a regression model. In a seventh step 324, trends between the health score and the other sensor information and/or user input information are investigated. The seventh step 324 is discussed in more detail below.

Thus, training data is collected which comprises a plurality of training datasets, each training dataset being a dataset which comprises bodily parameter information. Each training dataset is associated with a clinical label, where each clinical label is associated with a respective output of the standard clinical analyte concentration test outputs. The clinical data which is used to derive the standard clinical analyte concentration test outputs are acquired at a similar time to the acquisition of the corresponding training dataset, for example within the same 5 minute interval, 15 minute interval or 1 hour interval.

In this way, the offline model effectively maps the data to outputs of standard clinical analyte concentration tests.

Returning to the computer-implemented method 1 shown in FIG. 1, the fourth step 5 of the computer-implemented method is integrating the data with other sensor information, user-input information or learnt metrics.

At this step, 5 the computer-implemented method comprises acquiring other sensor information and/or user input information.

The other sensor information may include, for example, body temperature information obtained from a temperature sensor, heart rate information obtained from a heart rate sensor, blood oxygen saturation information obtained from a blood oxygen saturation sensor, respiratory rate information obtained from a respiratory rate sensor, hydration information obtained from a hydration sensor, accelerometer and motion information obtained from an accelerometer or a motion sensor, heart rate variability information obtained from a heart rate sensor, alcohol concentration, sleep/wake information obtained from a sleep sensor, and blood pressure information obtained from a blood pressure sensor.

The user input information may be acquired from a user input into the wearable device or from a user input into an external device such as a mobile phone. The user input information may include, for example, weight information, height information, activity information, diet information, fluid intake information, sodium intake information, illness information, intoxication information, blood pressure information, sleep duration information, sleep quality information, gender information, age information and heart rate information.

Returning to the computer implemented method 1 shown in FIG. 1, the fifth step 6 of the computer-implemented method is determining a user prompt. The user prompt which is determined may include a recommendation, time-correlated health score information and/or time correlated analyte concentration value information.

To determine a recommendation, the computer-implemented method may comprise storing a recommendation data table which associates recommendations with stored parameters. In this case, the computer-implemented method comprises comparing a derived parameter value with the stored parameter values respectively and, based on this comparison, selecting a recommendation to output to the user.

The parameter values may be health scores, durations of time in which a user's analyte concentration remains within a given sub-range, rates at which a user's analyte concentration changes, analyte concentration values, or peak analyte concentration values of a plurality of analyte concentration values.

For example, the derived parameter value may indicate that the user's blood glucose level is too high. The recommendation associated with this parameter value may be for the user to conduct activity.

The recommendation data table may further associate recommendations with stored other sensor information and/or user input information. The computer-implemented method may comprise comparing acquired other sensor information and/or acquired user input information with stored other sensor information and/or stored user input information respectively. Selecting the recommendation to output to the user may be further based upon this comparison.

To determine a recommendation, the computer-implemented method may comprise applying a user-recommendation machine learning model to the health score.

The user-recommendation machine learning model is trained using training data comprising a plurality of health scores and a respective plurality of context labels. Each of the context labels may comprise user input information and/or other sensor information corresponding to the respective health score. As shown in FIG. 19, the user input information and/or other sensor information may be collected at step 1 212 of generating the offline model. Applying the context labels to the health score may be carried out at step 7 324 of generating the offline model.

To determine time-correlated health score information, the computer-implemented method may comprise a step of storing the health score and storing the time at which the health score is derived. The computer-implemented method thus comprises obtaining time-correlated health score information from previously derived stored health scores and their corresponding stored times.

To determine time-correlated analyte concentration value information, the computer-implemented method may comprise a step of storing the derived analyte concentration value and storing the time at which the analyte concentration value is derived. The computer-implemented method thus comprises obtaining, from previously derived stored analyte concentration values and their corresponding stored times, time-correlated analyte concentration value information.

The computer-implemented method may further comprise a step of storing the other sensor information and/or user input information. The computer-implemented method may thus comprise obtaining time-correlated other sensor information and/or time correlated user input information from previously derived stored other sensor information and/or user input information and their corresponding stored times.

Returning to the method 1 shown in FIG. 1 the sixth step 7 of the computer-implemented method shown in FIG. 1 is delivery of an output to the user.

The output may be displayed to the user on the wearable device, or on an external device such as a mobile phone.

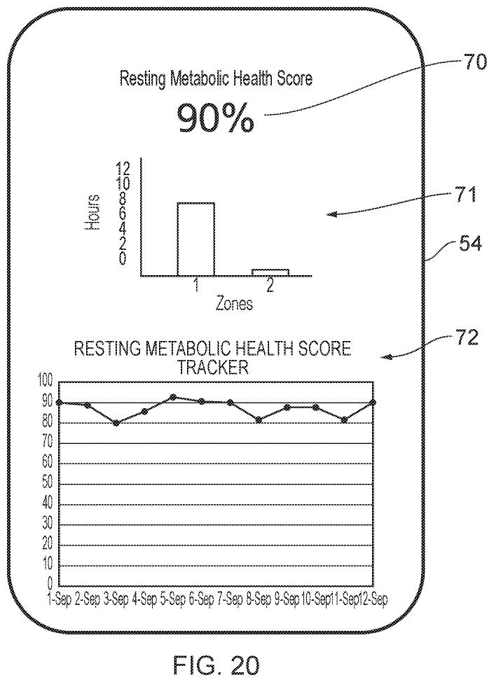

FIG. 20 shows an example of an output being displayed to a user on a mobile device which is a mobile phone 54. The output includes a derived health score 70 which is 90%.

The output further includes a bar chart 71 showing the duration that the user's analyte concentration values spent in each zone over a period of approximately 10 hours.

The output also includes time-correlated health score information 72, which shows the variation of a user's health score over a period of 12 days.

FIG. 8 shows a flow chart 40 for a method which includes embodiments of the computer-implemented method of the present invention. At a first step 41, the user wears a wearable device such that it is in contact with their skin. At a second step 42, data is acquired from sensors of the wearable device. At a third step 43, other sensor information, user input data, and learnt parameters are acquired. At a fourth step 44, an output is provided to the user. At a fifth step 45, the user makes a choice based on the output. At a sixth step 46, data is re-acquired from the sensors. The third step 43 to the sixth step 46 are carried out in a loop. At a seventh step 47, which follows the fifth step, time-correlated information and learnt parameters are acquired.

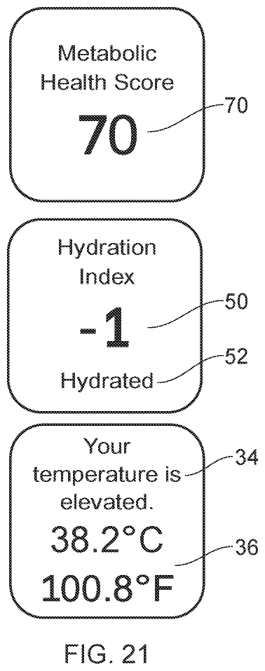

According to an embodiment of the invention, the computer-implemented method may comprise deriving a plurality of physiological ranks. For example, the computer-implemented method may comprise deriving a temperature, a hydration rank and a health score. FIG. 21 shows an example of an output which may be output to a user. The output includes the user's health score 70 hydration index 50, hydration status 52, temperature 36, and a notification 34 that the user's temperature is elevated.

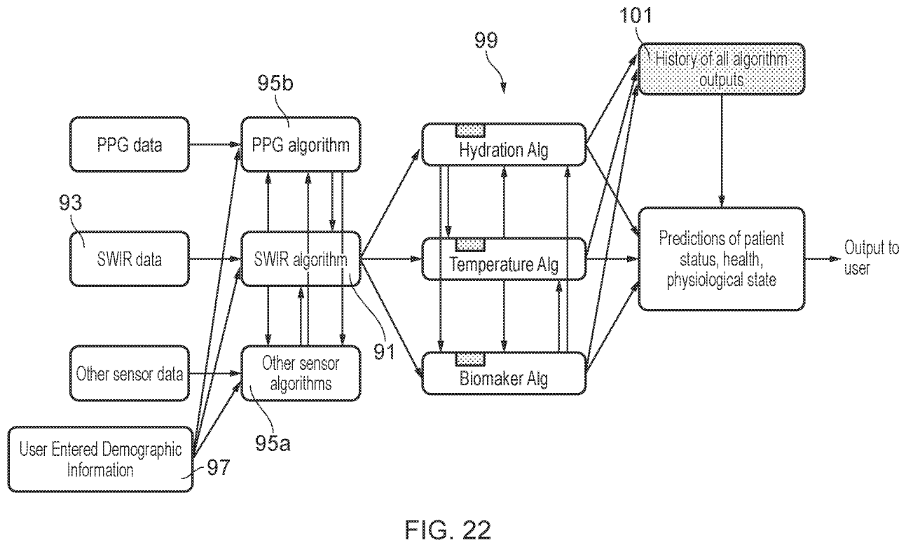

As shown in FIG. 22, the model which derives the physiological rank may include a short-wavelength infrared (SWIR) algorithm 91. The SWIR algorithm may take SWIR data 93 which includes bodily parameter data as an input and may output physiological information. As shown in FIG. 1, the SWIR algorithm may further take as inputs, the outputs of other algorithms 95a/b. In this way, the SWIR algorithm may be calibrated. The SWIR algorithm and the other algorithms may further take as an input user entered demographic information 97. In this way, the SWIR algorithm and the other algorithms may be calibrated.

As further shown in FIG. 22, each model may include a step 99 of deriving the physiological rank and/or recommendations or notifications etc. The model may further include the step of storing time-correlated physiological rank information 101.

As further shown in FIG. 22, derived physiological ranks and/or physiological information from each of the models may be combined. For example, the computer-implemented method may comprise outputting a recommendation based upon both the derived temperature and the derived hydration index. Further, the computer-implemented method may comprise deriving an overall rank based on the combination of the derived temperature and the derived hydration index.

Although exemplary embodiments of a computer-implemented method have been specifically described and illustrated herein, many modifications and variations will be apparent to those skilled in the art. Accordingly, it is to be understood that a computer-implemented method constructed according to principles of this invention may be embodied other than as specifically described herein. The invention is also defined in the following claims, and equivalents thereof.

What is claimed is:

1. A computer-implemented method for deriving a physiological rank indicative of a physiological status of a user, the computer-implemented method comprising:
acquiring, from a sensor on a wearable device worn by a user, data including bodily parameter data related to the user; and,
applying a model to the bodily parameter data to obtain physiological information related to the user; and,
deriving, from the physiological information, a physiological rank indicative of a physiological status of the user wearing the device, wherein the physiological rank is a given value on a physiological rank scale.

2. The computer-implemented method of claim 1 further comprising:
acquiring other sensor information in addition to the physiological information; and/or,
acquiring user input information.

3. The computer-implemented method of claim 2 further comprising:
storing a notification data table, the notification data table associating notifications with stored physiological ranks; and,
comparing the derived physiological rank with the stored physiological ranks and, based on this comparison,
selecting a notification to output to the user; and,
outputting the selected notification to the user.

4. The computer-implemented method of claim 3, wherein the notification data table further associates notification with stored other sensor information and/or stored user input information, and wherein the method further comprises:
comparing acquired other sensor information and/or acquired user input information with the stored other sensor information and/or the stored user input information,
wherein the selecting the notification to output to the user is further based upon this comparison.

5. The computer-implemented method of claim 1 wherein the physiological information is temperature information, and wherein the physiological rank is a temperature rank of the user.

6. The computer-implemented method of claim 5 wherein the temperature rank is an output of a temperature value or a temperature status.

7. The computer-implemented method of claim 5 wherein the bodily parameter data is a body tissue absorption spectrum.

8. The computer-implemented method of claim 5 further comprising:
acquiring other sensor information in addition to the physiological information; and/or,
acquiring user input information,
wherein the other sensor information includes one or more of hydration information obtained from a hydration sensor, heart rate information obtained from a heart rate sensor, blood pressure information obtained from a blood pressure sensor, activity information obtained from an accelerometer, and climate information obtained from a climate sensor.

9. The computer-implemented method of claim 5, further comprising:
acquiring other sensor information in addition to the physiological information; and/or,
acquiring user input information,
wherein the user input information includes one or more of cervical mucous status and date of menstruation.

10. A computer-implemented method for deriving a physiological rank indicative of a physiological status of a user, the computer-implemented method comprising:
applying a model to bodily parameter data acquired from a user to obtain physiological information related to the user; and,
deriving, from the physiological information, a physiological rank indicative of a physiological status of the user wearing the device, wherein the physiological rank is a value on a physiological rank scale.

11. The computer-implemented method of claim 5, further comprising a step of determining a hydration status of a user, the computer-implemented method further comprising:
acquiring, from a sensor on a wearable device worn by a user, data including additional bodily parameter data related to the user; and,
applying a model to the bodily parameter data to obtain hydration information related to the user; wherein,
the model deriving, from the hydration information, a hydration rank indicative of a hydration status of the user, wherein the hydration rank is a given grade on a hydration rank scale.

12. The computer-implemented method of claim 11 wherein each hydration rank on the hydration ranks scale maps onto a respective output of a standard clinical point of care test.

13. The computer-implemented method of claim 12 wherein the standard clinical point of care test is a test of urine osmolality, urine specific gravity, fluid gain, fluid loss, increases or decreases in body weight or mass representing fluid gain or fluid loss, respectively, or serum osmolality.

14. The computer-implemented method of claim 11 wherein the hydration rank is a hydration index, and wherein the hydration index is a given value on a hydration index scale.

15. The computer-implemented method of claim 14 wherein each hydration index on the hydration index scale maps onto a respective output of a standard clinical point of care test.

16. The computer-implemented method of claim 14 wherein the hydration index scale is sub-divided into a plurality of sub-ranges of hydration index values, each of the plurality of sub-ranges corresponding to a different clinical hydration status of the user, and wherein the method further comprises:
determining which sub-range of the plurality of sub-ranges the hydration index value falls within.

17. The computer-implemented method of claim 11 wherein the hydration rank is a clinical hydration status of the user.

18. The computer-implemented method of claim 17 further comprising:
outputting the clinical hydration status of the user, or the hydration index.

19. The computer-implemented method of claim 11 wherein the bodily parameter data includes a water absorption spectrum.

20. The computer-implemented method of claim 11 wherein the model includes a regression model.

21. The computer-implemented method of claim 5, further comprising a determination of how well a user's body is regulating an analyte, the computer-implemented method further comprising:
acquiring, from a sensor on a wearable device worn by the user, data including bodily parameter data related to the user; and,
applying a model to the bodily parameter data to obtain analyte concentration information related to the user, wherein,
the model derives, from the analyte concentration information, a health score indicative of how well the user's body is regulating the analyte, wherein the health score is a given grade on a health score scale.

22. A wearable device comprising a processor, the processor configured to carry out the computer-implemented method of claim 1.
